(19) United States
(12) Patent Application Publication
(10) Pub. No.: US 2012/0151612 A1
(43) Pub. Date: Jun. 14, 2012

(54) NON-SURGICAL APPROACH TO PREVENT AND CORRECT CRANIOFACIAL MALFORMATIONS DURING DEVELOPMENT

(75) Inventors: Yang CHAI, Pasadena, CA (US); Junichi IWATA, South Pasadena, CA (US)

(73) Assignee: UNIVERSITY OF SOUTHERN CALIFORNIA, Los Angeles, CA (US)

(21) Appl. No.: 13/296,165

(22) Filed: Nov. 14, 2011

Related U.S. Application Data
(60) Provisional application No. 61/412,947, filed on Nov. 12, 2010.
(60) Provisional application No. 61/413,354, filed on Nov. 12, 2010.
(60) Provisional application No. 61/413,366, filed on Nov. 12, 2010.
(60) Provisional application No. 61/557,837, filed on Nov. 9, 2011.

(51) Int. Cl. A61K 39/395; A61K 31/4439; A61K 31/4178; C12Q 1/68; A01K 67/027; A61P 43/00; G01N 33/566; C40B 30/04; A61K 31/4184

(52) U.S. Cl. 800/9; 506/9; 514/341; 514/381; 514/394; 424/172.1; 435/7.1; 435/6.12

(57) ABSTRACT

The present invention discloses a novel TGF-β signaling mechanism implicated in craniofacial malformation as well as methods and compositions for treating craniofacial malformation utilizing knowledge of the mechanism. Methods of the invention generally comprises administering an effective amount of a TGF-β inhibitor to a subject in need of the treatment. Also disclosed are methods for treating craniofacial malformation by administering Tgf-β, Tgf-βRIII, p38 MAPK inhibitor or neutralizing antibodies to a subject. Also disclosed is a diagnostic method for diagnosing patients at risk of developing craniofacial malformation by determining the level of Tgf-β2 and ectopic p38 MAPK activation. Compounds useful for treating craniofacial malformation may also be discovered by using animal models of the present invention.

### CROSS-REFERENCE TO RELATED APPLICATIONS

This application claims benefit under 35 USC §119(e) to Provisional Applications Nos. 61,412,947, 61/413,354, and 61/413,366, all of which were filed on Nov. 12, 2010. This application also claims benefit to Provisional Application No. 61/557,837, filed on Nov. 9, 2011. The entire contents of the above priority applications are hereby incorporated herein by reference.

### STATEMENT REGARDING FEDERALLY SPONSORED RESEARCH AND DEVELOPMENT

This invention was made with government support under Contract No. R01 DE012711 and DE020065 awarded by the National Institute of Health and the National Institute of Dental and Craniofacial Research. The government has certain rights in the invention.

### SEQUENCE LISTING

This application contains sequence listing.

### FIELD OF THE INVENTION

The present invention relates in general to craniofacial malformations. More specifically, the invention provides methods and compositions for prevention and non-surgical correction of craniofacial malformation. The invention also provides genetically engineered mice as animal research model for studying craniofacial malformation, and methods for discovering new drug leads for non-surgical treatment of craniofacial malformation.

### BACKGROUND OF THE INVENTION

Craniofacial deformities, such as cleft palates or lips, are among the most common congenital birth defects in humans, affecting approximately 1 in every 700 live births worldwide. A cleft palate/lip may impact an individual's self-esteem, social skills, and behavior, particularly among girls. There is a large amount of research dedicated to the psychological development of individuals with cleft palate. Moreover, cleft may cause problems with feeding problems, ear disease, or speech impairment.

Diagnoses of craniofacial malformation traditionally could only be done at birth. At this point, a team of healthcare professionals will be responsible for managing and treating the condition throughout the patient's life. Expensive and painful surgical intervention are usually prescribed as the patient approaches adolescence. In some cases of a sever bi-lateral complete cleft, the premaxillary segment will be protruded far outside the mouth. Nasoalveolar molding may be required prior to surgery. Although recent advances in technology has enabled in utero imaging, diagnosing craniofacial malformation still required awaiting the fetus to develop symptoms.

Therefore, it would be highly desirable to be able to diagnose craniofacial malformation during early stages of development, even before the malformation takes shape and apply non-surgical interventions to correct or prevent the malformation.

### SUMMARY OF THE INVENTION

In view of the above, it is one object of the present invention to provide methods for diagnosing craniofacial malformation in a developing subject.

It is also an object of the present invention to provide methods and compositions for treating or preventing craniofacial malformation in a subject in need of said treatment.

It is a further object of the present invention to provide tools and methods for identifying compounds that are useful as potential drugs for treating or preventing craniofacial malformation.

Methods, tools and compositions that satisfy the objects of the present invention are based, in part, on the unexpected discovery and elucidation of a heretofore undiscovered mechanism of TGF-β signaling and its role in cell proliferation defects of the palatal mesenchyme that result in cleft palate and other craniofacial deformities.

Accordingly, one aspect of the present invention is directed to the discovery that craniofacial malformation may be due to cell signaling network defects as a result of genetic mutations. In particular, the present invention has discovered that mutation in transforming growth factor beta (TGF-β) signaling results in both humans and mice. Significantly, it was also discovered that established medications such as Telmisartan are able to rescue cleft palate defects in mice by inhibiting TGF-β signaling.

Therefore, in one aspect, the present invention provides a method for treating a subject at risk of developing craniofacial deformities or is in the process of developing craniofacial deformities by administering to the subject an effective amount of a TGF-β inhibitor or modulator.

In one preferred embodiment, the TGF-β inhibitor or modulator is Telmisartan. In one preferred embodiment, the craniofacial deformity is cleft palate. In another preferred embodiment, the craniofacial deformity is Marfan, Loeys-Dietz syndrome, DiGeorge syndrome, Smith-Lemli-Opitz syndrome, lathosterolosis, desmosterolosis, X-linked dominant chondrodysplasia punctata type 2, congenital hemidysplasia with ichthyosiform erythroderma and limb defects.

In general, subject in need of the treatment are preferably fetuses in early stages of development. In a human subject, craniofacial structures begins to develop within the first trimester, hence, treatment methods of the present invention is preferably administered during the first trimester. Because the subject is still a fetus in at this stage, administration of the inhibitor or modulator can be direct or indirect. Direct administration can be achieved by any direct in utero method of delivering a pharmaceutical to the fetus. In some instances, the fetus may be incubated and grown outside of the mother's uterus. In other cases, the fetus is being carried by the mother or a host. In such cases, administration may be done via the mother or host. For example, Telmisartan may be administered to a mother who is carrying a fetus that has been diagnosed as being at risk of developing cleft palate.

In another aspect, the present invention is directed to the discovery that elevated Tgf-β activity and a resulting ectopic p38 MAPK activation is responsible for adversely affecting cell proliferation in the CNCC-derived palatal mesenchyme. Accordingly, one aspect of the present invention is directed to the use of elevated Tgf-β2 and/or ectopic p38 MAPK activation as diagnostic biomarkers for patients, including fetuses, that have or at risk for cell proliferation defects that result in cleft palate and other craniofacial deformities. Methods for diagnosing patients subject to or at risk for cleft palate or other craniofacial deformities in accordance with this aspect of the present invention generally comprises the steps of determining a level of Tgf-β2 and/or ectopic p38 MAPK activation in a patient and comparing the level to a control level, wherein when the level of Tgf-β2 and/or ectopic p38 MAPK activation in the patient is higher than a control level, the patient, or the patient's fetus, is at risk for cleft palate or other craniofacial deformities. Those skilled in the art will understand that suitable controls may be established by measuring the Tgf-β2 level and/or ectopic p38 MAPK activity in healthy individuals.

The term “biomarker” refers a biological molecule, e.g., a nucleic acid, peptide, hormone, etc., whose presence or concentration can be detected and correlated with a known condition, such as a disease state. Another aspect of the present invention is directed to the use of elevated Tgf-β2 and/or ectopic p38 MAPK activation as diagnostic biomarkers for patients, including fetuses, that have or at risk for cell proliferation defects that result in cleft palate and other craniofacial deformities. Thus, one aspect of the present invention are methods for diagnosing patients subject to or at risk for cleft palate or other craniofacial deformities comprising testing a level of Tgf-β2 and/or ectopic p38 MAPK activation in a patient and comparing the level to a control level, wherein when the level of Tgf-β2 and/or ectopic p38 MAPK activation in the patient is elevated, or higher, than a control level, the patient, or the patient's fetus, is at risk for cleft palate or other craniofacial deformities. The level of Tgf-β should be about twice as high as the control group. The level of Tgf-β may be measured quantitatively by known methods, for instance, using Western analysis.

In the absence of Tgfbr2, elevated Tgf-β2 forms a complex with Tgf-βRIII/Tgf-βRI/β-spectrin and induces p38 MAPK signaling cascade in the palatal mesenchyme (FIG. 12e). The activation of this novel Tgf-β type I/III receptor-mediated p38 MAPK signaling pathway results in a subsequent cell proliferation defect in the palatal mesenchyme. The elevated Smad-independent p38 MAPK activation mediated through Tgf-βRI/RIII is responsible for the CNCC proliferation defect and failure of palatal fusion. Given the early developmental onset of structural birth defects, modulation of (1) TGF-β signaling at the ligand or receptor level and/or (2) inhibition of the p38 MAPK signaling pathway provides opportunities for treating individuals with altered TGF-β signaling. Therefore, another aspect of the invention is the discovery that cell proliferation defects in the palatal mesenchyme, such as cleft palate, can be corrected, or rescued, via reduction of Tgf-β2 levels.

Accordingly, another aspect of the present invention is directed to compositions and treatment methods for the prevention and rescue cleft palate or other craniofacial defects in patients who have or are at risk for congenital craniofacial malformations by modulating altered Tgf-β2 signaling and/or modulating elevated ectopic p38 MAPK activation. Methods in accordance with this aspect of the invention may seek to correct, or rescue, subjects from developing craniofacial malformation by treatment with a neutralizing antibody for Tgf-βRIII or Tgf-β2. Alternatively, cell proliferation defects can be corrected, or rescued using small molecule p38 MAPK inhibitors. The rescue, or normalization, of cell proliferation can lead to proper formation of the craniofacial structure, and the elimination, or reduction, of the craniofacial defects including cleft palate.

Thus, in this aspect of the invention, there is provided treatment methods which comprise administering to a patient in need thereof an effective amount of a neutralizing antibody for Tgf-βRIII or Tgf-β2 or a combination thereof. Another aspect of the present invention is a treatment method which comprises administering to a patient in need thereof an effective amount of a Tgf-β2 or p38 MAPK inhibitor or modulator. The p38 MAPK inhibitor may be the p38 MAPK inhibitor SB203580

In a preferred embodiment, methods in accordance with this aspect of the invention comprises administering to a patient in need thereof an effective amount of a Tgf-β2 or p38 MAPK inhibitor or modulator. The p38 MAPK inhibitor may be the p38 MAPK inhibitor SB203580. The IUPAC name of SB203580 is 4-[4-(4-fluorophenyl)-2-(4-methylsulfinylphenyl)-1H-imidazol-5-yl]pyridine. It has the following structure:

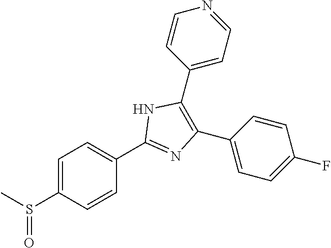

An effective amount of a Tgf-β2 or p38 MAPK inhibitor or modulator is preferably an amount sufficient to reduce the level of Tgf-β2 or p38 MAPK activation to a level where cell proliferation occurs at rate sufficient to minimize or eliminate any cell proliferation defect, thereby resulting in normal craniofacial development.

It should be noted that it is preferred, but not necessary, that all craniofacial defects be prevented. Rather, successful treatment of the present invention occurs where there the extent of the cell proliferation defect is diminished and/or the risk of craniofacial defect is lessened. In this connection, “Treating” or “Treatment” refers to both therapeutic treatment and prophylactic or preventative measures, wherein the object is to prevent or lessen the targeted pathologic condition or disorder. Those in need of treatment in connection with this invention include those already with the disorder as well as those prone to have the disorder or those in whom the disorder is to be prevented.

In another preferred embodiment, methods in accordance with this aspect of the present invention is directed to treatment methods which comprise administering to a patient in need thereof an effective amount of a neutralizing antibody for Tgf-βRIII or Tgf-β2 or a combination thereof. The antibody, or antibody fragment, may be any monoclonal or polyclonal antibody that specifically recognizes Tgf-βRIII or Tgf-β2. The monoclonal antibodies, or fragments thereof, may include chimeric or humanized antibodies that specifically bind to the Tgf-βRIII or Tgf-β2. In other embodiments, the monoclonal antibodies, or fragments thereof, are human antibodies that specifically bind to Tgf-βRIII or Tgf-β2. An effective amount of an antibody according to this invention is preferably an amount sufficient to produce cell proliferation in an amount sufficient to minimize or eliminate any cell proliferation defect, thereby resulting in limited craniofacial defect. It should be noted that it is preferred, but not necessary, that all craniofacial defects be prevented. Successful treatment of the present invention occurs where there the extent of the cell proliferation defect Is diminished and/or the risk of craniofacial defect is lessened.

The above discoveries were made with the help of animal models which mimic the pathology of craniofacial malformation due to genetic mutation. Accordingly, these engineered animals also serve as excellent research tools for identifying novel compounds that can act to rescue or prevent craniofacial malformation.

While the above described embodiments outlines the general aspects of the present invention, it will be understood by those skilled in the art that various modifications are possible. Other aspects and advantages of the present invention will become apparent from the following detailed description, the accompanying figures and the appended claims.

### BRIEF DESCRIPTION OF THE DRAWINGS

FIG. 1. Oral-nasal patterning of the palate. A-B, Toluidine blue staining of semi-thin sections of newborn head showing regional morphology of the palatal epithelium of wild type mice: a coronal section of the palate (A) and an enlarged palatal shelf (B). Box in B is enlarged in insert, and shows pseudostratified, ciliated, columnar epithelia that cover the nasal side of the palatal shelf. Broken lines outline the palatal bone. C-F, Expression of nasal side markers in coronal sections of the palate at E13.5 assayed by ISH for Dlx5 (C) and Fgf7 (D) and immunostaining of phospho-Smad1/5/8 (E, F). F is enlarged from the box in E. G-K, Expression of oral side markers in coronal sections of the palate at E13.5 assayed by ISH of Shh (G), Gli1 (H, J), and Fgf10 (I, K). J and K are enlargements of boxes in H and I, respectively. ISH: in situ hybridization. Scale bar=200 μm.

FIG. 2. Excessive growth of the oral side of the palate in Dlx5−/− mice. A-C, Scanning electron microscopy (SEM) of newborn wild type and Dlx5−/− mice heads (oral view). Some (11%) Dlx5−/− mice have a groove in the hard palate (C). Vertical line: the fusion midline of palatal shelves (A). Black arrow: posterior edge of soft palate (A, B). White arrow: rugae (A, B). Arrowhead: folding of the palate shelf (C). Broken line (C): the plane of the section shown in D. D, H & E staining of coronal section of the Dlx5−/− newborn head in C. E-F, H & E staining of sagittal sections of newborn head. White arrow: rugae (E, F). Broken lines (below figure F): trace of the oral epithelium. G-O, Whole-mount and section ISH of Shh in oral epithelium at E12.5 (G, H), E13.5 (I-L) and E14.5 (M-O) in wild type and Dlx5−/− mice. Arrows: the expression domain of Shh in the palatal epithelium (I-L). Inserts are the enlarged boxed areas in I and J. Whole-mount 30 ISH of Shh in palatal epithelium at E14.5 also includes Msx1−/− mice (M-O). Open arrows: expanded Shh expression in Dlx5−/− mice (N) and diminished Shh expression in Msx1−/− mice (O). Scale bar 1 mm (A-C); 200 μm (E-L). H & E: Hematoxylin and eosin; ISH: in situ hybridization.

FIG. 3. Altered oral-nasal patterning in Dlx5−/− palate. A-C, ISH of Gli1 in coronal sections of E13.5 wild type (A, arrowhead indicates the anterior-posterior groove between the palatal process and the body of the maxilla), Dlx5−/− (B), and Msx1−/− palate (C). D-F, Immunostaining of phospho-Smad1/5/8 in coronal sections of E13.5 wild type (D), Dlx5−/− (E), and Msx1−/− palate (F). G-I, ISH of Fgf7 in coronal sections of E13.5 wild type (G), Dlx5−/− (H), and Msx1−/− palate (I). J, Real-time PCR quantification of Fgf7 mRNA abundance in wild type, Dlx5−/− and Msx1−/− palatal tissue. Arrows and arrowheads: the expression domains of Fgf7. Dashed lines divide palatal shelves into nasal and oral domains. ISH: in situ hybridization. Scale bar=200 μm.

FIG. 4. Shh signaling affects palatal mesenchymal proliferation. A, B Whole-mount and sagittal section ISH of Shh in palatal epithelium at E13.5. Lines in A indicate the section plane in B. *: the expression of Shh in palatal epithelium (B). C-D, BrdU staining of sagittal sections of E13.5 palate. Broken line: the outline of the palate and the foci of proliferating palatal mesenchymal cells (D). *: ruga where Shh is expressed (D). E-G, BrdU staining of the anterior part of the secondary palates of E14.0 wild type (E), Msx1−/− (F), and Msx1−/−; Dlx5−/− (G) embryos. Dashed lines define the area for BrdU index analyses (below dashed lines). Arrows point at BrdU positive cells. “*” indicates reduced cell proliferation (F). “**” indicates restored cell proliferation (G). H, BrdU labeling index in wild type, Msx1−/− and Msx1−/−; Dlx5−/− palatal mesenchyme. Error bars indicate 95% confidence intervals. I-L, Gross appearance of palatal shelves extension at E14.5 in wild type (I), Msx1−/− (J), Dlx5−/− (K) and Msx1−/−; Dlx5−/− (L) embryos. Scale bar=200 μm.

FIG. 5. Msx1 and Dlx5 expression in the developing palatal shelf at E13.5 and the rescue of cleft palate in Msx1−/−; Dlx5−/− mice. A-D, Oral view of the palate at E17.5. Arrow: the midline of palate fusion or the palatal shelf (B). E-H, H & E staining of coronal sections of newborn heads. Arrowhead: palatal shelf (F) or the palate. I-L, Alcian Blue and Alizarin Red staining of palates. Black broken lines: outline of maxillary palatine process. Green broken line: horizontal part of the palatine bone. Blue arrow: anterior part of the secondary palate. mx: maxilla, pl: palatine, ppmx: palatal process of maxilla, pppl: palatal process of palatine, PS: palate shelf, T: tooth, vm: vomer. M-P, Oral view of whole mount ISH of Msx1 (M, wild type; N, Dlx5−/−) and Dlx5 (O, Msx1−/−; P, wild type) in the palate at E13.5. Arrow: Msx1 or Dlx5 expression in the palatal shelf. Q-T, ISH of Msx1 (Q, wild type; R, Dlx5−/−) and Dlx5 (S, Msx1−/−; T, wild type) in coronal sections of E13.5 palate. *: Msx1 expression. Black arrows: Dlx5 expression domain in the nasal side of the palatal shelf. H & E: Hematoxylin and eosin; ISH: in situ hybridization. Scale bar=200 μm.

FIG. 6. Shh signaling is responsible for cell proliferation in the palate mesenchyme. AB: BrdU immunostaining of E13.5 wild type palatal shelves treated with BSA or Shh beads. Insert: enlarged open box. Arrow: proliferating cells. C, BrdU labeling index in BSA and Shh beads treated palatal mesenchyme. Error bars indicate 95% confidence interval. D-E, TUNEL assay of E13.5 wild type palatal shelves treated with BSA or Shh beads. Arrowhead: apoptotic signal. F-K: BrdU immunostaining of wild type (F, I), Dlx5−/− (G, J) and Msx1−/−; Dlx5−/− (H, K) palatal shelves treated with BSA (bovine serum albumin) or anti-Shh antibody (Anti-Shh). Arrows: proliferating mesenchymal cells. Scale bar: 100 μm. L, BrdU index of panels F-K. M-P, BrdU immunostaining (arrows) of wild type (M, O) and Msx1−/− palatal shelves treated with control mouse IgG1 or Anti-Fgf7 neutralizing antibody. Q, BrdU index of panels M-P. Scale bar=200 μm.

FIG. 7. Regulation of Shh signaling in the developing palate. A-D, ISH of coronal sections of E13.5 mouse embryos, showing Shh expression in the palatal epithelium. E, Whole-mount ISH of Shh in wild type palatal explants treated with BSA beads (blue) and Fgf7 beads (white). Arrow: the expression of Shh in the tooth bud served as control. FG, ISH of Shh in coronal sections of palatal explants treated with BSA and Fgf7 beads. H-K, Oral view of whole mount ISH of Shh in palatal explants treated with IgG1 (H, wild type; J, Msx1−/−) or Anti-Fgf7 neutralizing antibody (I, wild type; K, Msx1−/−). L, Oral view of whole mount ISH analysis of Fgf7 expression (arrows) in palatal explants treated with BSA beads and Shh beads. M-N, ISH analysis of Fgf7 expression in coronal sections of palatal explants treated with BSA and Shh beads. O, Schematic drawing depicting the antagonistic regulation of Msx1 and Dlx5 on Shh expression to control mesenchymal cell proliferation during palatogenesis. T: tongue, P: palate. ISH: in situ hybridization. Scale bar=200 μm.

FIG. 8. Soft palate patterning defects in Dlx5−/− newborn mice. A-B, Scanning electron microscopy (SEM) of newborn wild type and Dlx5−/− mice heads (oral view). Arrow (in A): the attachment of the posterior palate to the posterior pharyngeal wall. Arrow (in B): the uvula-like structure in Dlx5−/−. Blue box: sagittal section of the posterior soft palate shown in C and D. C-D Sagittal section of newborn head. Arrow: posterior border of the soft palate. Broken line shows normal soft palate in C and shortening of soft palate in Dlx5−/− mice (D). E-F, Excessive air retention in the stomach of Dlx5−/− newborn mice. Wt: wild type. Scale bar 1 mm.

FIG. 9 Excessive growth of the oral-side palatal epithelium in Dlx5−/− mice. H & E staining of coronal section of wild type (A, C) and Dlx5−/− (B, D) mice at newborn stage. Boxed areas are enlarged in C and D. Arrows: oral-side palatal epithelium (thickened in D versus C); arrowheads: nasal-side palatal epithelium (comparable in C and D). H & E: Hematoxylin and eosin.

FIG. 10 Loss of Fgf7 expression in nasal side of palatal mesenchyme in Dlx5−/− and Msx1−/−; Dlx5−/− mice. ISH analysis of Fgf7 expression (dark blue) on coronal sections of E13.5 palatal shelves of wild type (A), Dlx5−/− (B), and Msx1−/−; Dlx5−/− (C) embryos. Arrowhead: anterior-posterior groove between the palatal process and the body of the maxilla. ISH: in situ hybridization. Scale bar=200 μm.

FIG. 11 Altered Tgf-β signaling pathway in the palate of Tgfbr2^{fl/fl}; Wnt1-Cre mice. (a) Microarray analyses using palates of Tgfbr2^{fl/fl} and Tgfbr2^{fl/fl}; Wnt1-Cre mice at E14.5. Differential expression of Tgfb and Tgfbr family members is listed. ^{a}Ratio of the geometrix means of Tgfbr2^{fl/fl}; Wnt1-Cre and Tgfbr2^{fl/fl} control mice. ^{b}DR calculated-based on the global microarray analysis (see Methods). *Transcripts showing >1.5-fold change with <5% FDR. (b) Quantitative RT-PCR analyses of indicated genes in the palates of Tgfbr2^{fl/fl} and Tgfbr2^{fl/fl}; Wnt1-Cre mice at E 13.5 and E14.5. *, p<0.05 as indicated by a two-tailed Student's t-test. (c) Immunoblotting (IB) analysis of indicated molecules in primary MEPM cells of Tgfbr2^{fl/fl} and Tgfbr2^{fl/fl}; Wnt1-Cre mice, (d) IB analyses of p38 MAPK and 14-3-3ζ/δ in primary MEPM cells of Tgfbr2^{fl/fl} and Tgfbr2^{fl/fl}; Wnt1-Cre mice treated with (+) or without (−) p38 MAPK inhibitor SB203580. Uncropped images of blots are shown in Supplementary information, FIG. 22.

FIG. 12 Altered ligand/receptor assembly and up-regulated phosphorylation and ubiquitination of Tak1 in the absence of Tgfbr2. (a) IB analysis of indicated molecules in primary MEPM cells from Tgfbr2^{fl/fl} and Tgfbr2^{fl/fl}; Wnt1-Cre mice cultured with Tgf-β2 (10 ng/ml) for indicated time. (b) IB analysis of immunoprecipitation (IP) products derived from Tgfbr2^{fl/fl} (lane 1) and Tgfbr2^{fl/fl}; Wnt1-Cre (lane 2) MEPM cell abstracts (Input) using the indicated antibodies. Uncropped images of blots are shown in Supplementary information, FIG. 22. (c) Cross-linking analysis after treatment with radioactive Tgf-β in primary MEPM cells from Tgfbr2^{fl/fl} and Tgfbr2^{fl/fl}; Wnt1-Cre mice, (d) IB analysis with anti-ubiquitin (Ub) antibody following IP by anti-Tak1 antibody of extracts from MEPM cells (Input) of Tgfbr2^{fl/fl} and Tgfbr2^{fl/fl}; Wnt1-Cre mice. Uncropped images of blots are shown in Supplementary information, FIG. 22. (e) Schematic diagram indicates the mechanism of p38 MAPK activation in Tgfbr2^{fl/fl}; Wnt1-Cre palate. Tgf-β2 is up-regulated and binds to Tgf-βRIII, followed by assembly with Tgf-βRI and β-spectrin. Tak1 is ubiquitinated (U) and phosphorylated (P) following Tgf-β2 binding. Finally, p38 and 14-3-3 proteins are phosphorylated.

FIG. 13 Rescued cleft palate in Tgfbr2^{fl/fl}; Wnt1-Cre mice via reduction of Tgf-β2 dosage, (a) Morphologies of newborn Tgfbr2^{fl/fl} control, Tgfbr2^{fl/fl}; Wnt1-Cre and Tgfbr2^{fl/fl}; Wnt1-Cre; Tgfb2^{+/-} mice. Bottom views show macroscopic appearance of palate at newborn stage. Arrowheads show calvaria defects. Arrow shows cleft palate, and open arrows show normal palates. Palates were scored as normal or cleft. (b) Hematoxylin and eosin staining of sections of Tgfbr2^{fl/fl}, Tgfbr2^{fl/fl}; Wnt1-Cre, and Tgfbr2^{fl/fl}; Wnt1-Cre; Tgfb2^{+/-} mice at E14.0, E14.5, and E16.5. Arrows indicate palate. Most Tgfbr2^{fl/fl}; Wnt1-Cre; Tgfb2^{+/-} mice show a half-day delay in the elevation of palatal shelves, although some Tgfbr2^{fl/fl}; Wnt1-Cre; Tgfb2^{+/-} palates are indistinguishable from wild-type palates at E14.0. Bar, 50 μm. (c) Whole-mount skeletal staining with Alcian blue-Alizarin Red S of Tgfbr2^{fl/fl}, Tgfbr2^{fl/fl}; Wnt1-Cre, and Tgfbr2^{fl/fl}; Wnt1-Cre; Tgfb2^{+/-} newborn mice, (d) IB analysis of E14.5 Tgfbr2^{fl/fl}, Tgfbr2^{fl/fl}; Wnt1-Cre, and Tgfbr2^{fl/fl}; Wnt1-Cre; Tgfb2^{+/-} mice. Uncropped images of blots are shown in Supplementary information, FIG. 12. (e) BrdU staining of Tgfbr2^{fl/fl}, Tgfbr2^{fl/fl}; Wnt1-Cre, and Tgfbr2^{fl/fl}; Wnt1-Cre; Tgfb2^{+/-} mice at E14.0. Bar, 50 μm. (f) The number of BrdU-labeled nuclei in the palate of Tgfbr2^{fl/fl}, Tgfbr2^{fl/fl}; Wnt1-Cre, and Tgfbr2^{fl/fl}; Wnt1-Cre; Tgfb2^{+/-} mice at E14.0. ***, p<0.001.

FIG. 14 Rescued cleft palate in Tgfbr2^{fl/fl}; Wnt1-Cre mice via reduction of Tgf-β receptor type I dosage, (a) Morphologies of newborn control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}; Wnt1-Cre; Alk5^{fl/fl} mice. Bottom views show macroscopic appearance of palate at newborn stage. Arrowheads show calvaria defects. Arrows show cleft palate, and open arrows show normal palates. Palates were scored as normal or cleft. (b) Hematoxylin and eosin staining of sections of control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}, Wnt1-Cre; Alk5^{fl/fl} mice palates at E14.0 and E14.5. Most Tgfbr2^{fl/fl}; Wnt1-Cre; Alk5^{fl/+} mice show a half-day delay in the elevation of palatal shelves, although some Tgfbr2^{fl/fl}; Wnt1-Cre; Alk5^{fl/+} palates are indistinguishable from wild-type littermate palates at E14.0. Arrows show palate. Bar, 50 μm. (c) Whole-mount skeletal staining with Alcian blue-Alizarin Red S of control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}; Wnt1-Cre; Alk5^{fl/fl} newborn mice. Arrows indicate calvaria defects. (d) IB analysis of E14.5 Tgfbr2^{fl/fl}; Wnt1-Cre (lane 1), control (lane 2), Tgfbr^{fl/fl}; Wnt1-Cre; Alk5^{fl/+} (lane 3), Tgfbr2^{fl/fl}; Wnt1-Cre; Alk5^{fl/fl} (lane 4), and Tgfbr2^{fl/+}; Wnt1-Cre; Alk5^{fl/fl} (lane 5) mice. Uncropped images of blots are shown in Supplementary information, FIG. 22. (e) BrdU staining of control, Tgfbr2^{fl/fl}; Wnt1-Cre, and Tgfbr2^{fl/fl}; Wnt1-Cre; Alk5^{fl/+} mice at E14.0. Bar, 50 μm. (f) Quantification of the number of BrdU-labeled nuclei in the palate of control, Tgfbr2^{fl/fl}; Wnt1-Cre, and Tgfbr2^{fl/fl}; Wnt1-Cre; Alk5^{fl/+} mice at E14.0. ***, p<0.001.

FIG. 15 shows the Expression of genes relevant to Tgf-β signaling in the palate of Tgfbr2^{fl/fl}; Wnt1-Cre mice. Relationships of up-regulated genes (>1.5-fold, <5% FDR) relevant to Tgf-β signaling in the cleft palate of Tgfbr2^{fl/fl}; Wnt1-Cre mice relative to normal palates of Tgfbr2^{fl/fl} mice at E14.5, as highlighted through Ingenuity Pathway Analysis (IPA) software analysis.

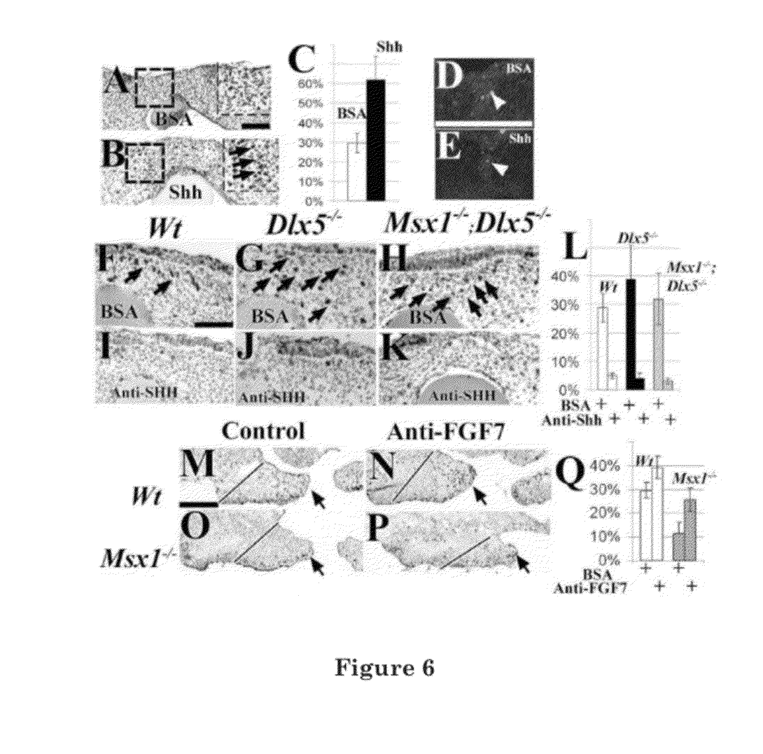

FIG. 6 shows the Up-regulated expression of Tgf-β2 and Tgf-β receptor type III in Tgfbr2^{fl/fl}; Wnt1-Cre palate, (a) Immunoblotting (IB) analysis of E14.5 Tgfbr2^{fl/fl} and Tgfbr2^{fl/fl}; Wnt1-Cre palate. Uncropped images of blots are shown in FIG. 22. (b, c) Immunohistochemical (IHC) staining of Tgf-β2 and DAPI staining in sections of Tgfbr2^{fl/fl} and Tgfbr2^{fl/fl}; Wnt1-Cre mice at E13.5 (b) and E14.0 (c). Tgf-β2 expression is detectable in the palatal mesenchyme of both Tgfbr2^{fl/fl} and Tgfbr2^{fl/fl}; Wnt1-Cre mice, but appears increased in Tgfbr2^{fl/fl}; Wnt1-Cre palate compared to Tgfbr2^{fl/fl} littermate control at E14.0. Arrows show palate. Bar, 50 μm. (d-g) IHC staining of Tgf-βRIII and DAPI staining in sections of Tgfbr2^{fl/fl} and Tgfbr2^{fl/fl}; Wnt1-Cre mice at E13.5 (d), and E14.5 (e, f). Box is shown enlarged on the right (e). Tgf-βRIII expression is detectable in palatal mesenchyme of both Tgfbr2^{fl/fl} and Tgfbr^{fl/fl}; Wnt1-Cre mice, but appears increased in Tgfbr2^{fl/fl}; Wnt1-Cre palate compared to Tgfbr2^{fl/fl} littermate control. Arrows show palate. Open arrows in (f) show no positive signal of Tgf-βRIII in the microcartilage and tongue. Bar, 50 μm.

FIG. 17 identifies the molecules with increased expression in primary MEPM cells from Tgfbr2^{fl/fl}; Wnt1-Cre mice compared to control. (a) LacZ staining of Wnt1-Cre mice carrying the R26R reporter gene at E13.5. Palatal shelves were dissected for the preparation of primary MEPM cells (indicated by yellow dashed lines). Bar, 50 μm. (b) Cell sorting by fluorescein di-β-d-galactopyranoside to detect MEPM cells carrying the R26R reporter gene. Primary MEPM cells derived from the palates of both Tgfbr2^{fl/+}; Wnt1-Cre and Tgfbr2^{fl/fl}; Wnt1-Cre mice are composed of over 93% CNCC-derived cells. (c) Coomassie staining of extracts from primary MEPM cells of Tgfbr2^{fl/fl}, Tgfbr2^{fl/+}; Wnt1-Cre, and Tgfbr2^{fl/fl}; Wnt1-Cre mice. Altered bands were identified by mass spectrometry analyses.

FIG. 18 Altered Smad-independent Tgf-β signaling pathway in Tgfbr2^{fl/fl}; Wnt1-Cre mice. (a) Immunofluorescence (IF) analysis of primary MEPM cells of Tgfbr2^{fl/fl} and Tgfbr2^{fl/fl}; Wnt1-Cre mice using anti-14-3-3ζ/δ or anti-phosphorylated 14-3-3 antibody. Bar, 20 μm. (b) IHC staining of 14-3-3ζ/δ in sections of Tgfbr2^{fl/fl} and Tgfbr2^{fl/fl}; Wnt1-Cre mice at E13.5. 14-3-3ζ/δ expression appears increased in Tgfbr2^{fl/fl}; Wnt1-Cre palate (arrow) compared to Tgfbr2^{fl/fl} littermate control. Bar, 50 μm. (c) IB analysis of indicated molecules in primary MEPM cells from Tgfbr2^{fl/fl} and Tgfbr2^{fl/fl}; Wnt1-Cre mice. Uncropped images of blots are shown in Supplementary information, FIG. 22. (d) IHC staining of phosphorylated p38 in sections of Tgfbr2^{fl/fl} and Tgfbr2^{fl/fl}; Wnt1-Cre mice at E14.0. Box is shown enlarged on the right. Phosphorylated p38 is detectable in palatal mesenchyme of Tgfbr2^{fl/fl}; Wnt1-Cre mice, but not Tgfbr2^{fl/fl} littermate control. Bar, 50 μm. (e) IF analysis of primary MEPM cells of Tgfbr2^{fl/fl} and Tgfbr2^{fl/fl}; Wnt1-Cre mice using anti-β-spectrin antibody. Bar, 50 μm. Inserts show higher magnification, and dotted lines indicate cell surface. (f) IHC staining of β-spectrin in sections of Tgfbr2^{fl/fl} and Tgfbr2^{fl/fl}; Wnt1-Cre mice at E14.0. β-spectrin expression appears increased in Tgfbr2^{fl/fl}; Wnt1-Cre palate compared to Tgfbr2^{fl/fl} littermate control. Bar, 50 μm.

FIG. 19 Altered ligand/receptor assembly in the absence of Tgfbr2. IB analysis of immunoprecipitation (IP) by anti-β-spectrin antibody of extracts from MEPM cells of Tgfbr2^{fl/fl}; Wnt1-Cre mice, treated with Tgf-β1 (10 ng/ml) or Tgf-β2 (10 ng/ml) for 1 hour. Uncropped images of blots are shown in Supplementary information, FIG. 22.

FIG. 20 shows rescued cell proliferation in Tgfbr2^{fl/fl}; Wnt1-Cre palate after the treatment of neutralizing antibody for Tgf-βRIII or Tgf-β2, and p38 MAPK inhibitor. (a) The number of BrdU-labeled nuclei in the palate of Tgfbr2^{fl/fl} (WT) and Tgfbr2^{fl/fl}; Wnt1-Cre (CKO) mice treated with Tgf-β2 neutralizing antibody (b2NA) or IgG control for 24 hours. *, p<0.05. (b) The number of BrdU-labeled nuclei in the palate of Tgfbr2^{fl/fl} (WT) and Tgfbr2^{fl/fl}; Wnt1-Cre (CKO) mice treated with p38 MAPK inhibitor SB203580 (Inhibitor) or vehicle (Veh) control for 24 hours. *, p<0.05. (c) The number of BrdU-labeled nuclei in the palate of Tgfbr2^{fl/fl} (WT) and Tgfbr2^{fl/fl}; Wnt1-Cre (CKO) mice treated with Tgf-βRIII neutralizing antibody (R3NA) or IgG control for 24 hours. *, p<0.05.

FIG. 21 shows the skeletal formation in Tgfbr2^{fl/fl}; Wnt1-Cre; Tgfb2^{fl/fl} and Tgfbr2^{fl/fl}; Wnt1-Cre; Alk5^{fl/fl} newborn mice. (a) Three dimensional microCT images of Tgfbr2^{fl/fl}, Tgfbr2^{fl/fl}; Wnt1-Cre, and Tgfbr2^{fl/fl}; Wnt1-Cre; Tgfb2^{+/-} newborn mice. Open arrow shows clefting, and arrow shows rescued clefting. (b) Slice sections of microCT images of Tgfbr2^{fl/fl}, Tgfbr2^{fl/fl}; Wnt1-Cre, and Tgfbr2^{fl/fl}; Wnt1-Cre; Tgfb2^{+/-} newborn mice. Arrows show fusion of palatine bone, and open arrow shows clefting. (c) Three dimensional microCT images of control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}; Wnt1-Cre; Alk5^{fl/fl} newborn mice. Arrows indicate calvaria defects. P, palatine bone; pp, palatal process of maxilla. (d) Quantitative RT-PCR analyses of Tgfb2 gene in the palate and calvaria of Tgfbr2^{fl/fl} and Tgfbr2^{fl/fl}; Wnt1-Cre mice at E14.5. *, p<0.05.

FIG. 22 shows the uncropped blots.

FIG. 23 Altered Tgf-β signaling pathway in the palate of Tgfbr2^{fl/fl}; Wnt1-Cre mice. (a) Microarray analyses using palates of Tgfbr2^{fl/fl} and Tgfbr2^{fl/fl}; Wnt1-Cre mice at E14.5. Differential expression of Tgfb and Tgfbr family members is listed. ^{a}Ratio of the geometrix means of Tgfbr2^{fl/fl}; Wnt1-Cre and Tgfbr2^{fl/fl} control mice. ^{b}FDR calculated-based on the global microarray analysis (see Methods). *Transcripts showing >1.5-fold change with <5% FDR. (b) Quantitative RT-PCR analyses of indicated genes in the palates of Tgfbr2^{fl/fl} and Tgfbr2^{fl/fl}; Wnt1-Cre mice at E14.5. *, p<0.05 as indicated by a two-tailed Student's t-test. (c) Immunoblotting (IB) analysis of indicated molecules in primary MEPM cells of Tgfbr2^{fl/fl} and Tgfbr2^{fl/fl}; Wnt1-Cre mice. (d) IB analyses of p38 MAPK and 14-3-3ζ/δ in primary MEPM cells of Tgfbr2^{fl/fl} and Tgfbr2^{fl/fl}; Wnt1-Cre mice treated with (+) or without (−) p38 MAPK inhibitor SB203580.

FIG. 24. Altered ligand/receptor assembly and up-regulated phosphorylation and ubiquitination of Tak1 in the absence of Tgfbr2. (a) IB analysis of indicated molecules in primary MEPM cells from Tgfbr2^{fl/fl} and Tgfbr2^{fl/fl}; Wnt1-Cre mice cultured with Tgf-β2 (10 ng/ml) for indicated time. (b) IB analysis of immunoprecipitation (IP) products derived from Tgfbr2^{fl/fl} and Tgfbr2^{fl/fl}; Wnt1-Cre MEPM cell abstracts using the indicated antibodies. (c) Cross-linking analysis after treatment with radioactive Tgf-β in primary MEPM cells from Tgfbr2^{fl/fl} and Tgfbr2^{fl/fl}; Wnt1-Cre mice. (d) IB analysis with anti-ubiquitin (Ub) antibody following IP by anti-Tak1 antibody of extracts from MEPM cells of Tgfbr2^{fl/fl} and Tgfbr2^{fl/fl}; Wnt1-Cre mice. (e) Schematic diagram indicates the mechanism of p38 MAPK activation in Tgfbr2^{fl/fl}; Wnt1-Cre palate. Tgf-β2 is up-regulated and binds to Tgf-βrIII, followed by assembly with Tgf-βrI and β-spectrin. Tak1 is ubiquitinated (U) and phosphorylated (P) following Tgf-β2 binding. Finally, p38 and 14-3-3 proteins are phosphorylated.

FIG. 25. Spontaneous accumulation of lipid droplets in the craniofacial region and primary MEPM cells of Tgfbr2^{fl/fl}; Wnt1-Cre mice. (a) Morphologies of primary MEPM cells from Tgfbr2^{fl/fl} and Tgfbr2^{fl/fl}; Wnt1-Cre mice cultured in regular medium. Lipid droplets were stained with Oil Red O. Bar, 50 μm. (b) Triacylglycerol level in Tgfbr2^{fl/fl} and Tgfbr2^{fl/fl}; Wnt1-Cre MEPM cells cultured for three weeks. Data are expressed as mg per 100 mg protein. ***, P<0.001. (c) Triacylglycerol amounts in newborn Tgfbr2^{fl/fl} and Tgfbr2^{fl/fl}; Wnt1-Cre mice. Data are expressed as mg per 100 mg protein. (d) Oil Red O staining of sections from the craniofacial region of Tgfbr2^{fl/fl} and Tgfbr2^{fl/fl}; Wnt1-Cre mice. Insert shows higher magnification of dotted box. Bar, 50 μm. (e) Electron micrographs of palates from Tgfbr2^{fl/fl} and Tgfbr2^{fl/fl}; Wnt1-Cre mice at E18.5. Bar, 4 μm. Arrow indicates lipid droplet. (f) Oil Red O staining of primary MEPM cells of Tgfbr2^{fl/fl} and Tgfbr2^{fl/fl}; Wnt1-Cre mice treated without (Control) or with p38 MAPK inhibitor SB203580 or PD169316 (4-[5-(4-fluorophenyl)-2-(4-nitrophenyl)-1H-imidazol-4-yl]-pyridine) for three weeks. Lipid droplets did not accumulate in the MEPM cells of Tgfbr2^{fl/fl}; Wnt1-Cre mice with p38 MAPK inhibitor treatment. Bar, 50 μm.

FIG. 26. Rescued cleft palate in Tgfbr2^{fl/fl}; Wnt1-Cre mice via reduction of Tgf-β2 or Tgf-β RI dosage, or treatment with telmisartan. (a) Morphologies of newborn Tgfbr2^{fl/fl} control, Tgfbr2^{fl/fl}; Wnt1-Cre and Tgfbr2^{fl/fl}; Wnt1-Cre; Tgfb2^{+/-} mice. Bottom views show macroscopic appearance of palate at newborn stage. Arrowheads show calvaria defects. Arrow shows cleft palate, and open arrows show normal palates. (b) IB analysis of E14.5 Tgfbr2^{fl/fl}, Tgfbr2^{fl/fl}; Wnt1-Cre, and Tgfbr2^{fl/fl}; Wnt1-Cre; Tgfb2^{+/-} mice. (c) Triacylglycerol amounts in newborn Tgfbr2^{fl/fl}, Tgfbr2^{fl/fl}; Wnt1-Cre, Tgfbr2^{fl/fl}; Wnt1-Cre; Tgfb2^{+/-} mice. Data are expressed as mg per 100 mg protein. (d) Morphologies of newborn control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}; Wnt1-Cre; Alk5^{fl/fl} mice. Bottom views show macroscopic appearance of palate at newborn stage. Arrowheads show calvaria defects. Arrows show cleft palate, and open arrows show normal palates. (e) IB analysis of E14.5 Tgfbr2^{fl/fl}; Wnt1-Cre (lane 1), control (lane 2), Tgfbr2^{fl/fl}; Wnt1-Cre; Alk5^{fl/+} (lane 3), Tgfbr2^{fl/fl}; Wnt1-Cre; Alk5^{fl/fl} (lane 4), and Tgfbr2^{fl/+}; Wnt1-Cre; Alk5^{fl/fl} (lane 5) mice. (f) Lateral, frontal, and palatal views of Tgfbr2^{fl/fl} littermates and Tgfbr2^{fl/fl}; Wnt1-Cre mice after treatment with telmisartan or vehicle. Arrows and dotted line indicate the edges of palate. Arrowheads show defects in the frontal bone. (g) Immunoblotting analysis of indicated molecules in Tgfbr2^{fl/fl} and Tgfbr2^{fl/fl}; Wnt1-Cre mice after treatment with telmisartan (+) or vehicle (−). (h) Triacylglycerol level in Tgfbr2^{fl/fl} and Tgfbr2^{fl/fl}; Wnt1-Cre mice after treatment with telmisartan (+) or vehicle (−) at E18.5. *, P<0.05; NS, not significant.

FIG. 27. Expression of genes relevant to Tgf-β signaling in the palate of Tgfbr2^{fl/fl}; Wnt1-Cre mice. Relationships of up-regulated genes (>1.5-fold, <5% FDR) relevant to Tgf-β signaling in the cleft palate of Tgfbr2^{fl/fl}; Wnt1-Cre mice relative to normal palates of Tgfbr2^{fl/fl} mice at E14.5, as highlighted through Ingenuity Pathway Analysis (IPA) software analysis.

FIG. 28. Up-regulated expression of Tgfb2 in Tgfbr2^{fl/fl}; Wnt1-Cre palate. (a) Quantitative RT-PCR analyses of Tgfb2 genes in the palate and calvaria of Tgfbr2^{fl/fl} and Tgfbr2^{fl/fl}; Wnt1-Cre mice at E14.5. *, p<0.05. (b) Immunoblotting (IB) analysis of E14.5 Tgfbr2^{fl/fl} and Tgfbr2^{fl/fl}; Wnt1-Cre palate.

FIG. 29. Up-regulated expression of Tgf-β2 in Tgfbr2^{fl/fl}; Wnt1-Cre palate. (a, b) Immunohistochemical (IHC) staining of Tgf-β2 in sections of Tgfbr2^{fl/fl} and Tgfbr2^{fl/fl}; Wnt1-Cre mice at E13.5 (a) and E14.0 (b). Tgf-β2 expression is detectable in the palatal mesenchyme of both Tgfbr2^{fl/fl} and Tgfbr2^{fl/fl}; Wnt1-Cre mice, but appears increased in Tgfbr2^{fl/fl}; Wnt1-Cre palate compared to Tgfbr2^{fl/fl} littermate control at E14.0. Arrows show palate. Bar, 50 μm.

FIG. 30. Up-regulated expression of Tgf-β receptor type III in Tgfbr2^{fl/fl}; Wnt1-Cre palate. (a-d) IHC staining of Tgf-βrIII in sections of Tgfbr2^{fl/fl} and Tgfbr2^{fl/fl}; Wnt1-Cre mice at E13.5 (a), E14.0 (b), and E14.5 (c, d). Tgf-βrIII expression is detectable in palatal mesenchyme of both Tgfbr2^{fl/fl} and Tgfbr2^{fl/fl}; Wnt1-Cre mice, but appears increased in Tgfbr2^{fl/fl}; Wnt1-Cre palate compared to Tgfbr2^{fl/fl} littermate control. Arrows show palate. Open arrows in (d) show no positive signal of Tgf-βrIII in the microcartilage and tongue. Bar, 50 μm.

FIG. 31. Isolation of CNCC from Tgfbr2^{fl/fl}; Wnt1-Cre palate. (a) LacZ staining of Wnt1-Cre mice carrying the R26R reporter gene at E13.5. Palatal shelves were dissected for the preparation of primary MEPM cells (indicated by yellow dashed lines). Bar, 50 μm. (b) Cell sorting by fluorescein di-β-d-galactopyranoside to detect MEPM cells carrying the R26R reporter gene. Primary MEPM cells derived from the palates of both Tgfbr2^{fl/+}; Wnt1-Cre and Tgfbr2^{fl/fl}; Wnt1-Cre mice are composed of over 93% CNCC-derived cells.

FIGS. 32A and 32B. Identification of molecules with increased expression in Tgfbr2^{fl/fl}; Wnt1-Cre palate compared to control. (a) Coomassie staining of extracts from primary MEPM cells of Tgfbr2^{fl/fl}, Tgfbr2^{fl/+}; Wnt1-Cre, and Tgfbr2^{fl/fl}; Wnt1-Cre mice. Altered bands were identified by mass spectrometry analyses. (b) Immunofluorescence (IF) analysis of primary MEPM cells of Tgfbr2^{fl/fl} and Tgfbr2^{fl/fl}; Wnt1-Cre mice using anti-14-3-3ζ/δ or anti-phosphorylated 14-3-3 antibody. Bar, 20 μm. (c) IHC staining of 14-3-3ζ/δ in sections of Tgfbr2^{fl/fl} and Tgfbr2^{fl/fl}; Wnt1-Cre mice at E13.5. 14-3-3ζ/δ expression appears increased in Tgfbr2^{fl/fl}; Wnt1-Cre palate (arrows) compared to Tgfbr2^{fl/fl} littermate control. Bar, 50 μm. (d) IF analysis of primary MEPM cells of Tgfbr2^{fl/fl} and Tgfbr2^{fl/fl}; Wnt1-Cre mice using anti-β-spectrin antibody. Bar, 50 μm. Inserts show higher magnification. (e) IHC staining of β-spectrin in sections of Tgfbr2^{fl/fl} and Tgfbr2^{fl/fl}; Wnt1-Cre mice at E14.0. β-spectrin expression appears increased in Tgfbr2^{fl/fl}; Wnt1-Cre palate compared to Tgfbr2^{fl/fl} littermate control. Bar, 50 μm. (f) IB analysis of indicated molecules in primary MEPM cells from Tgfbr2^{fl/fl} and Tgfbr2^{fl/fl}; Wnt1-Cre mice. (g) IHC staining of phosphorylated p38 in sections of Tgfbr2^{fl/fl} and Tgfbr2^{fl/fl}; Wnt1-Cre mice at E14.0. Phosphorylated p38 is detectable in palatal mesenchyme of Tgfbr2^{fl/fl}; Wnt1-Cre mice, but not Tgfbr2^{fl/fl} littermate control. Bar, 50 μm.

FIG. 33. Altered ligand/receptor assembly in the absence of Tgfbr2. IB analysis of immunoprecipitation (IP) by anti-β-spectrin antibody of extracts from MEPM cells of Tgfbr2^{fl/fl} and Tgfbr2^{fl/fl}; Wnt1-Cre mice, treated with Tgf-β1 (10 ng/ml) or Tgf-β2 (10 ng/ml) for 1 hour.

FIG. 34. Lipid droplet accumulation in Tgfbr2^{fl/fl}; Wnt1-Cre cells. (a) Quantitative analysis of lipid accumulation by Oil Red O staining in Tgfbr2^{fl/fl} and Tgfbr2^{fl/fl}; Wnt1-Cre MEPM cells. Data are OD490 measurements. ***, P<0.001. (b) IB analysis of molecules related to Akt signaling in the MEPM cells of Tgfbr2^{fl/fl} and Tgfbr2^{fl/fl}; Wnt1-Cre mice. Both insulin signaling and glucose uptake have been previously shown to have a secondary effect on lipid metabolism via activation of the Akt signaling pathway^{42, 43}. There were no changes in Akt signaling in Tgfbr2^{fl/fl}; Wnt1-Cre MEPM cells compared to the control. (c) Pulse-chase analyses of lipid droplet formation using chemical inducers of lipid droplet-synthesis and lipolysis. The number of lipid droplets increased in MEPM cells of both Tgfbr2^{fl/fl} and Tgfbr2^{fl/fl}; Wnt1-Cre mice after inducing lipogenesis with oleic acid. The number of lipid droplets decreased immediately in Tgfbr2^{fl/fl} MEPM cells after we switched to a medium that included isoproterenol, a chemical inducer of lipolysis. In contrast, lipid droplet number remained high after the switch to isopreterenol-containing medium in Tgfbr2^{fl/fl}; Wnt1-Cre MEPM cells, suggesting that Tgfbr2^{fl/fl}; Wnt1-Cre MEPM cells had a defect in lipolysis. **, P<0.01. (d) Quantitative analysis of adipogenesis by Oil Red O staining in Tgfbr2^{fl/fl} and Tgfbr2^{fl/fl}; Wnt1-Cre MEPM cells after one week culture in adipogenic induction medium (+) or regular medium (−). Data are relative OD490 measurements. **, P<0.01. (e) Quantitative analysis of glycerol released into the medium by isoproterenol stimulation (+) or mock treatment (−) in MEPM-derived adipocytes of Tgfbr2^{fl/fl} and Tgfbr2^{fl/fl}; Wnt1-Cre mice. *, P<0.05; **, P<0.01; NS, not significant. (f, g) Quantitative RT-PCR analyses of genes related to adipocyte differentiation in the palate of Tgfbr2^{fl/fl} and Tgfbr^{fl/fl}; Wnt1-Cre mice at E13.5 (f) and E14.5 (g). Pparg, peroxisome proliferator-activated receptor gamma; Fabp4, fatty acid binding protein 4. *, P<0.05. (h) Electron micrographs of palatal mesenchyme of Tgfbr2^{fl/fl} and Tgfbr2^{fl/fl}; Wnt1-Cre mice at E14.5. Arrow indicates lipid droplet accumulation. Bar, 5 μm. (i) Oil Red O staining of Tgfbr2^{fl/fl} and Tgfbr2^{fl/fl}; Wnt1-Cre MEPM cells after culturing for three weeks with neutralizing antibodies against Tgf-βrI (TβrI), Tgf-βrII (TβrII), Tgf-βrIII (TβrIII), and Tgf-β2 and control IgG. Neutralizing antibodies against Tgf-β2, Tgf-βrI, and Tgf-βrIII block the spontaneous accumulation of lipid droplets in Tgfbr2^{fl/fl}; Wnt1-Cre MEPM cells. Bar, 50 μm.

FIG. 35. Rescued cleft palate in Tgfbr2^{fl/fl}; Wnt1-Cre mice via reduction of Tgf-β2 dosage. (a) Morphologies of E16.5 Tgfbr2^{fl/fl} control, Tgfbr2^{fl/fl}; Wnt1-Cre, and Tgfbr2^{fl/fl}; Wnt1-Cre; Tgfb2^{+/-} mice. Bottom views show macroscopic appearance of palate at E16.5. Arrowheads show calvaria defects. Arrow shows cleft palate, and open arrows show normal palates. (b) Whole-mount skeletal staining with Alcian blue-Alizarin Red S of Tgfbr2^{fl/fl}, Tgfbr2^{fl/fl}; Wnt1-Cre, and Tgfbr2^{fl/fl}; Wnt1-Cre ; Tgfb2^{+/-} newborn mice. (c) Slice sections of microCT images of Tgfbr2^{fl/fl}, Tgfbr2^{fl/fl}; Wnt1-Cre, and Tgfbr2^{fl/fl}; Wnt1-Cre; Tgfb2^{+/-} newborn mice. Arrows show fusions of palatine bone, and open arrow shows clefting. (d) Three dimensional microCT images of Tgfbr2^{fl/fl}, Tgfbr2^{fl/fl}; Wnt1-Cre, and Tgfbr2^{fl/fl}; Wnt1-Cre; Tgfb2^{+/-} newborn mice. Open arrow shows clefting, and arrow shows rescued clefting. (e) BrdU staining of Tgfbr2^{fl/fl}, Tgfbr2^{fl/fl}; Wnt1-Cre, and Tgfbr2^{fl/fl}; Wnt1-Cre; Tgfb2^{+/-} mice at E14.0. Most Tgfbr2^{fl/fl}; Wnt1-Cre; Tgfb2^{+/-} mice show a half-day delay in the elevation of palatal shelves, although some Tgfbr2^{fl/fl}; Wnt1-Cre; Tgfb2^{+/-} palates are indistinguishable from wild-type littermate palates at E14.0. Bar, 50 μm. (f) The number of BrdU-labeled nuclei in the palate of Tgfbr2^{fl/fl}; Tgfbr2^{fl/fl}; Wnt1-Cre, and Tgfbr2^{fl/fl}; Wnt1-Cre; Tgfb2^{+/-} mice at E14.0. ***, p<0.001.

FIG. 36. Rescued cleft palate in Tgfbr2^{fl/fl}; Wnt1-Cre mice via reduction of Tgf-β2 dosage. Hematoxylin and eosin staining of sections of Tgfbr2^{fl/fl}, Tgfbr2^{fl/fl}; Wnt1-Cre, and Tgfbr2^{fl/fl}; Wnt1-Cre; Tgfb2^{+/-} mice at E14.0, E14.5, and E16.5. Arrows indicate palate. Bar, 50 μm.

FIG. 37. Rescued cleft palate in Tgfbr2^{fl/fl}; Wnt1-Cre mice via reduction of Tgf-β receptor type I dosage. (a) Hematoxylin and eosin staining of sections of control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}; Wnt1-Cre; Alk5^{fl/fl} mice at E14.0 and E14.5. Most Tgfbr2^{fl/fl}; Wnt1-Cre; Alk5^{fl/+} mice show a half-day delay in the elevation of palatal shelves, although some Tgfbr2^{fl/fl}; Wnt1-Cre; Alk5^{fl/+} palates are indistinguishable from wild-type littermate palates at E14.0. Arrows show palate. Bar, 50 μm. (b) BrdU staining of control, Tgfbr2^{fl/fl}; Wnt1-Cre, and Tgfbr2^{fl/fl}; Wnt1-Cre; Alk5^{fl/+} mice at E14.0. Bar, 50 μm. (c) The number of BrdU-labeled nuclei in the palate of control, Tgfbr2^{fl/fl}; Wnt1-Cre, and Tgfbr2^{fl/fl}; Wnt1-Cre; Alk5^{fl/+} mice at E14.0. p<0.001. (d) Three dimensional microCT images of control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}; Wnt1-Cre; Alk5^{fl/fl} newborn mice. (e) Whole-mount skeletal staining with Alcian blue-Alizarin Red S of control, Tgfbr2^{fl/fl}; Wnt1-Cre; Alk5^{fl/+}, Tgfbr2^{fl/fl}; Wnt1-Cre; Alk5^{fl/fl}, and Tgfbr2^{fl/+}; Wnt1-Cre; Alk5^{fl/fl} newborn mice.

FIG. 38. Up-regulated angiotensin-Tgf-β signaling in Tgfbr2^{fl/fl}; Wnt1-Cre mice. Schematic diagram of angiotensin-Tgf-β signaling. Selected genes are listed from the E14.5 microarray analysis of Tgfbr2^{fl/fl} and Tgfbr2^{fl/fl}; Wnt1-Cre mice. *, p<0.05; **, p<0.005.

FIG. 39. Histological analyses of embryos after feeding of telmisartan to pregnant females. (a) Histology of Tgfbr2^{fl/fl} littermates and Tgfbr2^{fl/fl}; Wnt1-Cre mice after treatment with telmisartan or vehicle. Arrows indicate the palate. Lower panels show higher magnification of upper panels. (b) Morphology (left panel A, Tgfbr2^{fl/fl}; Wnt1-Cre heart) and histology (right panels) of the heart of Tgfbr2^{fl/fl} and Tgfbr2^{fl/fl}; Wnt1-Cre mice after treatment with telmisartan. Telmisartan does not rescue the heart defect in Tgfbr2^{fl/fl}; Wnt1-Cre mice. (c) Histology of Tgfbr2^{fl/fl} littermates and Tgfbr2^{fl/fl}; Wnt1-Cre mice after treatment with telmisartan or vehicle. Telmisartan does not appear to affect kidney or liver in Tgfbr2^{fl/fl} and Tgfbr2^{fl/fl}; Wnt1-Cre embryos.

### DETAILED DESCRIPTION

To facilitate a full and complete understanding of the present invention, the following theoretical discussion is offered. It is to be understood that the following discussion is solely for illustrative purposes only and not intended to be limiting the scope of the present invention.

### 1. Roles of Dxl5, Shh, and Msx1 in Oral-Nasal Patterning

The mammalian palate develops from two primordia: the primary palate and the secondary palate. The primary palate represents only a small part of the adult hard palate. The secondary palate is the primordium for most of the hard and all of the soft parts of the palate. Palate development is a multi-step process that involves palatal shelf growth, elevation, midline fusion of palatal shelves and the disappearance of the midline epithelial seam. The palatal structures are composed of the cranial neural crest (CNC)-derived ectomesenchyme and pharyngeal ectoderm (Ferguson, 1988; Shuler, 1995). Throughout palatal development, there is continuous epithelial-mesenchymal interaction that is essential for the growth and fusion of the palate. The most common type of cleft palate documented in animal studies, which also most closely resembles cleft palate in humans, is the failure of palate shelf expansion following elevation (Chai and Maxson, 2006; Ito et al., 2003; Rice et al., 2004; Satokata and Maas, 1994).

In the developing palate, the epithelia that cover the palatal shelves are divided into oral, nasal, and medial edge epithelium (Chai and Maxson, 2006). The nasal and oral epithelia differentiate into pseudostratified and squamous epithelia, respectively, whereas the medial edge epithelium (MEE) is removed from the fusion line by means of programmed cell death and cell migration (Martinez-Alvarez et al., 2000; Vaziri Sani et al., 2005). The palatal mesenchyme is mainly derived from CNC cells (Ito et al., 2003) and has been treated as a homogeneous population in previous studies. The oral-nasal patterning of the palatal mesenchyme and the molecular regulation of the fate of mesenchymal cells must be taken into account when analyzing palatal development.

Dlx5, Shh, and Msx1 control the fate of CNC cells. Specifically, Dlx5 plays a critical role in regulating the patterning of craniofacial structures (Depew et al., 1999; Qiu et al., 1997; Yang et al., 1998). Nested Dlx gene expression in the branchial arches patterns proximodistal axes and is crucial in the acquisition and refinement of mammalian jaws through evolution (Depew et al., 2002). Sonic hedgehog (Shh) mediates the ventral inductive signaling during the dorsal-ventral patterning of the spinal cord (Jessell, 2000). Within the CNC population, Shh is required for cardiac outflow tract and facial primordial development via regulation of CNC cell survival and proliferation (Jeong et al., 2004; Washington Smoak et al., 2005). During palatogenesis, Shh expression is restricted to the oral side of the palatal epithelium, and conditional inactivation of Shh in the ectoderm leads to dramatic shortening of the palatal shelves and cleft palate (Lan and Jiang, 2009; Rice et al., 2004). Exogenous Shh stimulates palatal mesenchyme proliferation in palatal explant culture (Bei et al., 2000). Interestingly, a recent study shows that overexpression of Shh signaling in the palatal ectoderm also leads to cleft palate (Cobourne et al., 2009). Collectively, these studies suggest that Shh signaling needs to be tightly regulated during palatogenesis.

Msx1 is critical for the development of palate, teeth and other craniofacial structures (Han et al., 2003; Satokata and Maas, 1994). In humans, mutations in the MSX1 gene result in orofacial clefting and tooth agenesis, consistent with the phenotype observed in Msx1 mutant mice (Hu et al., 1998; Jumlongras et al., 2001; van den Boogaard et al., 2000; Vastardis et al., 1996). In mice, Msx1 is required for Bmp4 and Bmp2 expression in the palatal mesenchyme and Shh expression in the palatal epithelium. Shh acts downstream of Bmp4 and upstream of Bmp2 to stimulate mesenchymal cell proliferation to promote the outgrowth of the palatal shelf (Zhang et al., 2002).

In the present invention, we have investigated the establishment of O—N patterning in the palate by assaying the expression of various asymmetric gene markers and investigating the palatal phenotype associated with the loss of Dlx5 in mice. We find that O—N patterning is associated with the expansion and fusion of the palatal shelves and that Dlx5 is required in the O—N patterning of palatal mesenchyme. Dlx5 is specifically required for Fgf7 expression in the nasal side of palatal mesenchyme. Furthermore, FGF7 strongly inhibits Shh expression in the nasal side of palatal shelf epithelium. Loss of Dlx5 results in downregulation of Fgf7 and an expansion of Shh expression into the nasal side of the palatal epithelium. This expanded Shh signaling is sufficient to rescue palatal fusion, as Msx1/Dlx5 double null mutant mice show restored CNC cell proliferation and palate fusion. Furthermore, Msx1 and Dlx5 antagonistically regulate the expression of Shh, which in turn controls the fate of CNC cells through tissue-tissue interaction during palatogenesis. Finally, we report that Dlx5 is critical for the patterning of soft palate.

### 2. Novel TGF-β Signaling Mechanism in Craniofacial Formation

Mutations in TGFBR1 or TGFBR2 are associated with Loeys-Dietz syndrome (previously called Marfan syndrome type II) in humans, which can manifest with craniofacial malformations such as cleft palate, craniosynostosis, hypertelorism, and vascular defects. In addition, mutations in FBN-1, which encodes an elastic extracellular matrix protein called fibrillin-1, lead to excessive Tgf-β signaling and cause Marfan syndrome, which exhibits clinical phenotypes similar to Loeys-Dietz syndrome. Furthermore, DiGeorge syndrome, which results from a variably sized deletion on chromosome 22 (del22q11) exhibits altered TGF-β signaling in approximately 80% of the patients. Thus, TGF-β signaling is implicated in regulating craniofacial development and altered TGF-β signaling lead to multiple malformations in humans.

Tgf-β transmits signals through a membrane receptor serine/threonine kinase complex that phosphorylates Smad2 and Smad3, followed by the formation of transcriptional complexes with Smad4 and translocation into the nucleus. In addition, Smad-independent pathways transduce Tgf-β signals in some physiological and pathological conditions. Tgf-β ligands target a variety of genes in a developmental stage-dependent and cell type-specific manner, but it was unknown how a Smad-dependent versus independent pathway is initiated and specified, and whether mutations in Tgfbr genes causes loss- or gain-of-function with respect to signaling.

Previous studies suggested that Tgf-βRIII appears dispensable for Tgf-β-mediated signal transduction because it lacks an intracellular domain and most cells that lack functional Tgf-βRIII still respond to Tgf-β. However, previous studies failed to consider how Tgf-β signals in the absence of Tgf-βRII. Without being limited to theory, in the present invention, we show here that a Tgf-βRIII/Tgf-βRI/β-spectrin complex can be activated by elevated Tgf-β2 and utilizes the p38 MAPK signaling cascade to regulate downstream target genes. Thus, it is a surprising discovery of the present invention that elevated Tgf-β2 activity and a resulting ectopic p38 MAPK activation is responsible for adversely affecting cell proliferation in the CNCC-derived palatal mesenchyme.

### 3. Lipid Metabolism and Craniofacial Malformation

Inborn errors of lipid metabolism also cause human malformation syndromes, such as Smith-Lemli-Opitz syndrome, lathosterolosis, desmosterolosis, X-linked dominant chondrodysplasia punctata type 2, congenital hemidysplasia with ichthyosiform erythroderma and limb defects. All of these syndromes involve craniofacial anomalies including cleft palate. Moreover, animal models for a cholesterol synthesis defect, such as Dhcr7, Sc5d, and Insig-1/-2 null mutant mice, have cleft palate. Interestingly, these mice have severe malformations specifically in the craniofacial region. Because the majority of cells in the craniofacial region are derived from cranial neural crest cells (CNCC), these results suggest that CNCC are more sensitive to lipid metabolic aberrations than are cells from other regions during embryogenesis.

However, it was unknown how molecules related to lipid metabolism are regulated in CNCC during craniofacial development and what are the relationships between lipid metabolic aberrations and craniofacial abnormalities.

Here we show that loss of Tgfbr2 in cranial neural crest cells (CNCC) in mice results in elevated Tgf-β2 and Tgf-βRIII expressions, activation of a novel Tgf-β type I/III receptor-mediated p38 MAPK/14-3-3 signaling pathway, lipid metabolic aberrations, and a cell proliferation defect in the palatal mesenchyme. Strikingly, Tgfb2 or Tgfbr1/Alk5 haploinsufficiency restores lipid metabolic activity and cell proliferation in CNCC and rescues craniofacial deformities in Tgfbr2 mutant mice. Furthermore, treatment of pregnant mice with telmisartan improves lipid metabolism and prevents the cleft palate in Tgfbr2 mutant mice. Modulation of TGF-β signaling may thus be therapeutically beneficial for the prevention and treatment of congenital birth defects.

It is a surprising discovery of the present invention that elevated Smad-independent p38 MAPK activation mediated through Tgf-βRI/RIII is responsible for the CNCC proliferation defect and failure of palatal fusion.

It is also a surprising discovery of the present invention that reduction of up-regulated p38 MAPK activity and improvement of lipid metabolism restored cell proliferation defect and rescued cleft palate in about 30% of the Tgfbr2^{fl/fl}; Wnt1-Cre mice following telmisartan treatment. Loss of Tgfbr2 causes lipid metabolic aberrations that adversely affect cell proliferation during palatogenesis, and importantly medications that affect lipid metabolism can be useful for the prevention of craniofacial anomalies during embryogenesis.

It is a further discovery of the present invention that elevated Tgf-β2 expression in Tgfbr2^{fl/fl}; Wnt1-Cre; Alk5^{fl/+} mice activates a Smad-independent Tgf-β signaling cascade and causes lipid accumulation and adversely affects CNCC proliferation during palatogenesis. A reduction of Tgfbr1 corrects the lipid metabolic aberrations, restores cell proliferation, and rescues the cleft palate as shown in phenotype in Tgfbr2^{fl/fl}; Wnt1-Cre; Alk5^{fl/+} mice. Our findings indicate that elevated Smad-independent p38 MAPK activation mediated through Tgf-βRI/RIII is responsible for the CNCC proliferation defect and failure of palatal fusion.

To further illustrate the various discoveries, methods, and compositions of the present invention, the following specific examples are provided. It will be undersood that although the present invention has been described in terms of specific exemplary embodiments and examples, the embodiments disclosed herein are for illustrative purposes only and various modifications and alterations might be made by those skilled in the art without departing from the spirit and scope of the invention as set forth in the following claims.

### EXAMPLES

### Example 1

### The Roles of Dxl5, Shh, and Msx1 in Oral-Nasal Patterning

### Materials and Methods

Mutant mice, histological and skeleton analysis and scanning electron microscope (SEM). Mice carrying Msx1+/− and Dlx5+/− alleles have been described previously (Depew et al., 1999; Satokata and Maas, 1994). We crossed Msx1+/−; Dlx5+/− mice to generate Msx1/Dlx5 double null mutants. All samples were fixed in 10% buffered formalin and processed through serial ethanol, and paraffin embedded and sectioned using routine procedures. For general morphology, deparaffinized sections were stained with Hematoxylin and Eosin using standard procedures. Skeletal structures were stained using Alcian Blue for non-mineralized cartilage and Alizarin Red for bone, as described previously (Ito et al., 2003). For SEM, samples were fixed with 10% buffered formalin at 4° C. overnight. After dehydration through a graded ethanol series, samples were trimmed and dried in a Balzer Union (FL-9496) apparatus, and coated with colloidal silver liquid (Ted Pella Inc.) by a Technics Hummer V sputter coater. Samples were examined with a Cambridge 360 scanning electron microscope.

Palatal shelf organ cultures and bead implantation. Timed-pregnant mice were sacrificed on postcoital day 13.5 (E13.5). Genotyping was carried out as previously described (Depew et al., 1999; Satokata and Maas, 1994). Paired secondary palatal shelves were microdissected and cultured in serumless, chemically-defined medium as previously described (Ito et al., 2003). For bead implantation, Affi-Gel blue agarose beads (BioRad) were soaked in proteins as previously described (Zhang et al., 2002). Tissues were harvested after 24 hours of culture and fixed in 4% paraformaldehyde for processing. Shh N-terminal peptide (R & D 7 systems) was used at 1 mg/ml, anti-Shh antibody (Developmental Studies Hybridoma Bank) was used at 0.30 mg/ml, and BSA was used at 10 ng/ml. Neutralizing antibodies to Fgf7 (MAB251) and Mouse IgG1 (MAB002) (R & D Systems) were added to the culture medium at concentrations of 50 μg/ml.

Apoptosis and cell proliferation. Following treatment with 20 mg/ml proteinase K for 15 minutes at room temperature, apoptotic cells were assayed by the TUNEL procedure using the In Situ Cell Death Detection (fluorescein) kit (Roche Molecular Biochemicals) by following the manufacturer's protocol. Cell proliferation was scored by injection of BrdU (5-bromo-2′-deoxy-uridine, Sigma. 100 μg/g body weight) into pregnant females 1 hour before recovery of embryos. For palatal organ culture, BrdU was supplemented into culture medium at concentration of 100 μM for 2 hours before harvest. Detection of BrdU labeled cells was carried out using a BrdU Labeling and Detection kit following the manufacturer's protocol (Zymed).

In situ hybridization. Samples for whole-mount and section in situ hybridization were fixed in freshly made 4% paraformaldehyde/PBS. In situ hybridizations were performed as previously described (Xu et al., 2005). The following cDNAs were used to generate antisense riboprobes: a 1.2 kb fragment of mouse Msx1, a 900 bp mouse Dlx5, a 1.6 kb mouse Gli1, and 640 bp mouse Shh. Non-radioactive RNA probes were generated by in vitro transcription labeling with digoxigenin-UTP according to the manufacturer's protocol (Roche Molecular Biochemicals).

Real-time PCR. E13.5 palatal shelves (six wild type and six mutants, in two independent experiments) were precisely dissected. Total RNA was extracted using the RNeasy kit (Qiagen, USA). Quality of RNA, primer efficiency and correct size were tested by RT-PCR. RealTime PCR was performed with Cycler (Bio-rad) using IQ SYBR-Green (Bio-rad). 18S RNA was used to normalization.

Primer sequences are:

SEQ ID No. 1:
18S 5′-CGGCTACCACATCCAAGGAA-3′
SEQ ID No. 2:
18S AS 5′-GCTGGAATTACCGCGGCT-3′
SEQ ID No. 3:
Fgf7 F 5′-CTCTACAGGTCATGCTTCCACC-3′
SEQ ID No. 4:
Fgf7 R 5′-ACAGAACAGTCTTCTCACCCT-3′

### Results

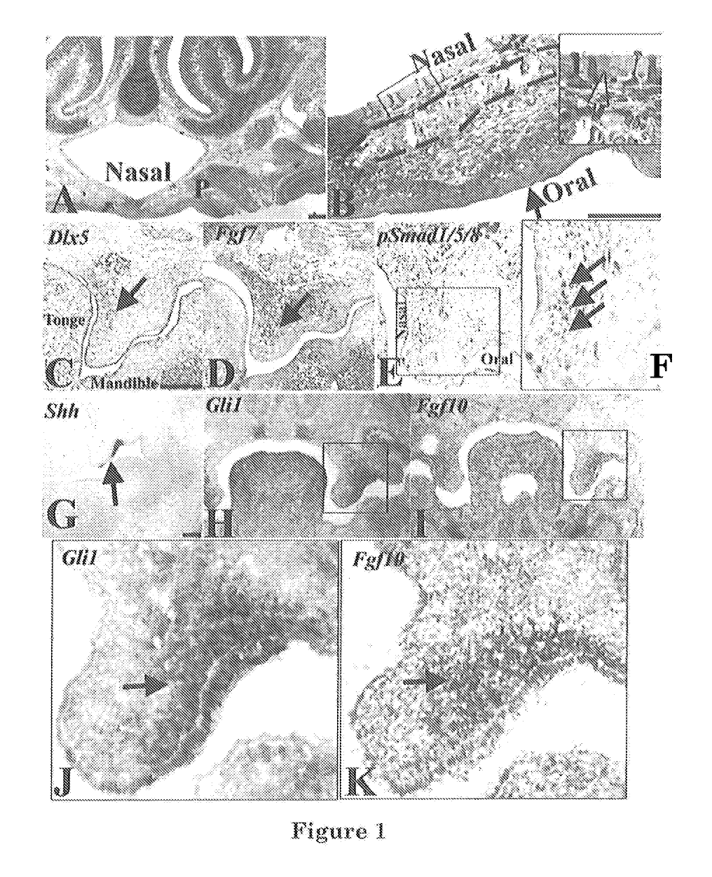

Oral-nasal patterning of mouse palatal mesenchyme. The palatal epithelia is heterogeneous in newborn mice, with pseudostratified, ciliated, columnar epithelia covering the nasal side of the palatal shelf and stratified keratinizing, squamous epithelia covering the oral side (FIGS. 1A,B). Mesenchymal heterogeneity of the nasal and oral regions of the palate is subtle, with the palatine bone in the nasal region and soft tissue in the oral region (FIG. 1B). We hypothesized that these morphological differences are likely the result of molecular heterogeneity established earlier along the oral-nasal axis of developing palate. In fact, Dlx5, Fgf7 and phospho-Smad1/5/8 expression is restricted to the nasal region at E13.5 (FIG. 1C-F), whereas GUI and Fgf10 are expressed mainly in the oral region of the palatal mesenchyme (FIG. 1H-K). Shh is only expressed in the oral side of palatal epithelium at E13.5 (FIG. 1G).

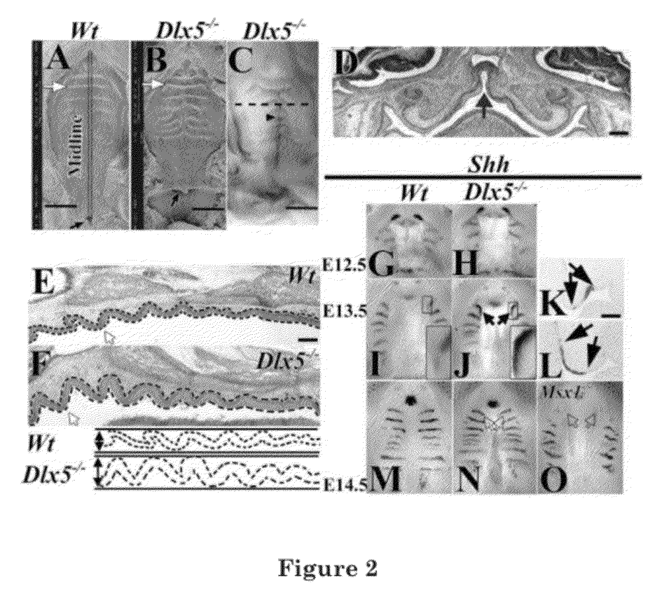

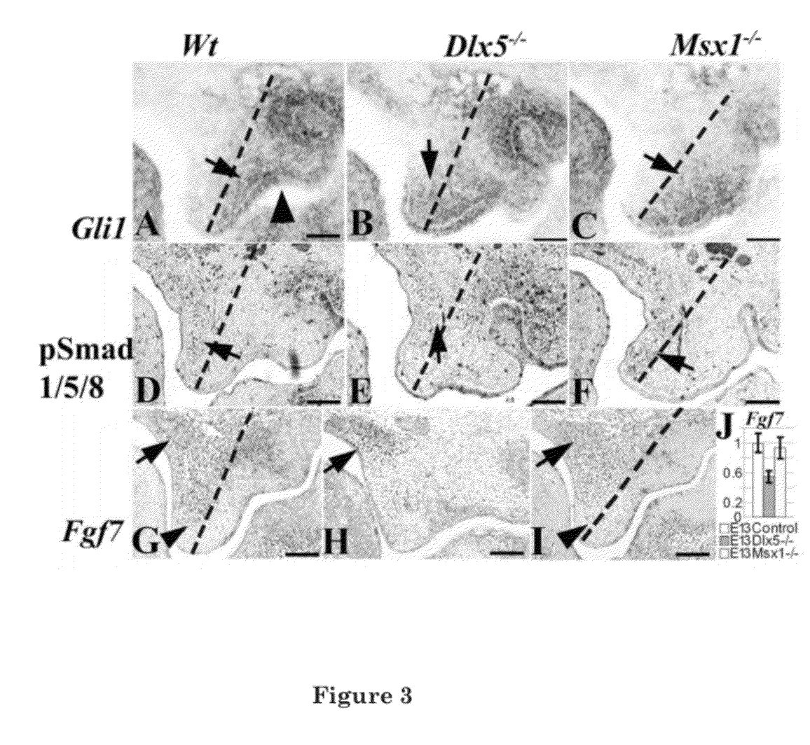

Inactivation of Dlx5 leads to an expansion of the oral region of the palatal shelf. Previous studies examining Dlx5−/− mice were based on skeletal staining, which revealed incomplete palatal bone fusion (Depew et al., 1999; Levi et al., 2006). In order to characterize the palatal phenotype thoroughly, we performed SEM on Dlx5−/− mice. In the anterior region of the palate, the soft tissue was fused in all Dlx5−/− mice (n=45) (FIG. 2A-D). Although some Dlx5−/− mice (n=5, 11%) were born with a groove (a folding of the palatal shelf) in the palate (FIG. 2C), histological analysis clearly revealed that there was normal soft tissue connection between the two palatal shelves (FIG. 2D). The landmark of the oral palatal epithelium, the rugae, are detectable in SEM images of the oral side of newborn palatal shelves (FIGS. 2A,B white arrows). Compared to wild type litter mates, the rugae in Dlx5−/− mice appear more prominent, both in the SEM image (FIGS. 2A,B) and the H & E staining of sagittal sections through the rugae (FIGS. 2E,F). We detected approximately a 40% increase in the height of the ruga and 40% increase in the thickness of the squamous epithelium in Dlx5−/− mutants relative to wild type (FIG. 2F). We did not detect any difference in stratification of the oral side of the palatal epithelium in Dlx5−/− mutants. In contrast, the thickness of the nasal side palatal epithelium in Dlx5−/− mice is comparable to that of the wild type control (FIG. 9). The expansion of the oral side of the palate shelf is most obvious in Dlx5−/− mutants with excencephaly (representing 11% of Dlx5−/− mice), in which the oral palatal epithelium protrudes into the palate forming a groove (FIG. 2C). The expansion of the oral side of the palate shelf in Dlx5−/− mice can be detected at E13.5 based on the expansion of Shh expression into the MEE and nasal side of the palatal epithelium (FIG. 2I-L). This is an expansion, not a shift, as the oral side Shh expression persists (FIG. 2J). In wild type mice, Shh is not expressed in the MEE and nasal side of the palatal epithelium. We confirmed previous studies that reported diminished Shh expression in the anterior part of the oral palatal epithelium in Msx1−/− mutant mice (FIG. 2O) (Zhang et al., 2002). We also found that the expression of Gli1, a key hedgehog (Hh) pathway target (Hooper and Scott, 2005; Lum and Beachy, 2004), expanded from the oral region of the palate into the nasal region in Dlx5−/− mice (FIGS. 3A,B). Phospho-Smad1/5/8, and Fgf7 were detectable in the nasal region of the palatal mesenchyme in wild type mice, but their expression was reduced in Dlx5−/− mutants (FIGS. 3D,E,G,H,J). Our quantitative PCR analysis confirmed greater than 40% reduction in Fgf7 expression in the palate of Dlx5−/− mice (FIG. 3J). Although the expression of Shh is diminished in the palatal epithelium of Msx1−/− mice, the expression of Gli1, phospho-Smad1/5/8 and Fgf7 were indistinguishable from the wild type control (FIGS. 3C,F,I).

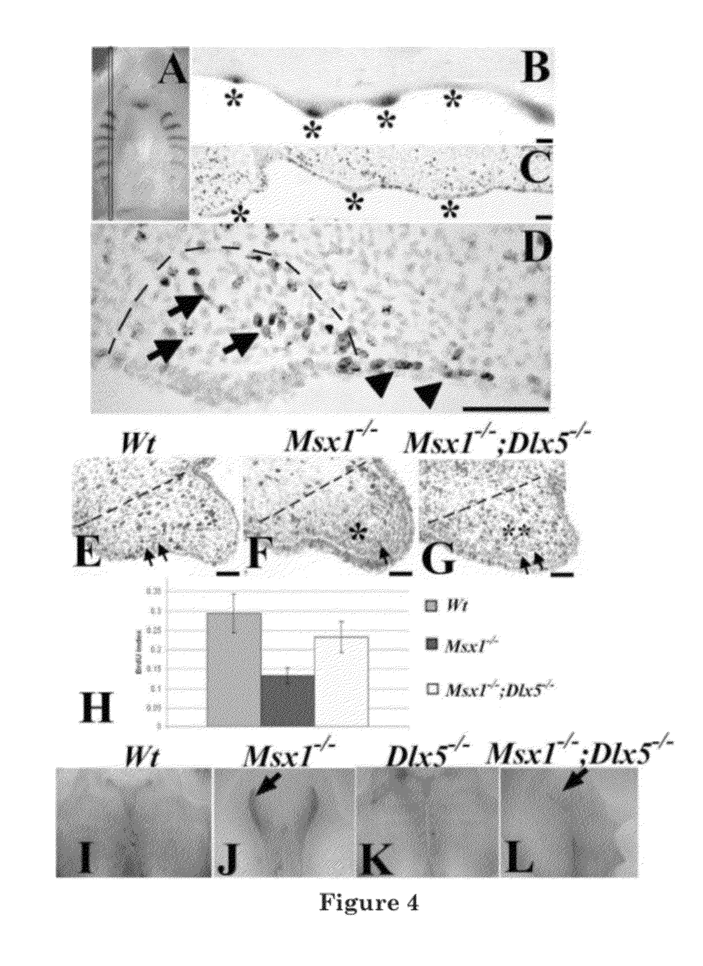

Dlx5 inactivation rescues palatal fusion defects in Msx1 null mice. During palatogenesis, Shh signaling from the oral region of the palatal epithelium is required for palatal mesenchymal proliferation (Rice et al., 2004; Zhang et al., 2002). At E13.5, the expression of Shh in the palatal epithelium is restricted to the epithelial thickening, the developing ruga (FIG. 4A). In sagittal sections, epithelial cells that express Shh are not actively proliferating, whereas the mesenchymal cells underlying the Shh-expressing cells are actively proliferating and show a higher proliferative activity than neighboring regions of mesenchymal cells underlying non-SM-expressing epithelia (FIG. 4B-D). We hypothesized that expanded Shh signaling in the absence of Dlx5 might rescue the palatal mesenchymal proliferation defect in Msx1−/− mutant mice, a cleft palate model in which the two palatal shelves fail to meet at the midline following elevation. By comparing cell proliferation activities in wild type, Msx1−/−, and Msx1−/−; Dlx5−/− palatal mesenchyme (FIG. 4E-H), we found that inactivation of Dlx5 significantly stimulated palatal mesenchymal proliferation in the background of Msx1 null mutation. At E14.0, Msx1−/− mice showed significantly reduced cell proliferation (13.2±2.3%) in the palatal mesenchyme as compared to the control (29.4±5.1%) (FIGS. 4E,F,H). Significantly, CNC cell proliferation activity was restored (23.6±3.8%) in the palatal mesenchyme of Msx1−/−; Dlx5−/− mutants (FIGS. 4G,H). We did not detect altered cell proliferation activity in Dlx5 null mutants compared to wild type littermates (data not shown). There was no difference in apoptotic activity in the palatal mesenchyme of wild type, Msx1−/− or Msx1−/−; Dlx5−/− mutant samples (data not shown). The two palatal shelves were able to reach the midline in Msx1−/−; Dlx5−/− mutants at E14.0 (FIG. 4L), likely as a consequence of the rescued palatal mesenchymal proliferation. As expected, the anterior part of the secondary palatal shelf showed insufficient expansion towards the midline in Msx1−/− mutants (FIG. 4J). The expansion of the palatal shelves in Dlx5−/− mutants was comparable to wild type (FIGS. 4K,I).

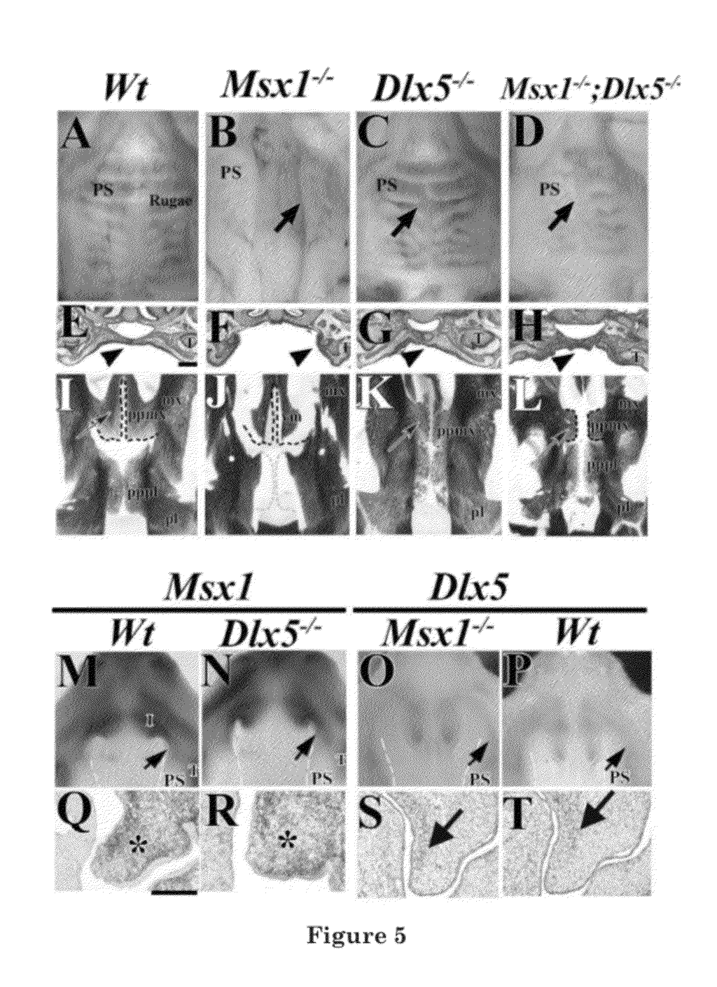

To investigate whether restored cell proliferation in the palatal mesenchyme and palatal shelf extension was sufficient to restore proper palatal fusion in the Msx1 mutant, we examined palatogenesis in Msx1−/−; Dlx5−/− mice. We found that there was complete rescue of the Msx1−/− cleft palate defect in Msx1−/−; Dlx5−/− mutant mice (n=15) (FIG. 5A-D). This rescued palate development required the complete absence of Dlx5, as Msx1−/−; Dlx5+/− palates failed to fuse (data not shown). At E17.5, Msx1−/− mice showed a complete cleft of the secondary palate, the palatal shelves failed to meet at the midline (FIGS. 5B,F) and the palatine processes of the maxilla and of the palatine bones were missing, leaving the vomer visible in an oral view (FIG. 5J). In the Dlx5−/− sample, fusion of the anterior palate was indistinguishable from that of the wild type control; the soft tissue covering the anterior part of the palate was fused completely (FIGS. 5C,G), and the bony parts of the hard palate are present (FIG. 5K). In Msx1−/−; Dlx5−/− mutant mice, palatal fusion was rescued, with confluence of the mesenchyme and reappearance of the palatal bones that were missing in the Msx1−/− mutant (FIGS. 5D,H,L).

Next, we examined whether Msx1 and Dlx5 may regulate each other's expression in an upstream or downstream manner by determining if Msx1 expression was altered in Dlx5−/− mice or vice versa. Msx1 is expressed in the anterior part of the developing palate at E13.5. In the Dlx5−/− sample, Msx1 expression was comparable to that of wild type (FIGS. 5M,N,Q,R). A recent study failed to show that Msx1 and Dlx5 expression overlaps in the anterior palatal mesenchyme (Levi et al., 2006). Our data clearly demonstrate that Dlx5 is expressed in the anterior palatal mesenchyme (FIG. 1C) and was unaffected in the Msx1−/− sample (FIGS. 5O,P,S,T). Thus, Msx1 and Dlx5 do not appear to regulate each other's expression in the palate.

Rescue of cleft palate via modulation of Shh signaling. To investigate the stimulation of palatal mesenchymal cell proliferation by Shh signaling during palatogenesis, we treated E13.5 wild type palatal explants with either BSA- or Shh-beads. Palatal mesenchyme cell proliferation was enhanced following Shh treatment (FIGS. 6A,B inserts, C). Moreover, ectopic Shh did not affect apoptosis in the MEE or palatal fusion (FIG. 6A-E). Explants treated with BSA or Shh beads were both able to fuse following two days of culture. Thus, increased Shh signaling has a stimulatory role on cell proliferation in the palatal mesenchyme. Furthermore, overexpressing Shh does not appear to have a major Impact on the MEE cells.

To further support a role for expanded Shh signaling in the absence of Dlx5 is responsible for the increased palatal mesenchymal cell proliferation and the rescued palatal fusion in Msx1−/−; Dlx5−/− mice, we blocked Shh signaling with anti-Shh antibodies. In wild type, Dlx5−/−, and Msx1−/−; Dlx5−/− palatal explants, anti-Shh antibody treatment resulted in reduced mesenchymal cell proliferation activity compared to the BSA treated explants (FIG. 6F-L). Treatment with anti-Shh antibody blocked the rescued cell proliferation in the palatal mesenchyme of Msx1−/−; Dlx5−/− mice (FIGS. 6H,K,L). Our data is consistent with a role for increased Shh signaling being responsible for the rescue of cell proliferation in the Msx1−/−; Dlx5−/− mutant palate.

To confirm the restored mesenchymal proliferation is a result of loss of Fgf7 expression in the nasal side of palatal shelf, we blocked Fgf7 signaling with neutralizing antibody. In both wild type and Msx1−/− palatal explants, anti-Fgf7 antibody treatment resulted in increased mesenchymal proliferation activity compared to mouse IgG1 treated explants (FIG. 6-Q). Furthermore, in Msx1−/− palatal explants treated with anti-Fgf7, the mesenchymal proliferation rate was restored to a level comparable to that of wild type controls without anti-Fgf7 treatment (FIG. 6Q).

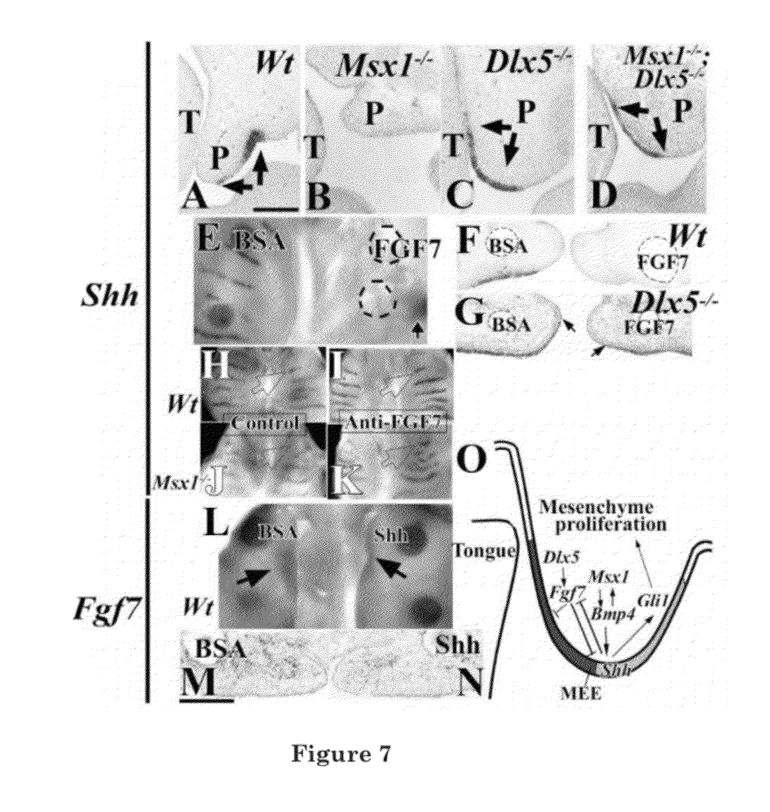

Msx1 and Dlx5 antagonistically regulate Shh expression to control cell proliferation in the palatal mesenchyme. The requirement for Shh signaling in the genetic rescue of cleft palate in Msx1−/−; Dlx5−/− mice suggests that Shh may be regulated by both Msx1 and Dlx5. This hypothesis predicts that Shh expression should be altered as a result of null mutation of either Msx1 or Dlx5. In wild type palatal shelves at E13.5, Shh expression was restricted to the oral side of the palatal epithelium and limited to defined stripes that correspond to the developing rugae (FIG. 7A and see FIGS. 2I, 4B). In the epithelial cells covering the palatal shelf of Msx1−/− mice, Shh expression was not detectable (FIG. 7B and see FIG. 2O). Loss of Dlx5 resulted in expanded Shh expression in the palatal epithelium (FIG. 7C and see FIG. 2J). This medial expansion of Shh signaling suggests that Dlx5 was required to suppress Shh expression in the palate. We also detected a similar expansion of Shh expression in the palatal epithelium of Msx1−/−; Dlx5−/− mutant mice (FIG. 7D). Thus, expanded Shh expression is independent of Msx1 signaling in the Dlx5 mutant. Gli1 is a mediator and target for the Shh pathway that enables us to detect Shh-responsive cells. We found that Gli1 was expressed in the oral half of wild type palatal mesenchyme adjacent to the epithelium where Shh was expressed (see FIG. 1G-I and FIG. 3A). In Dlx5−/− palate, Gli1 expression expanded into the nasal half of the palatal shelf (see FIG. 3B) corresponding to the expansion in Shh expression (FIG. 7C). In Msx1−/− palate, however, Gli1 expression remained restricted to the oral half of the palate (FIG. 3C). Therefore, we conclude that Msx1 and Dlx5 antagonistically regulate Shh expression in the palatal epithelium, however the capacity of palatal mesenchyme to respond to Shh signaling is independent of Msx1 status.

The expansion of Shh expression into the nasal side of the palatal epithelium in Dlx5−/− mice suggests that Dlx5 is required for restricting Shh expression to the oral side of the palatal epithelium. However, Shh is expressed in the oral side of the palatal epithelium (see FIG. 1G), whereas Dlx5 is expressed in the nasal side of the palatal mesenchyme (see FIG. 1C). This spatial relationship and the fact that Dlx5 functions as a transcription factor suggest that a Dlx5 downstream target gene in the palatal mesenchyme mediates the restriction of Shh expression. Endogenous Fgf7 is expressed in the nasal half of the palatal mesenchyme (Dlx5 expressing domain), whereas Shh is absent from the corresponding palatal epithelium (see FIGS. 1C,D,G). In the Dlx5 mutant, Fgf7 expression is specifically diminished in the nasal half of the palatal mesenchyme but Fgf7 expression in the craniobase persists (FIG. 3H), suggesting that Dlx5 is required for Fgf7 expression in the nasal half of the palatal mesenchyme. Fgf7 has previously been shown to inhibit Shh signaling during lung and limb development (Bellusci et al., 1997; Yonei-Tamura et al., 1999). To test if Fgf7 can inhibit Shh expression in the palatal epithelium, we placed Fgf7 beads into E13.5 palatal shelves in vitro and analyzed Shh expression. After one day of culture in serumless, chemically-defined media, Fgf7 bead treated samples showed a dramatic reduction of Shh expression in the palatal epithelium compared to BSA treated samples (FIGS. 7E,F). Furthermore, Fgf7 beads were able to inhibit Shh expression in the palatal epithelium of Dlx5−/− samples (FIG. 7G), suggesting that Dlx5-dependent Fgf7 expression is sufficient to inhibit the expression of Shh in the nasal half of palatal epithelium during palatogenesis. To confirm the inhibitory effect of Fgf7 on Shh expression, we then treated wild type and Msx1−/− palatal explants with anti-Fgf7 neutralizing antibody. Inhibition of Fgf7 signaling enhanced Shh expression in both wild type and Msx1 mutant mice (FIGS. 7I,K) and restored Shh expression in anterior region of the palatal shelf (FIG. 7K). Interestingly, exogenous Shh repressed Fgf7 expression (FIG. 7L-N), suggesting a feedback loop in Fgf7/Shh signaling interaction in regulating palatogenesis.

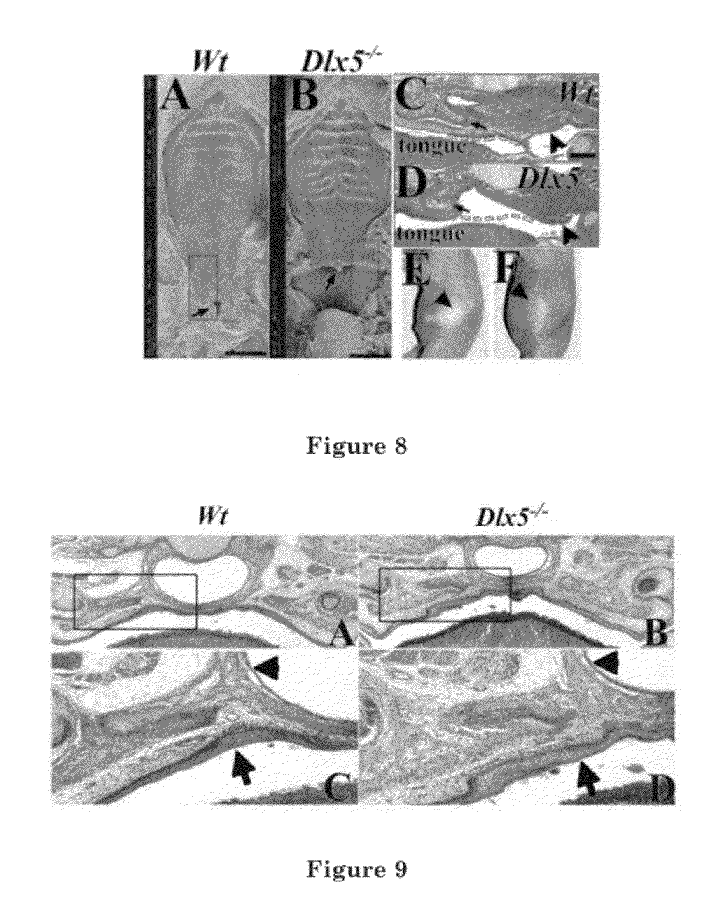

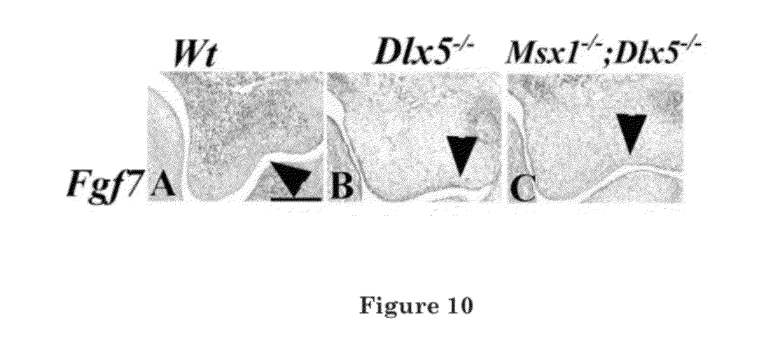

Dlx5 is critical for patterning the soft palate. In the posterior part of the palate, we discovered additional defects with complete phenotype penetrance in Dlx5−/− mice. These included a shortened, detached soft palate and the presence of a uvula-like structure (FIG. 8B). Wild type mice have a soft palate with a posterior border attached to the pharyngeal wall (FIGS. 8A,C). The shortened soft palate in Dlx5−/− mice resembles velopharyngeal insufficiency and fails to provide an adequate seal between the nasal and oral pharynx (FIGS. 8B,D). This anatomical defect also appears to cause air to enter the gastrointestinal tract, as the stomachs of all Dlx5−/− newborns were air-inflated (FIGS. 8E,F).

### Discussion

Oral-nasal patterning of the palatal shelf. In this study, we report the existence of oral-nasal patterning and its molecular regulation in the developing palatal shelf. Specifically, phospho-Smad1/5/8 antibody staining marks the activation of BMP signaling and appears to be restricted to the nasal side of the palatal mesenchyme. Interestingly, the expression of Bmp4 and Bmp2 is uniform throughout the palatal mesenchyme (Zhang et al., 2002). The expression of other members of the BMP and GDF families has not been thoroughly investigated. The functional significance of Bmpr1 has been demonstrated in a recent study, in which conditional inactivation of Bmpr1a in the CNC-derived mesenchyme results in cleft lip and palate (Liu et al., 2005). However, the distribution of Bmpr1a in the palatal mesenchyme still needs to be investigated in order to provide an explanation for the asymmetrical activation of BMP signaling in the palatal mesenchyme. It remains possible that the regional differential expression of Bmp receptor controls the establishment of the BMP-responsive domain in the palatal mesenchyme. Alternatively, it is plausible that there is an inhibition of BMP signaling activation on the oral side of the palatal mesenchyme. Further analysis will elucidate the molecular mechanism that controls the asymmetrical activation of BMP signaling in the palate that ultimately results in the formation of palatal bone towards the nasal side of the palate. Significantly, our study demonstrates that, although the palatal mesenchyme is populated with CNCderived cells, they are not a homogenous population and need to be evaluated and distinguished by molecular marker analysis. This insight changes our approach in the evaluation of the CNC-derived palatal mesenchyme during palatogenesis.

The restricted expression of Dlx5 on the nasal side and the expansion of the oral side growth of Dlx5−/− palate clearly suggest that Dlx5 actively participates in the oral-nasal patterning of the developing palate. In contrast, loss of Msx1 does not alter the oral-nasal patterning of the palate. Thus, we conclude that Msx1 is not involved in oral-nasal patterning of the palate.

Fgf7 is expressed in the nasal region of the palatal mesenchyme and exogenous Fgf7 beads inhibit the expression of Shh in the nasal region of the palatal epithelium, strongly suggesting that Fgf7 is a member of the hierarchy that determines oral-nasal patterning of the palatal mesenchyme. The null mutation of Fgf7 in mice did not generate an obvious palatal phenotype (Guo et al., 1996), as is also the case for the majority of Dlx5−/− mice (Depew et al., 1999).

The restricted expression of Shh in the oral side of the palatal epithelium and of Gli1 and Fgf10 in the oral side of the palatal mesenchyme suggests that Shh signaling is critical for the development of the oral side of the palate. In the developing palatal shelf, the cell surface receptors for Shh (Ptch1 and Smo), hedgehog signaling inhibitors (Hhip1 and Gas1), and Hh-signaling mediator (Gli-family zinc-finger transcription factors) are expressed in the palatal epithelium and mesenchyme (Rice et al., 2006). Conditional inactivation of Shh in the epithelium results in dramatic shortening of the palatal shelves and a wide cleft palate, whereas conditional inactivation of Smo in the epithelium does not disrupt palatogenesis (Billiard et al., 2005), consistent with the model that the underlying palatal mesenchyme is the recipient of Shh signals. Furthermore, mutations in Gli2 or Gli3 cause facial abnormalities including cleft palate (Mo et al., 1997). We conclude that it is likely that the restricted expression of Shh in the palate is the result of the patterning, in contrast to the setting in the spinal cord, where Shh functions as an inductive signal of patterning. Gli1, which marks the Shh-responding cells (Hooper and Scott, 2005; Lum and Beachy, 2004), is a stronger candidate for the molecule responsible for the patterning of the oral side palatal mesenchyme, because its expression is restricted to the oral half of the palate (see FIG. 3). Our results suggest that, although Shh is not involved in the oral-nasal patterning of the palatal shelf, Shh-responsiveness determines the oral half of the palate. The persistence of Gli1 expression in Msx1−/− palates indicates that the Gli1 expression in the oral side of the palatal mesenchyme does not require the presence of Shh and is not under the regulation of Msx1. Interestingly, a recent study shows that Gli1 and Ptch1 expression are down-regulated in the palatal mesenchyme following the ectoderm specific inactivation of Shh gene (Lan and Jiang, 2009). To date, studies have shown that hedgehog and Tgf-β signaling induces Gli1 expression whereas Snail/Slug, and Notch signaling inhibits Gli1 expression (Katoh and Katoh, 2009). Further analysis is necessary to reveal the comprehensive regulation of Gli1 expression during palatogenesis.

The function of Shh signaling in palatal fusion. Previous studies have shown that Shh functions as a critical mitogenic factor for the palate mesenchyme through its coordination of Fgf10-Fgfr2b and Msx1-Bmp4 signaling networks (Rice et al., 2004; Zhang et al., 2002). We have shown here that the actively proliferating palatal mesenchymal cells are those adjacent to the SM-expressing epithelial cells. This spatial correlation is consistent with the restricted range of Shh activity in the limb bud (Li et al., 2006). Shh beads are able to stimulate mesenchymal proliferation in palate explants in vitro, and mesenchymal cell proliferation is reduced in palate explants treated with anti-Shh antibody. In Dlx5−/− palates, expanded Shh signaling and increased cell proliferation in the palatal mesenchyme might contribute to the overgrowth of rugae and eventual folding of the palatal shelf. On the other hand, compromised Shh expression is accompanied by a reduction in cell proliferation in both palatal mesenchyme and epithelium in Fgf10−/− mice (Rice et al., 2004). Previous studies have shown that the Shh signaling cascade is regulated at multiple levels. Cholesterol modification can restrict the spread of Shh and control the range and shape of the Shh morphogen gradient (Li et al., 2006). At the intracellular level, the combined activities of hedgehog signaling inhibitors (Hhip and patched 1) are crucial for tightly controlled Shh activity during pancreas development (Kawahira et al., 2003) and during the initiation of tooth development (Cobourne et al. 2004). Another important mechanism affecting Shh signaling is tissue-tissue interaction. Previous study shows that Msx1 controls the expression of Bmp4, which in turn positively regulates Shh expression during palatogenesis (Zhang et al., 2002). Fgf10 positively regulates Shh expression through Fgfr2b in the palatal epithelium (Rice et al., 2004). We have shown in this study that transcriptional antagonism between Msx1 and Dlx5 in regulating Shh expression ensures the precise spatial-temporal control of Shh signaling during palatal shelf development. More specifically, Msx1-mediated BMP4 signaling is responsible for inducing Shh signaling in the palatal epithelium, whereas Dlx5 is responsible for the indirect inhibition of Shh signaling in the nasal side of the palatal epithelium. As the result of this antagonistic control, there is an asymmetrical distribution of Shh signaling in the palatal epithelium. This specific Shh expression pattern is critical for the growth and elevation of palatal shelf prior to fusion.

Shh as a potential target for repairing a specific group of cleft palate cases We propose that Shh signaling is a potential target for the repair of the group of cleft palate cases that result from the failure of palatal shelves to meet at the midline due to compromised palatal mesenchymal proliferation. Restored Shh expression in the palatal epithelium of Msx1−/−; Dlx5 −/− mice is sufficient to trigger the Shh signaling cascade leading to palatal mesenchyme proliferation and fusion, supported by the evidence that inhibition of Shh signaling in Msx1−/−; Dlx5−/− mice reverses the rescue of palatal mesenchyme proliferation. Furthermore, Shh is the converging point for Bmp signaling and Fgf signaling during the expansion stage of palatogenesis (Rice et al., 2004; Zhang et al., 2002). Taken together, we conclude that modulating Shh signaling may provide an opportunity to direct palatal shelf growth and rescue palatal fusion in mutant models with insufficient palatal shelf growth and cleft palate defect.

Dlx5 and soft palate development The discovery of a soft palate defect in Dlx5−/− mice may have significant implications from an evolutionary perspective. Unlike humans, mice have a soft palate that is attached to the posterior pharyngeal wall. The epiglottis is above the level of soft palate; mice can therefore suckle and breathe at the same time. In humans, the posterior border of the soft palate is free and the epiglottis is below the posterior boarder of soft palate. This anatomical feature is an important advancement in human evolution because proper function of the soft palate is critical for speech development. Furthermore, soft palate, along with epiglottis and soft tissue structures within the larynx, acts as a valve to prevent food and liquid from entering lower parts of the respiratory tract. We have found that loss of Dlx5 results in a shortened soft palate in mice. Because Dlx genes are known for their function in regulating the identity of craniofacial structures and morphological novelty in the vertebrate lineage (Beverdam et al., 2002; Depew et al., 2002; Neidert et al., 2001), our data suggest that Dlx5 plays an important role in patterning the proximal region of the pharyngeal arch derivatives.

Interaction of Msx and Dlx signaling Our data clearly demonstrate that Msx1 and Dlx5 operate in parallel in regulating downstream target gene expression during palatogenesis. Recent studies show that the function of Msx genes is to control CNC cell cycle progression as loss of both Msx1 and Msx2 genes results in defects in CNC cell proliferation and survival, but does not affect the expression of Dlx5 and the patterning of the branchial arch (Han et al., 2003; Ishii et al., 2005). On the other hand, members of the Dlx gene family mainly control the patterning of the craniofacial skeleton as loss of both Dlx5 and Dlx6 genes results in homeotic transformation of the lower jaw to upper jaw (Depew et al., 2002). Interestingly, the expression of Msx1 and Msx2 is reduced in the first branchial arch of the Dlx5; Dlx6 compound mutant samples, suggesting the Msx genes may function downstream of Dlx5 and Dlx6 in regulating the patterning of the branchial arch derivatives (Depew et al., 2002). In tooth development, however, Msx1 is required for Dlx5 expression in the dental mesenchyme. Msx1 and Dlx5 appear to work synergistically to regulate tooth and alveolar bone development (Zhang et al., 2003). Clearly, Msx1 and Dlx5 can work either antagonistically or synergistically to regulate downstream target gene expression, depending on the context. The outcome of this interaction and whether Msx1 and Dlx5 work in parallel or in sequential manner depends on the cell and tissue type where the interaction takes place. This operating logic allows for diverse outcomes associated with Msx1/Dlx5 interaction in regulating organogenesis. Equally important, perturbation of the Msx1/Dlx5 interaction may affect an array of downstream target genes and sets the stage for dysmorphogenesis.

### Example 2

### Composition and Method for Treating Craniofacial Malformation Based on a Novel TGF-β Signaling Mechanism

To investigate the mechanism of altered TGF-β signaling, we analyzed an established mouse model of cleft palate caused by neural crest specific inactivation of Tgfbr2 (Tgfbr2^{fl/fl}; Wnt1-Cre). We performed global gene expression analyses of the palatal tissue of Tgfbr2^{fl/fl}; Wnt1-Cre mutant and Tgfbr2^{fl/fl} control mice at embryonic day E14.5 (during palatal fusion, n=5 per genotype) to examine the downstream consequences of dysfunctional Tgf-β signaling during palate formation. In this comparison, we uncovered 291 probe sets representing transcripts that were differentially expressed [1.5-fold, <5% false discovery rate (FDR)], 148 more abundant in Tgfbr2^{fl/fl}; Wnt1-Cre (Table 1) and 143 more abundant in Tgfbr2^{fl/fl} control mice (Table 2). The genes identified as altered in Tgfbr2^{fl/fl}; Wnt1-Cre mice were consistent with cell proliferation defects, with significant reductions in the levels of transcripts related to cell cycle control, mitosis, and microtubule-based cell division, and with enrichment for transcripts related to the negative regulation of cellular proliferation (Table 2).

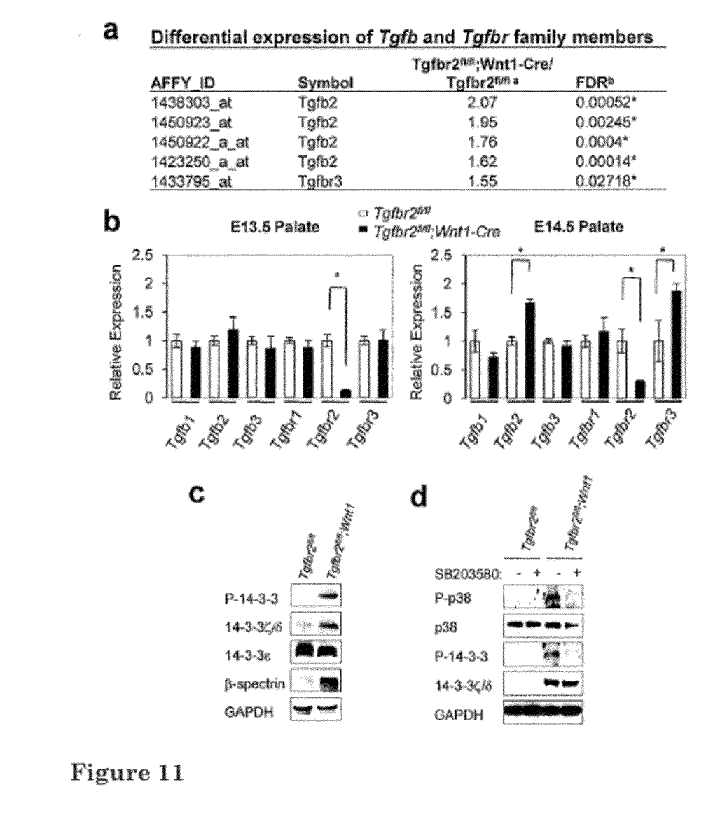

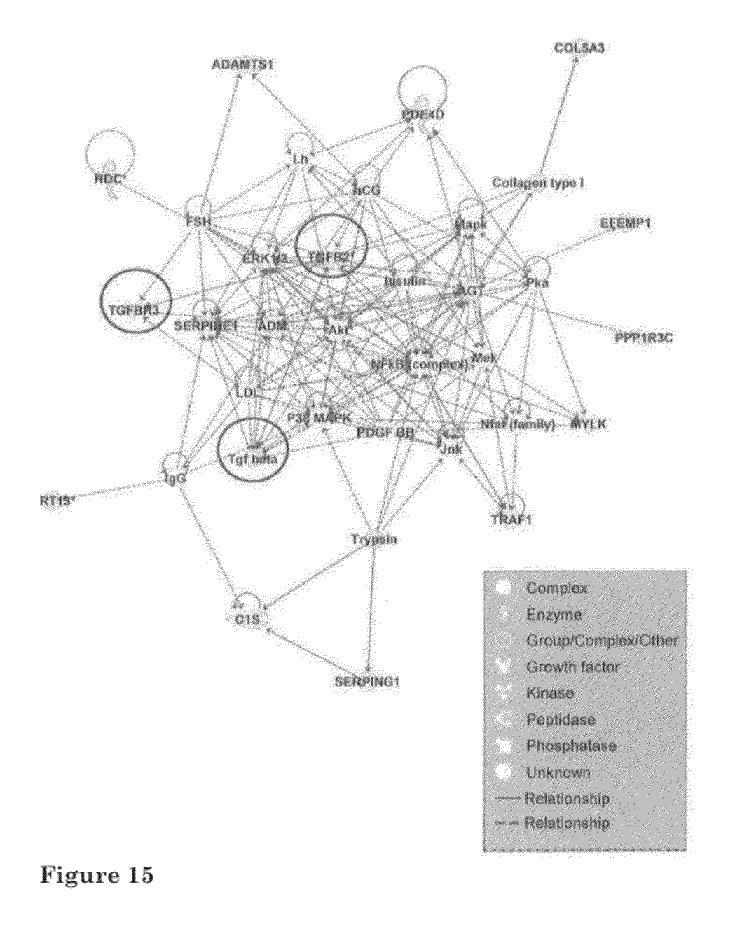

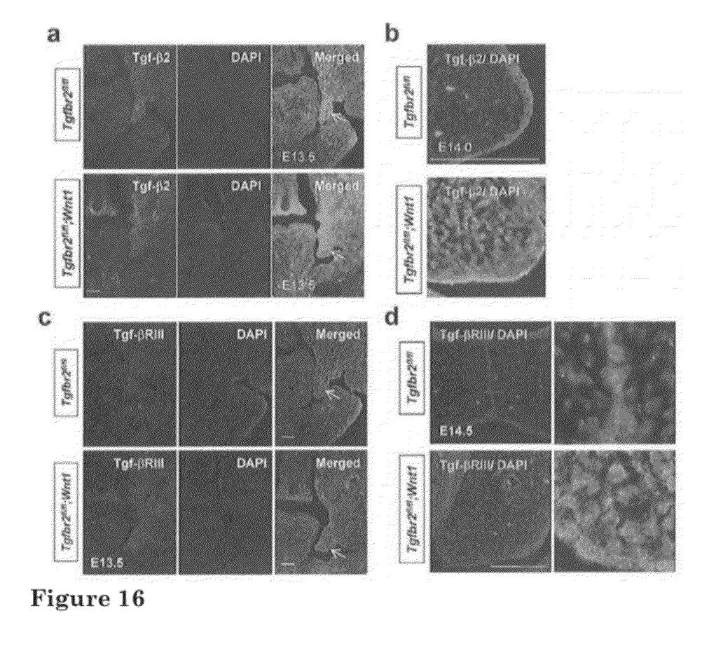

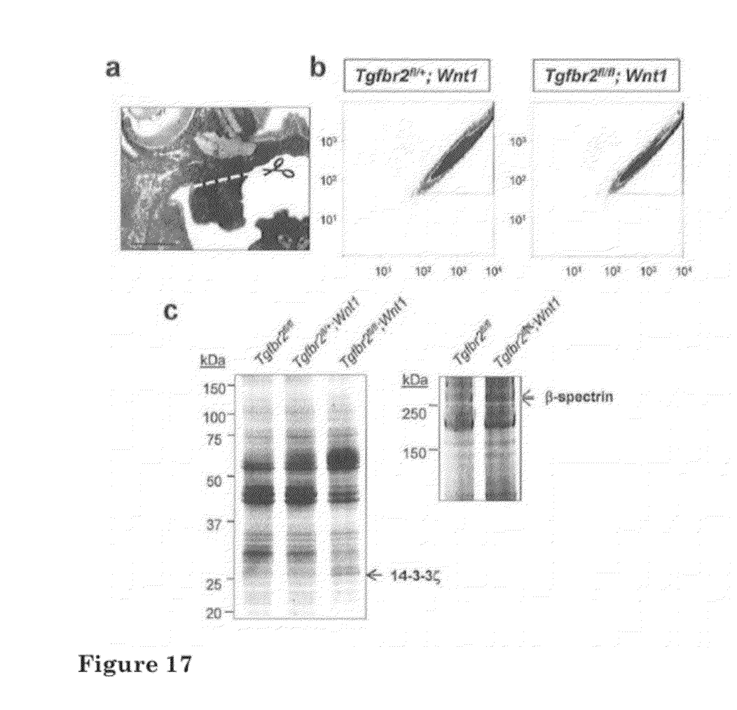

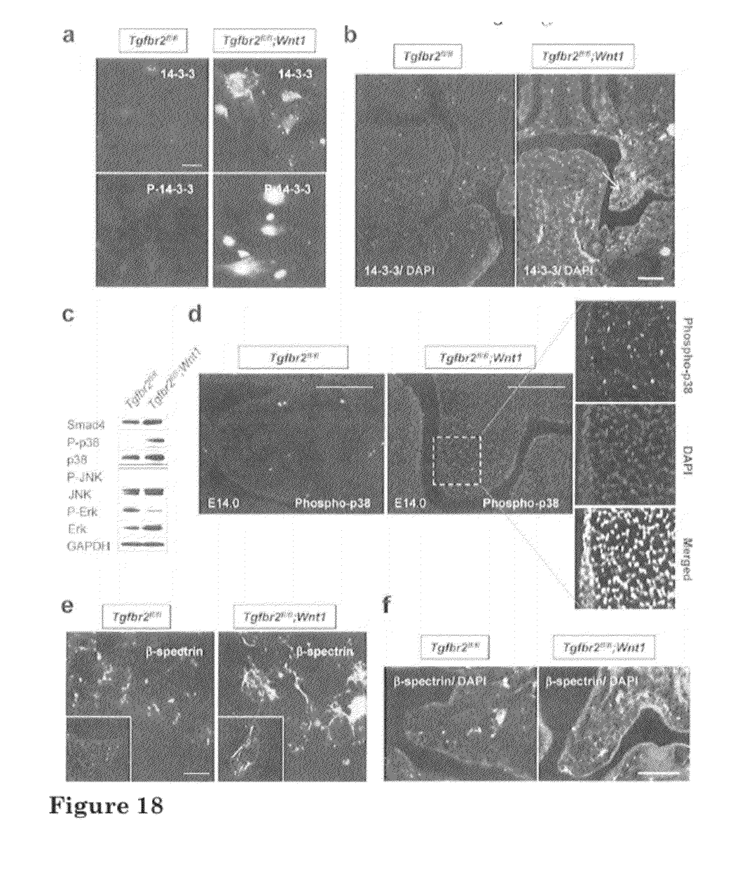

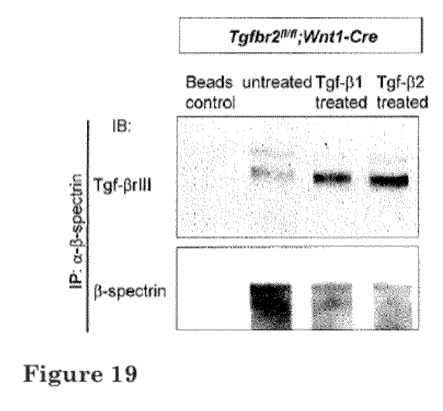

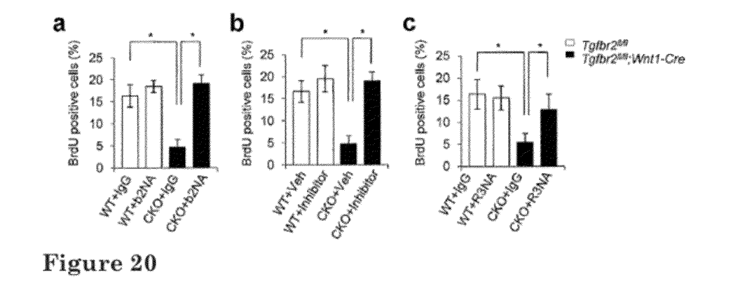

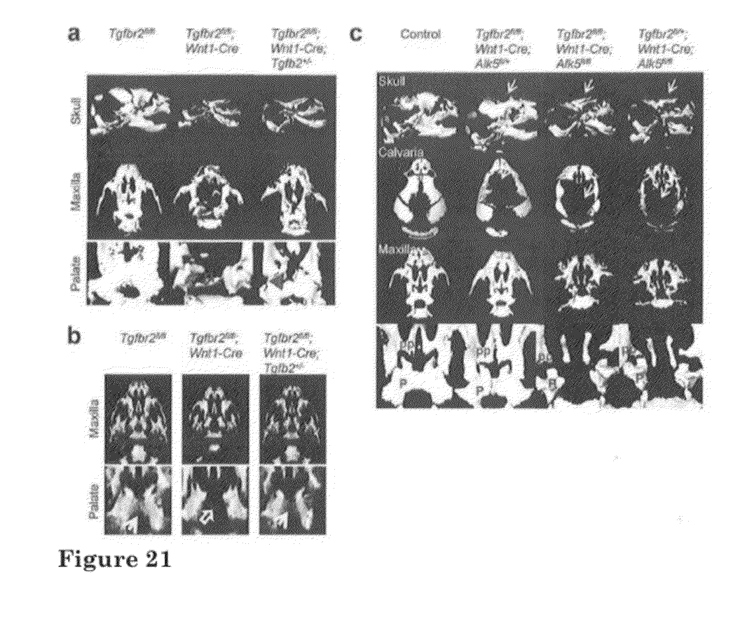

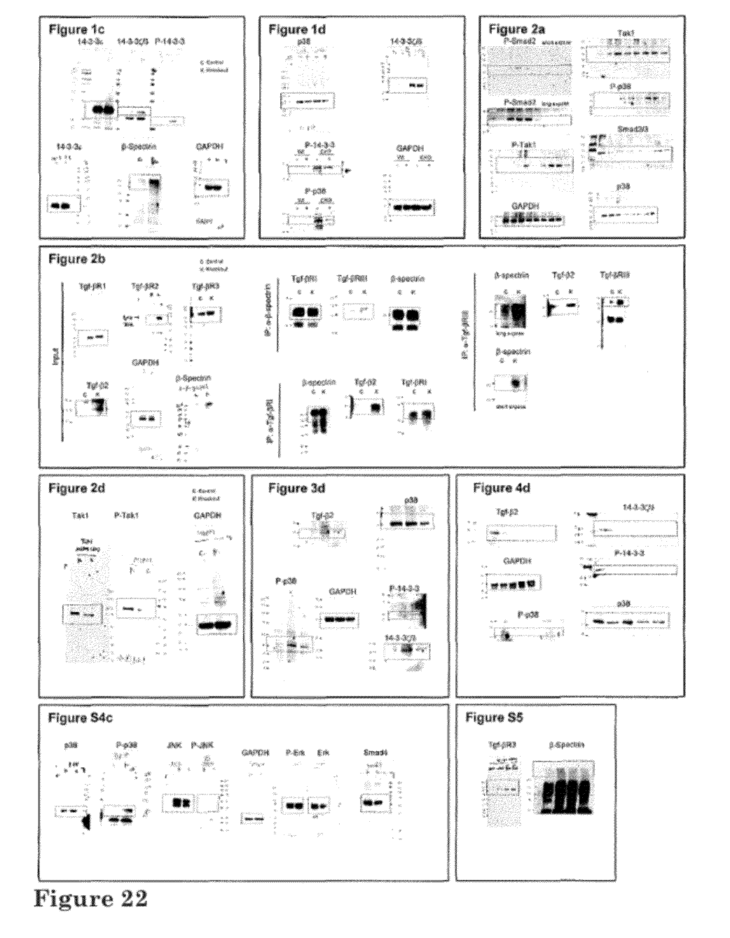

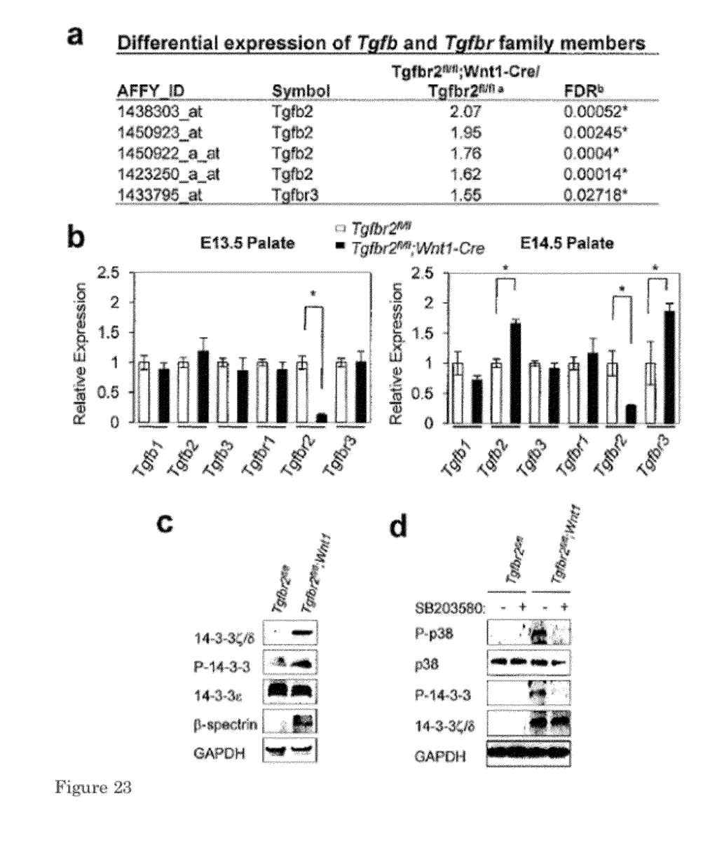

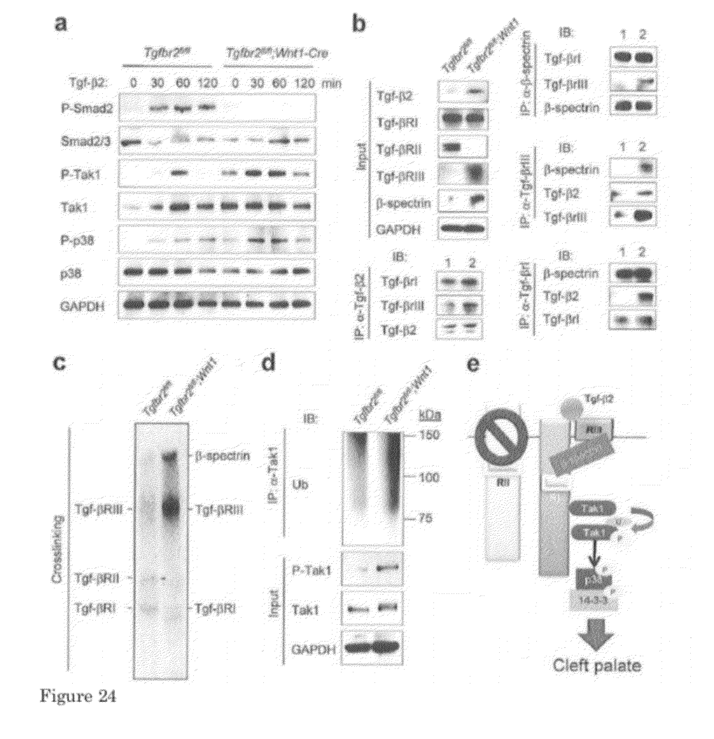

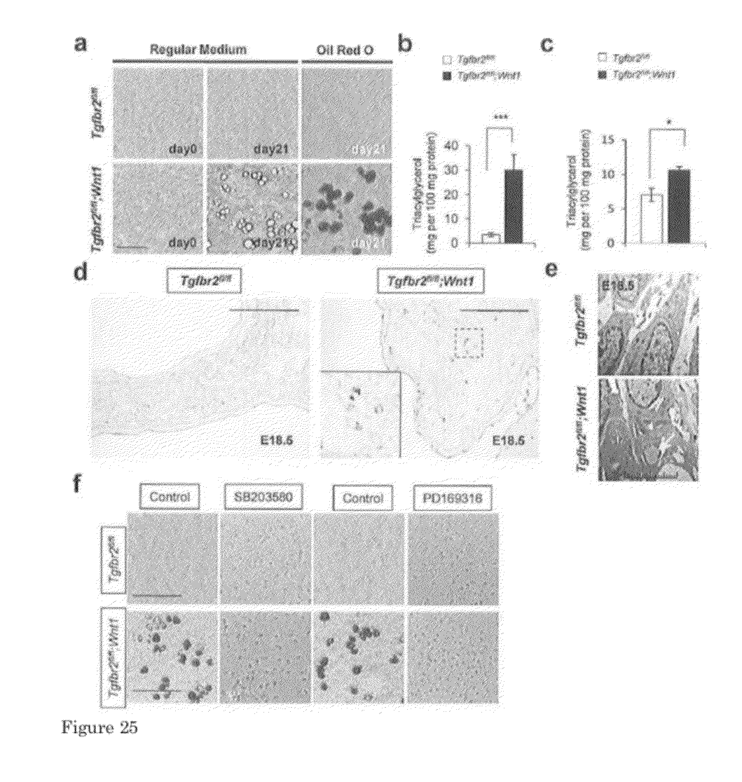

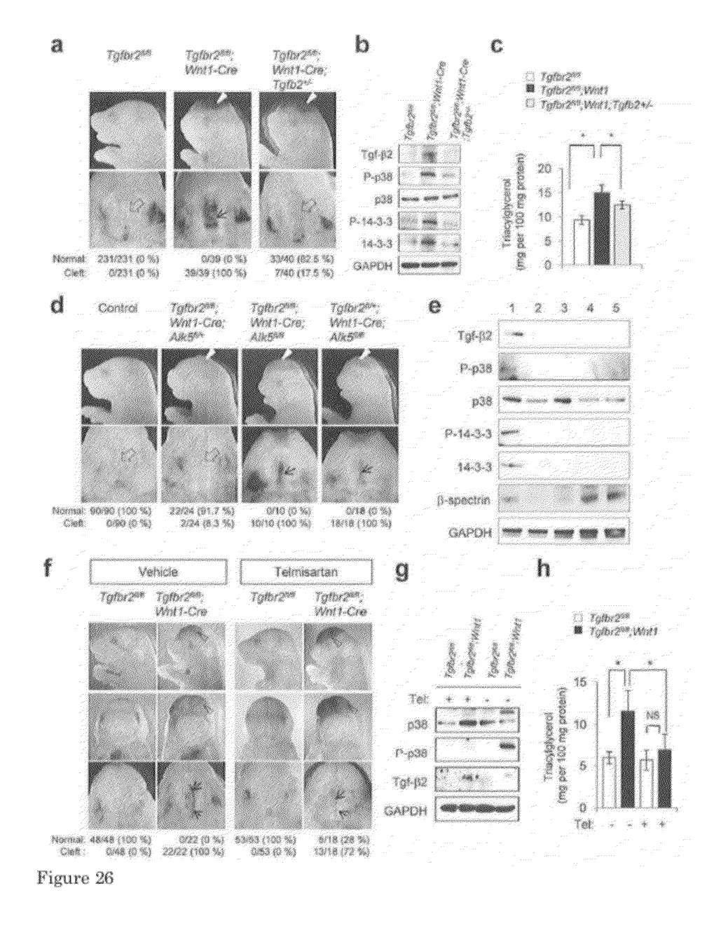

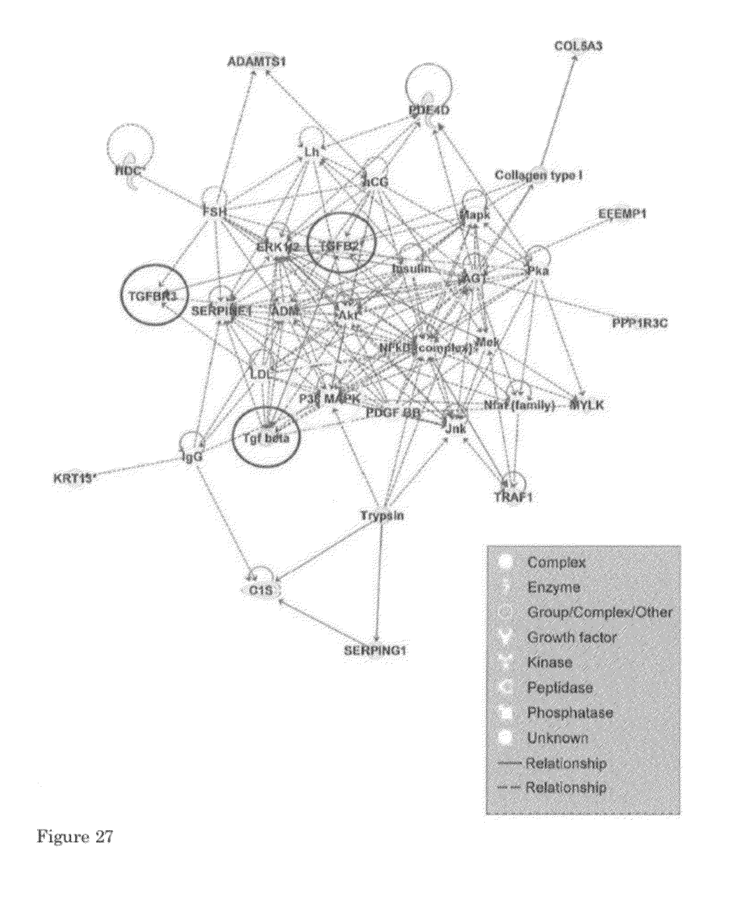

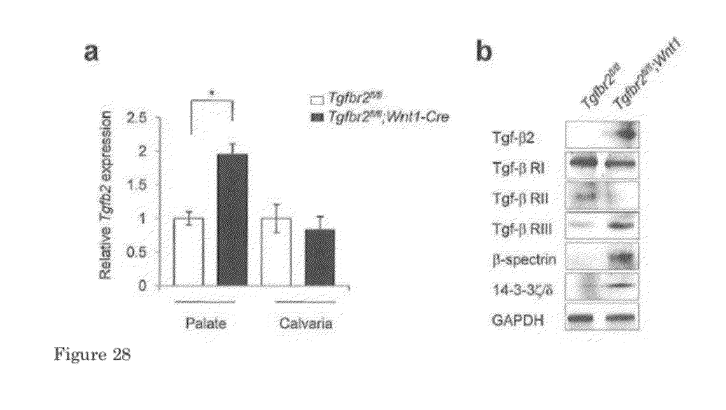

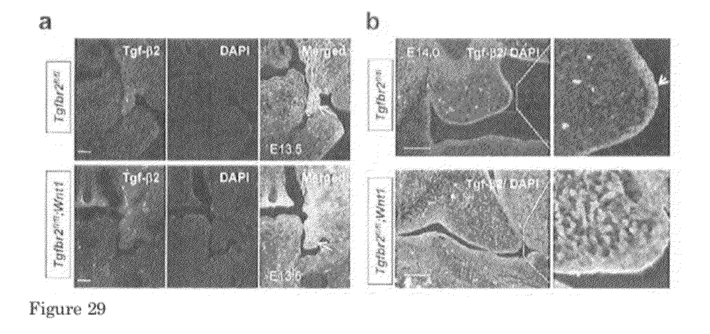

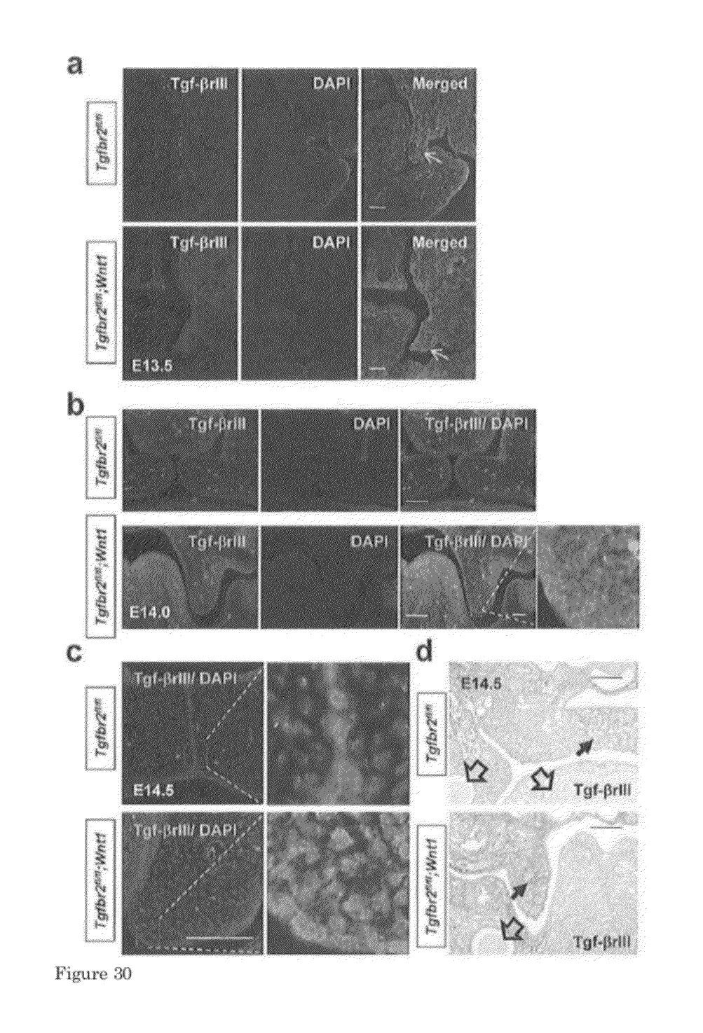

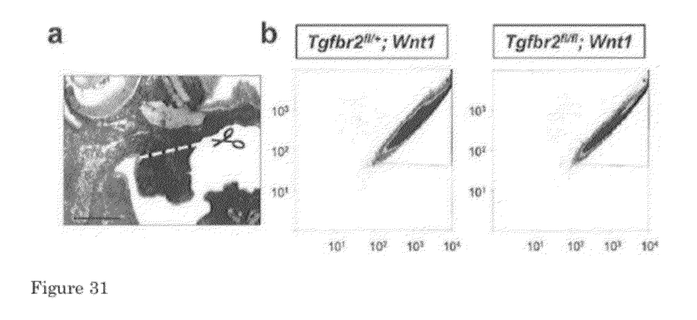

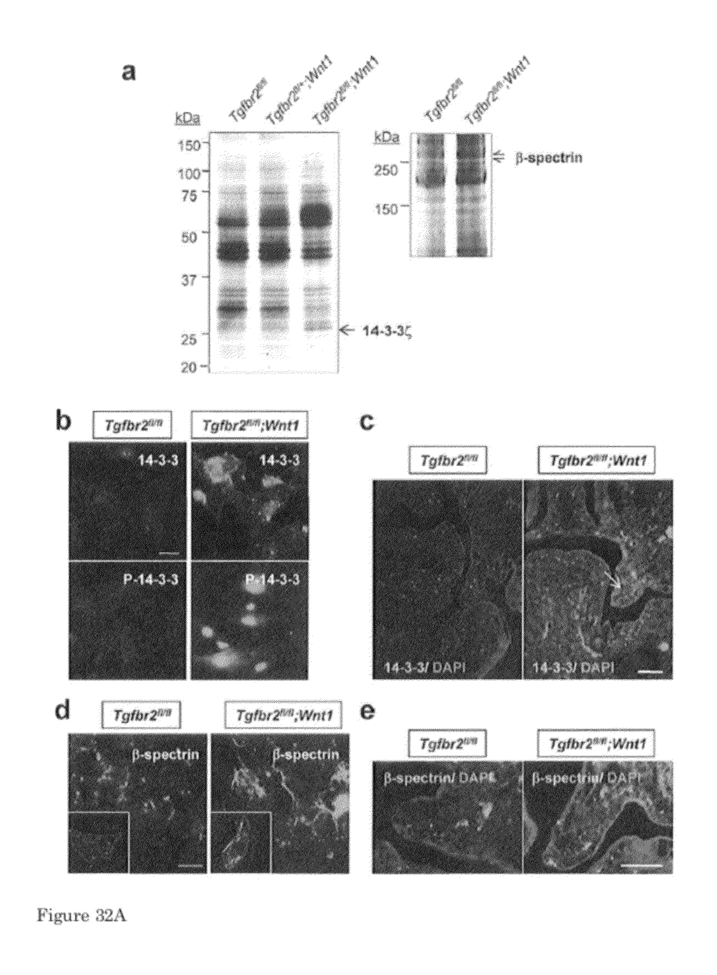

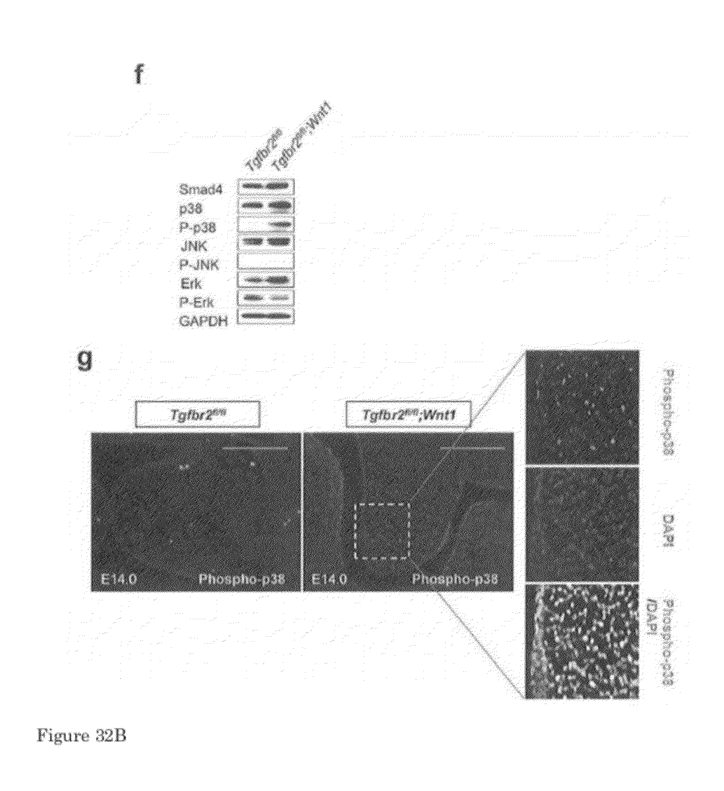

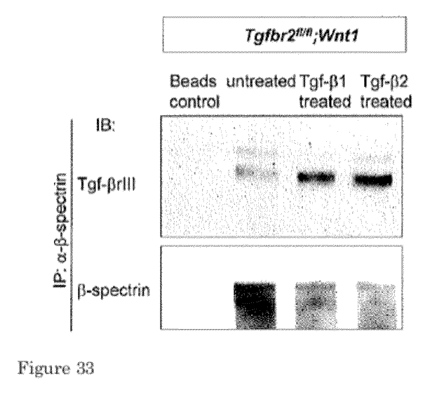

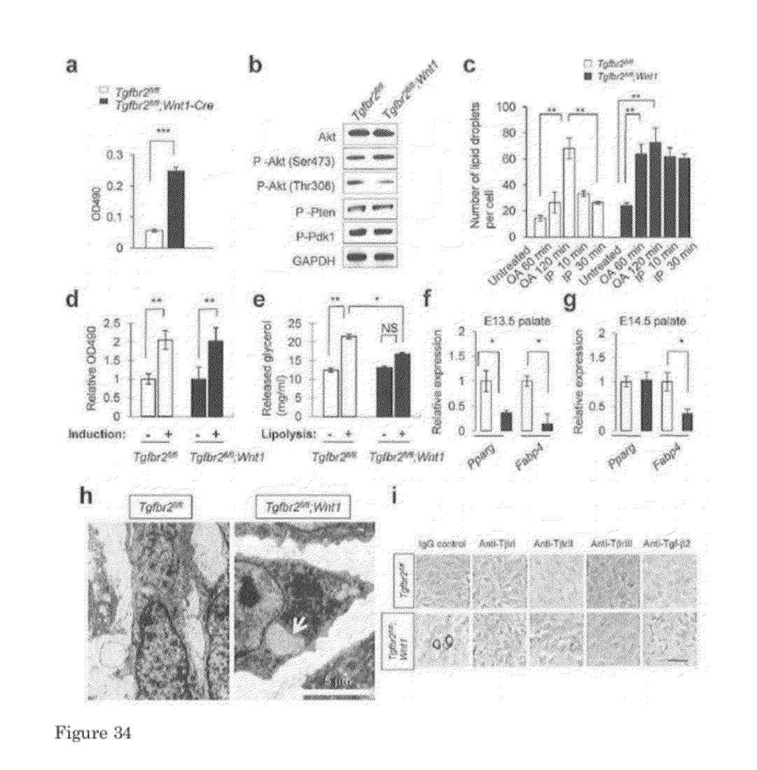

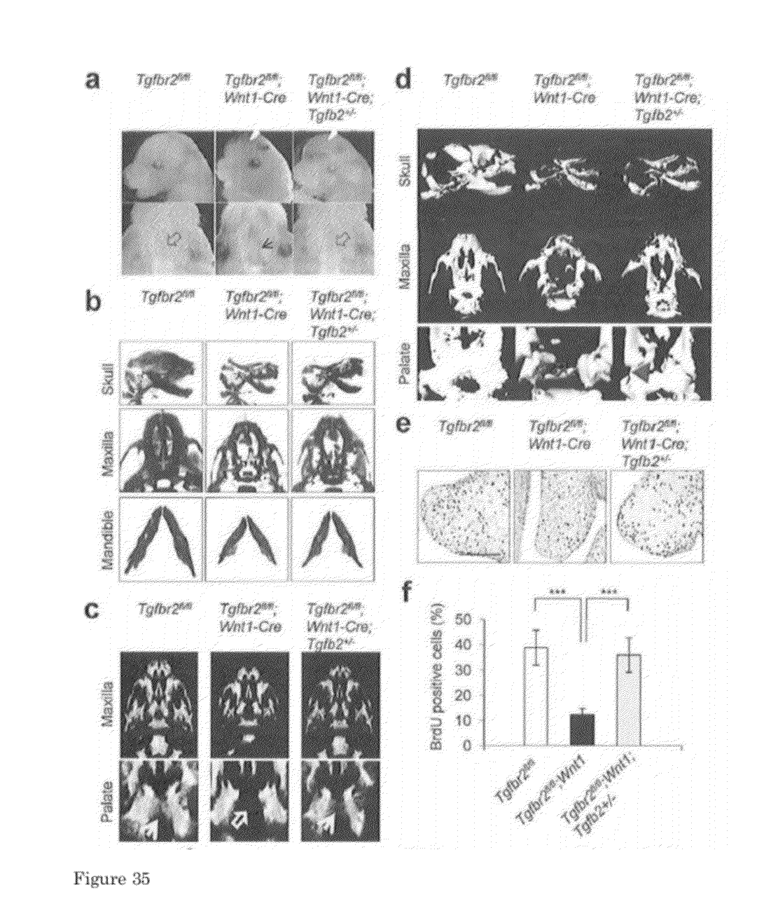

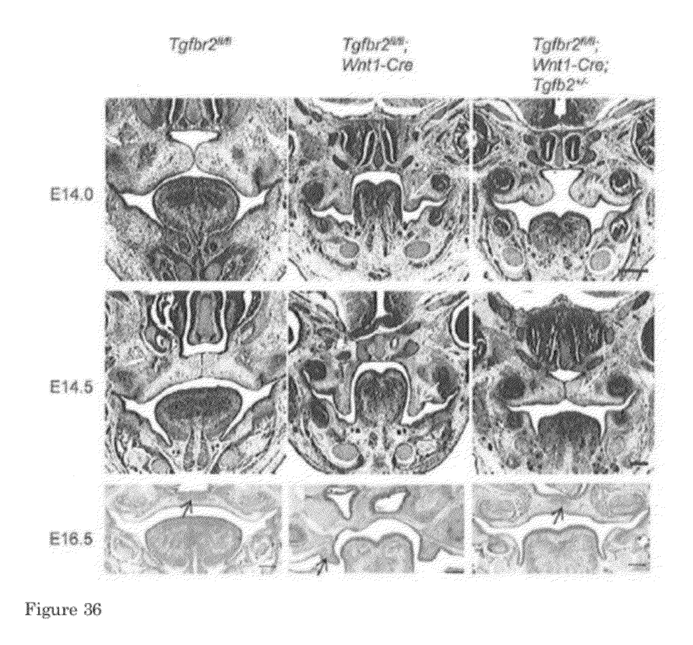

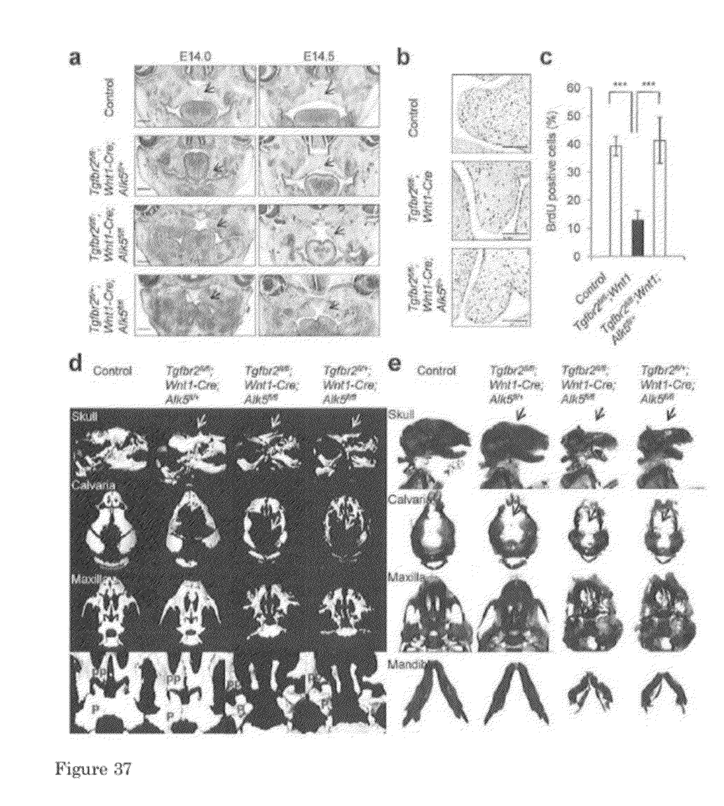

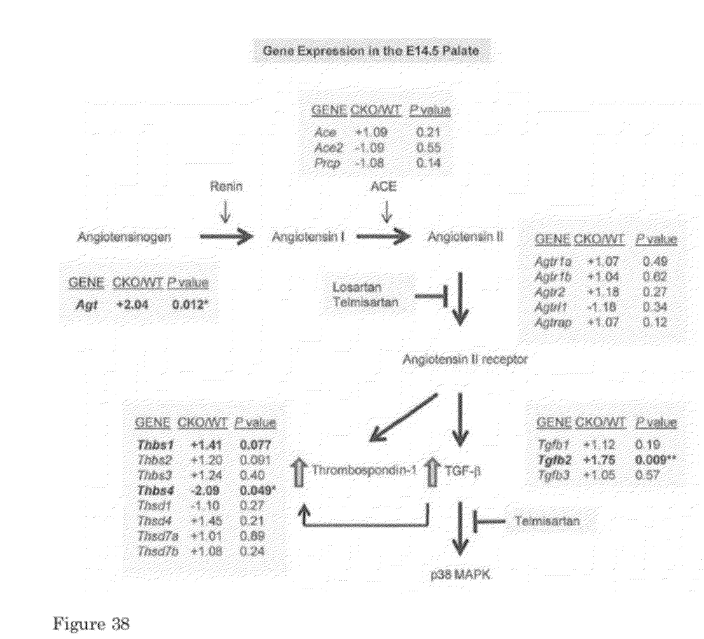

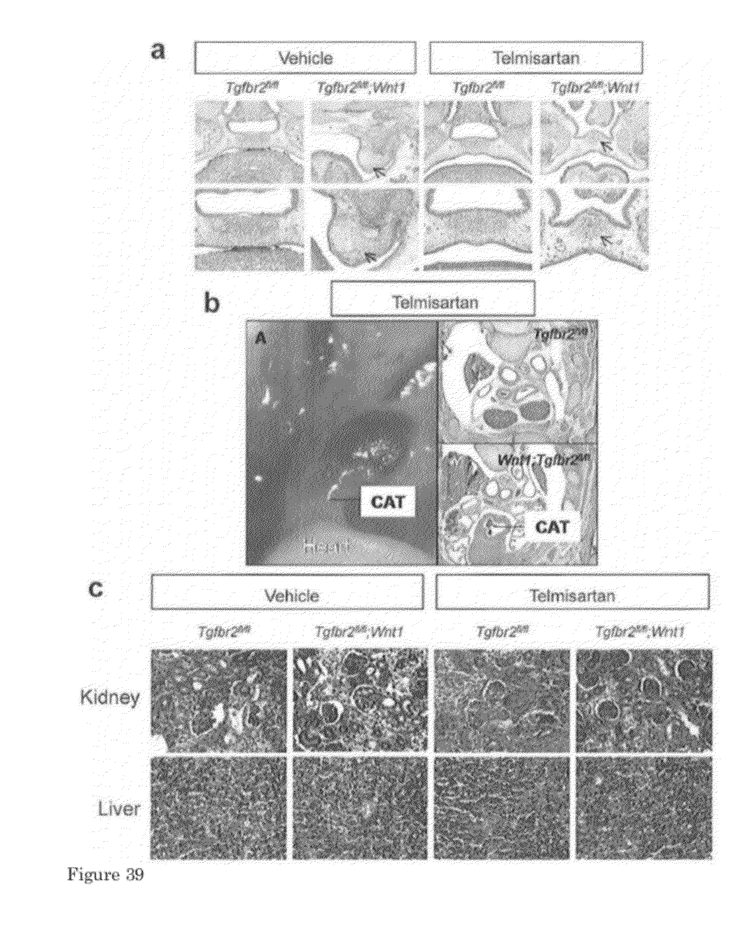

Next, we focused on the expression of genes related to Tgf-β signaling (FIG. 11a; FIG. 15). Strikingly, we discovered that both Tgfb2 and Tgfbr3 transcript levels were more abundant in the palate of the Tgfbr2^{fl/fl}; Wnt1-Cre mice than the control (FIGS. 11a, b). Tgf-β2 and Tgf-βRIII protein levels were also up-regulated in the palatal mesenchyme of Tgfbr2^{fl/fl}; Wnt1-Cre mice at E14.5 (FIG. 16). We propose that this represents a feedback loop whereby interrupted Tgfbr2-mediated signaling leads to an increased production of the Tgf-β ligand and signaling through an alternative Tgf-β receptor (i.e. Tgf-βRIII). Relevant to the latter observation, Tgf-β2 has a high affinity for Tgf-βRIII, a unique feature that distinguishes Tgf-β2 from other Tgf-β ligands.

To identify early changes in protein composition relevant to dysfunctional Tgf-β signaling, we performed an exploratory analysis of protein extracts from mouse embryonic palatal mesenchymal (MEPM) cells of E13.5 Tgfbr2^{fl/fl} (control) and Tgfbr2^{fl/fl}; Wnt1-Cre mice (FIG. 11c; FIG. 17, and Table 3). We found that 14-3-3ζ/δ and phosphorylated 14-3-3 were elevated in Tgfbr2^{fl/fl}; Wnt1-Cre MEPM cells compared with control cells (FIG. 11c; FIGS. 18a,b). 14-3-3 is a Tgf-βRI interacting protein that is phosphorylated by p38 mitogen-activated protein kinase (MAPK) and plays a key role in protein targeting and protein domain specific binding. Consistent with these results, p38 MAPK was activated only in Tgfbr2 mutant MEPM cells, but not in control cells (FIG. 11d; FIGS. 18c, d). Furthermore, phosphorylation of both p38 (aka Mapk14) and 14-3-3ζ/δ in Tgfbr2^{fl/fl}; Wnt1-Cre MEPM cells was inhibited by p38 MAPK inhibitor SB203580 (FIG. 11d).

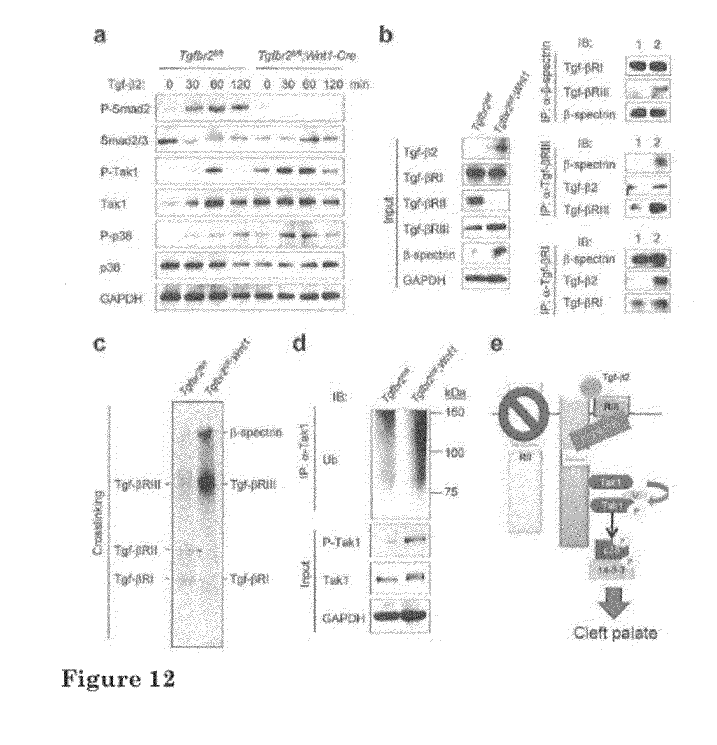

Similarly, β-spectrin, an adaptor protein of Tgf-βRI, was also up-regulated in Tgfbr2^{fl/fl}; Wnt1-Cre MEPM cells in both exploratory and confirmatory analysis (FIG. 11c; FIG. 17c and Table 3). β-spectrin expression was detectable in palatal mesenchymal cells of both Tgfbr2^{fl/fl}; Wnt1-Cre and control mice, but appears increased in Tgfbr2^{fl/fl}; Wnt1-Cre mice (FIGS. 18e, f). Consistent with its ability to interact with Tgf-β receptors, β-spectrin was mainly localized at the cell membrane in Tgfbr2^{fl/fl}; Wnt1-Cre mice (FIG. 18e). We also found that Smad-dependent Tgf-β signaling was compromised in Tgfbr2^{fl/fl}; Wnt1-Cre MEPM cells (FIG. 12a). Therefore, we propose that elevated Tgf-β2 levels in Tgfbr2 mutant cells initiate an intracellular signal in the absence of Tgfbr2, via a hitherto unidentified mechanism.

To analyze the mechanism of signaling initiated in the absence of Tgfbr2, we performed cross-linking and co-immunoprecipitation assays using anti-Tgf-β2, Tgf-βRI, Tgf-βRIII, and β-spectrin antibodies and extracts of control and Tgfbr2^{fl/fl}; Wnt1-Cre MEPM cells (FIGS. 12b, c). We found that Tgf-β2 binds more efficiently to Tgf-βRIII in the absence of Tgfbr2 (FIGS. 12b, c). Furthermore, β-spectrin bound more efficiently to the ligand-receptor complex and was co-immunoprecipitated with Tgf-βRIII and Tgf-βRI in Tgfbr2^{fl/fl}; Wnt1-Cre MEPM cells (FIGS. 12b, c; FIG. 19). These data indicate that a Tgf-β2/Tgf-βRIII/Tgf-βRI complex that includes β-spectrin forms in the absence of Tgfbr2 and likely transduces Tgf-β signaling in palatal mesenchymal cells.

Previous studies suggested that Tgf-βRIII was dispensable for Tgf-β-mediated signal transduction because it lacks an intracellular kinase domain and generally works as co-receptor of Tgf-βRI and Tgf-βRII to recruit Tgf-β ligands. In addition, recent findings indicate that Smad-independent MAPK pathways [p38 and c-Jun NH₂-terminal kinase (JNK)] are activated by Tgf-β receptor kinase-independent pathway via ubiquitination and phosphorylation of Tgf-β-activated kinase 1 (Tak1) after binding of Tgf-βligands. We hypothesized that the Tgf-β2/Tgf-βRIII/Tgf-βRI/β-spectrin complex might activate Smad-independent Tgf-β signaling using a Tak1 cascade. Indeed, ubiquitination and phosphorylation of Tak1 were up-regulated in Tgfbr2^{fl/fl}; Wnt1-Cre MEPM cells (FIG. 12d). Thus, our results show that, in the absence of Tgfbr2, elevated Tgf-β2 forms a complex with Tgf-βRIII/Tgf-βRI/β-spectrin and induces p38 MAPK signaling cascade in the palatal mesenchyme (FIG. 12e).

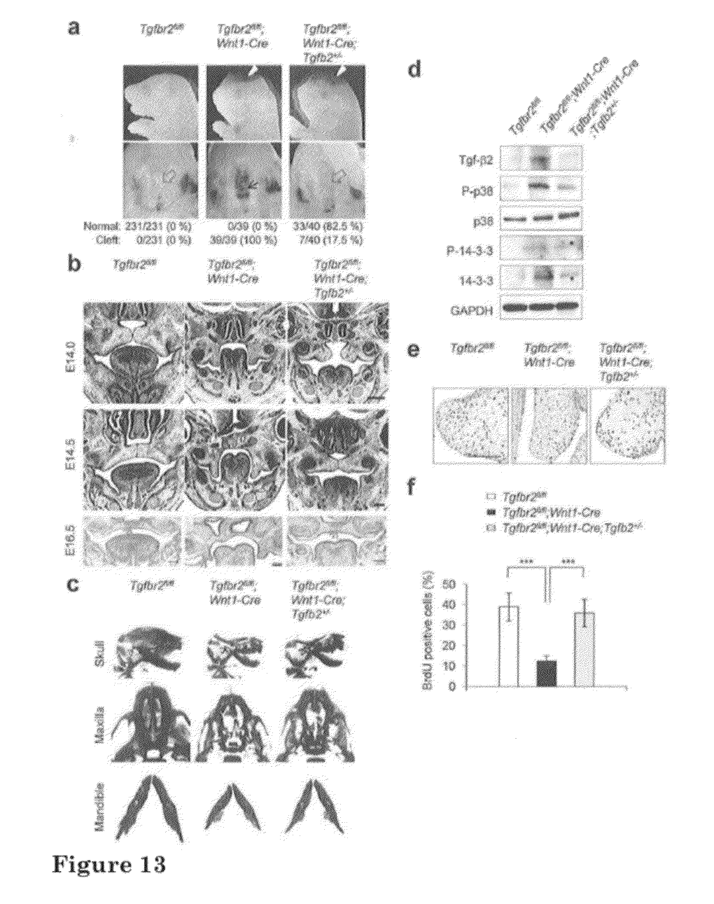

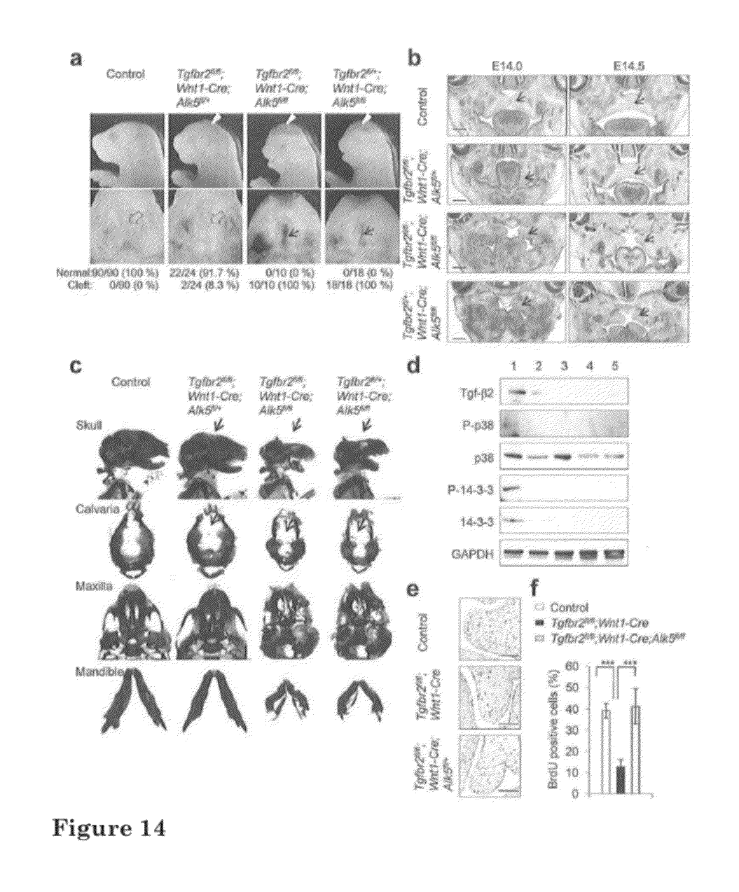

Our previous study showed that loss of Tgfbr2 results in a cleft palate with reduced CNCC proliferation in the palatal mesenchyme. We hypothesized that elevated Tgf-β2 may trigger a Smad-independent Tgf-β signaling cascade via a Tgf-βRI/RIII complex in the absence of Tgfbr2 and adversely affects CNCC proliferation during palate formation. Reduction of Tgf-β2 would thus increase CNCC proliferation and might prevent the cleft palate in Tgfbr2^{fl/fl}; Wnt1-Cre mice (FIG. 20a). To test this hypothesis, we generated Tgfbr2^{fl/fl}; Wnt1-Cre; Tgfb2^{+/-} mice. Indeed, Tgfbr2^{fl/fl}; Wnt1-Cre; Tgfb2^{+/-} mice formed palates normally with 82.5% phenotype penetrance (33/40) (FIG. 13a-c). Furthermore, the up-regulated p38 MAPK initially detected in Tgfbr2^{fl/fl}; Wnt1-Cre palates was restored to control levels in E14.5 Tgfbr2^{fl/fl}; Wnt1-Cre; Tgfb2^{+/-} palates (FIG. 13d). In addition, cell proliferation activity in the CNCC-derived palatal mesenchyme was restored to control level in Tgfbr2^{fl/fl}; Wnt1-Cre; Tgfb2^{+/-} palates (FIGS. 13e, f). In order to test the hypothesis that activation of ectopic Tgf-β signaling in Tgfbr2^{fl/fl}; Wnt1-Cre mice depends on the presence of Tgfbr1(Alk5) and Tgfbr3, we cultured palates with a neutralizing antibody for Tgf-βRIII or a p38 MAPK inhibitor SB203580 (FIGS. 16b, c). Cell proliferation defect was rescued by the treatment with both of the neutralizing antibody for Tgf-βRIII or the p38 MAPK inhibitor in ex vivo organ culture system. To test further our model, we generated Tgfbr2^{fl/fl}; Wnt1-Cre; Alk5^{fl/+} mice (FIG. 14). Significantly, a reduction of Tgfbr1/Alk5 restored cell proliferation and p38 MAPK activity, and rescued the cleft palate in Tgfbr2^{fl/fl}; Wnt1-Cre; Alk5^{fl/+} mice with 91.7% penetrance (22/24) (FIG. 14). Thus, our findings indicate that elevated Smad-independent p38 MAPK activation mediated through Tgf-βRI/RIII is responsible for the CNCC proliferation defect and failure of palatal fusion in Tgfbr2^{fl/fl}; Wnt1-Cre mice.

Although Tgfb2 haploinsufficiency rescued the CNCC proliferation defect and cleft palate in Tgfbr2^{fl/fl}; Wnt1-Cre mice, calvarial and mandibular defects were not rescued in Tgfbr2^{fl/fl}; Wnt1-Cre; Tgfb2+/− mice (FIG. 13c; FIGS. 21a, b). In contrast, Tgfbr1/Alk5 haploinsufficiency completely rescued the CNCC proliferation defect as well as palatal fusion, and partially rescued calvarial, maxillary and mandibular defects in Tgfbr2^{fl/fl}; Wnt1-Cre mice (FIG. 14c; FIG. 21c). One possible explanation is that, in addition to an elevated Tgfb2 expression, other Tgf-β ligands can still signal through Tgf-βIR/IIIR complex to cause adverse effects on craniofacial development. Therefore, a reduction of Tgf-β2 signaling alone does not prevent these developmental defects, but a reduction of Tgfbr1/Alk5 does. This reasoning is supported by the fact that almost all craniofacial defects are partially rescued in Tgfbr2^{fl/fl}; Wnt1-Cre; Alk5^{fl/+} mice. Alternatively, CNCC-derived palatal mesenchyme could be more sensitive to the level of Tgf-β2 signaling, which has been shown previously to induce differential developmental outcomes dependent on its expression level. For example, progenitor cell differentiation into hepatocytes and biliary cells is dependent on a gradient of Tgf-β signaling. It is conceivable that different Tgf-β levels, coupled with Tgfbr mutation, differentially modulate distinct signaling pathways that control unique sets of downstream target genes.

In patients with TGFBR2 mutation, it is conceivable that elevated TGF-β2 signals through the TGF-βRI/RIII complex to induce a non-SMAD dependent signaling cascade. This is a significant advancement in our understanding of the mechanism of TGF-β signaling. Previous studies suggested that Tgf-βRIII appears dispensable for Tgf-β-mediated signal transduction because it lacks an intracellular domain and most cells that lack functional Tgf-βRIII still respond to Tgf-β. However, these studies failed to consider how Tgf-β signals in the absence of Tgf-βRII. We show here that a Tgf-βRIII/Tgf-βRI/β-spectrin complex can be activated by elevated Tgf-β2 and utilizes the p38 MAPK signaling cascade to regulate downstream target genes. Given our findings that elevated Tgf-β2 activity and following ectopic p38 MAPK activation is responsible for adversely affecting cell proliferation in the CNCC-derived palatal mesenchyme and that reduction of Tgfb2 or Tgfbr1/Alk5 rescues cleft palate in Tgfbr2^{fl/fl}; Wnt1-Cre mice, we propose that elevated Tgf-β2 and ectopic p38 MAPK activation might be useful diagnostic biomarkers and drug targets for the prevention and treatment of patients with congenital malformations. Given the early developmental onset of structural birth defects, modulation of TGF-β signaling at the ligand or receptor level may provide opportunities for treating individuals with altered TGF-β signaling.

Table 1 (1): Up-regulated genes in the palate of Tgfbr2^{fl/fl}; Wnt1-Cre mice at E14.5. These genes were identified with the selection criteria of genes showing >1.5-fold change with a <5% FDR. CKO refers to Tgfbr2^{fl/fl}; Wnt1-Cre mice and WT refers to Tgfbr2^{fl/fl} control mice. Log(2)-transformed gene expression scores are provided along with geometric means and FDR calculations, as described in the Methods.

Table 2: Down-regulated genes in the palate of Tgfbr2^{fl/fl}; Wnt1-Cre mice at E14.5. These genes were identified with the selection criteria of genes showing >1.5-fold change with a <5% FDR. CKO refers to Tgfbr2^{fl/fl}; Wnt1-Cre mice and WT refers to Tgfbr2^{fl/fl} control mice. Log(2)-transformed gene expression scores are provided along with geometric means and FDR calculations, as described in the Methods.

Table 3: Mass spectrometry analysis in the MEPM cells of Tgfbr2^{fl/fl}; Wnt1-Cre mice. These molecules were identified using mass spectrometer with extracts from MEPM cells of E13.5 Tgfbr2^{fl/fl} and Tgfbr2^{fl/fl}; Wnt1-Cre mice, as described in the Methods. #1 shows the approximately 30 kDa-band in FIG. 20, and #2 shows the approximately 270 kDa-band in FIG. 21.

### Materials and Methods

Animals. To generate Tgfbr2^{fl/fl}; Wnt1-Cre mice, we mated Tgfbr2^{fl/fl}; Wnt1-Cre with Tgfbr2^{fl/fl} mice. To generate Tgfbr2^{fl/fl}; Wnt1-Cre; Tgfb2^{+/-} mice, we mated Tgfbr2^{fl/+}; Wnt1-Cre; Tgfb2^{+/-} with Tgfbr2^{fl/fl} mice. To generate Tgfbr2^{fl/fl}; Wnt1-Cre; Alk5^{fl/+} mice, we mated Tgfbr2^{fl/+}; Wnt1-Cre; Alk5^{fl/+} with Tgfbr2^{fl/fl}; Alk5^{fl/fl} mice. Genotyping was performed using PCR primers as described previously.

Microarray analysis. Total RNA samples (1 μg per sample) were converted into biotin-labeled cDNA using the Enzo™BioArray™ Terminal Labeling Kit with Biotin-ddUTP and standard protocols recommended by Affymetrix (Santa Clara, Calif.). Fragmented cDNA was applied to GeneChip®, Mouse Genome 430 2.0 Arrays (Affymetrix) that contain probe sets designed to detect over 39,000 transcripts. Microarrays were hybridized, processed and scanned, as previously described using manufacturers recommended conditions. WebArray software was used to generate scaled log2 transformed gene expression values using the RMA algorithm. Probes sets showing >1.5-fold differential expression with a <5% FDR (false discovery rate) were identified through LIMMA (Liner Models for Microarray Data)-based liner model statistical analysis and FDR calculations made using the SPLOSH (spacings LOESS histogram) method. Ingenuity Pathway Analysis (IPA) software (Ingenuity Systems, Redwood City, Calif.) was also used to analyze functional relationships among differentially expressed genes. All scaled gene expression scores and .cel files are available at the National Center for Biotechnology Information (NCBI) Gene Expression Omnibus (GEO) repository http://www.ncbi.nih.gov/geo/ under Series Accession Number GSE22989.

Histological examination. Hematoxylin and eosin staining and BrdU staining were performed as described previously. Immunohistochernical staining was performed as described previously. Antibodies used for immunohistochemistry were anti-β-spectrin rabbit polyclonal, and anti-Tgf-βRIII mouse monoclonal antibodies (Santa Cruz Biotechnology), anti-Tgf-β2 mouse monoclonal, and anti-14-3-3ζ/δ rabbit polyclonal antibodies (Abeam). Fluorescence images were obtained using a fluorescence microscope (Model IX71, Olympus).

Primary MEPM cells. Primary MEPM cells were obtained from E13.5 embryos. Briefly, palatal shelves were dissected at E13.5, and trypsinized for 30 minutes at 37° C. in a CO₂ incubator. After pipetting thoroughly, cells were cultured in Dulbecco's modified Eagle's medium containing 10% fetal bovine serum supplemented with penicillin, streptomycin, L-glutamate, sodium pyruvate, and nonessential amino acids. MEPM cells were treated with or without p38 MAPK inhibitor SB203580 at 10 μM for 24 hours. For 14-3-3ζ/δ, phosho-14-3-3ζ/δ, and β-spectrin staining, MEPM cells were fixed and stained with anti-14-3-3 (Abeam), phospho-14-3-3 (ABR), and β-spectrin (Santa Cruz Biotechnology) antibodies as described previously. All fluorescence images were obtained using a fluorescence microscope (Model IX71, Olympus). Pictures were taken using MicroSuite Analytical Suite software (Olympus).

Immunological analysis. Immunoblots were performed as described previously. Antibodies used for immunoblotting were as follows: rabbit polyclonal antibodies against p38, JNK, and phospho-JNK (Cell Signaling Technology), 14-3-3ζ/δ, Smad2/3, Tgf-βRI, and Tgf-βRII (Abeam), phospho-14-3-3 (ABR), 14-3-3ε, and β-spectrin (Santa Cruz Biotechnology); rabbit monoclonal antibodies against phospho-p38, and phospho-Smad2 (Cell Signaling Technology); and mouse monoclonal antibodies against Smad4, and Tgf-βRIII (Santa Cruz Biotechnology), Tgf-β2 (Abeam), and GAPDH (Chemicon).

Immunoprecipitation. Extracts in cell lysis buffer containing 10 mM Tris (pH 7.5), 100 mM NaCl, 1% Triton X-100, and protease inhibitor cocktail (Complete™, Invitrogen) were lysed by sonication. Lysates containing an equal amount of protein (total volume 1 ml) were centrifuged at 1,000×g for 5 minutes at 4° C. to remove debris. The supernatant was precleared with 25 μl of protein A-agarose (20% slurry, Santa Cruz Biotechnology). 40 μl of protein A-agarose and 2 μg of anti-Tgf-β2, Tgf-βRI (Abeam), Tgf-βRIII, or β-spectrin (Santa Cruz Biotechnology) antibody were then added to the lysate, and the mixture was rotated for 12 hours at 4° C. The immunoprecipitate-agarose complex was washed five times with ice-cold cell lysis buffer. The complex was then boiled for 5 minutes in SDS sample buffer in the presence of β-mercaptoethanol to elute proteins and centrifuged at 1,000×g for 5 minutes at 4° C. The supernatant was subjected to SDS-PAGE, transferred to a polyvinylidene difiuoride membrane, and analyzed by immunoblotting with indicated antibodies.

Affinity cross-linking. Cells were incubated with I¹²⁵-labeled Tgf-β (1 ng/ml) in a binding buffer [128 mM NaCl, 5 mM KCl, 1.2 mM CaCl₂, 5 mM MgSO₄, 50 mM Hepes buffer (pH 7.4)]. After incubation for 30 minutes on ice, cross-linker solution (200 μM Disuccinimidyl suberate in binding buffer) was added for 30 minutes on ice. The reaction was stopped by a detachment buffer [0.25 M Sucrose, 1 mM EDTA, 10 mM Tris buffer (pH 7.4)]. The precipitate was dissolved in SDS/PAGE sample buffer, boiled for 5 min and applied to a SDS-PAGE gel.

Quantitative RT-PCR. Total RNA was isolated from mouse embryonic palate dissected at each developmental stage with the QIAshredder and RNeasy Mini extraction kit (QIAGEN). cDNA was synthesized from lug of total RNA using Superscript III (Invitrogen), and quantitative PCR was performed by SYBR Green (Bio-Rad Laboratories) in an iCycler (Bio-Rad Laboratories), as described previously. PCR primers are available upon request. Two-tailed Student's t-test was applied for statistical analysis of qPCR data. A p value ≦0.05 was considered statistically significant. For all graphs, data are represented as mean±SD.

Palatal shelf organ culture. Time-pregnant mice were sacrificed at E13.5. Genotyping was carried out as described above. The palatal shelves were microdissected and cultures in serum-less chemically defined medium as previously described. After 24 hours in culture treated with p38 MAPK inhibitor SB203580 (100 μM), and neutralizing antibody for Tgf-β2 (2 μg/ml) or Tgf-βRIII (2 μg/ml), palates were harvested, fixed in 4% paraformaldehyde/0.1 M phosphate buffer (pH 7.4) and processed.

Mass spectrometry; Trypsin digestion and liquid chromatography tandem mass spectrometry (LC-MS/MS). Samples were separated by 1-D polyacrylamide gel, and stained with Coomassie Blue to visualize band. Protein bands were excised and tryptic digests were analyzed in the University of Southern California, Proteomics Core Facility by LC-MS/MS, as described previously. Mass analysis was done using a ThermoFinnigan LCQ Deca XP Plus ion trap mass spectrometer equipped with a nanospray ion source using a 4.5 cm long metal needle (Hamilton; 950-00954) in a data-dependent acquisition mode. Protein identification was performed with the MS/MS search software Mascot 1.9 (Matrix Science, Boston, Mass.) with confirmatory or complementary analyses with TurboSequest as implemented in the Bioworks Browser 3.2, build 41 (ThermoFinnigan).

Whole-mount skeletal staining. The three-dimensional architecture of the skeleton was examined using a modified whole-mount Alcian blue-Alizarin Red S staining protocol as previously described.

### Example 3

### Lipid Metabolism and Craniofacial Malformation

Recent findings indicate that Smad-independent MAPK pathways [p38 and c-Jun NH₂-terminal kinase (JNK)] are activated by Tgf-β receptor kinase-independent pathway via ubiquitination and phosphorylation of Tgf-β-activated kinase 1 (Tak1) after binding of Tgf-β ligands. We hypothesized that the Tgf-β2/Tgf-βRIII/Tgf-βRI/β-spectrin complex might activate Smad-independent Tgf-β signaling using a Tak1 cascade. Indeed, ubiquitination and phosphorylation of Tak1 were up-regulated in Tgfbr2^{fl/fl}; Wnt1-Cre MEPM cells (FIG. 24d). Thus, our results show that, in the absence of Tgfbr2, elevated Tgf-β2 forms a complex with Tgf-βRIII/Tgf-βRI/β-spectrin and induces a p38 MAPK signaling cascade in the palatal mesenchyme (FIG. 24e).

Next, we investigated the functional significance of Smad-independent Tgf-β signaling in the palatal mesenchyme. Strikingly, primary MEPM cells from Tgfbr2^{fl/fl}; Wnt1-Cre mice spontaneously accumulated intracellular lipid droplets in regular medium (FIGS. 25a,b and FIG. 34h). To analyze lipid metabolic aberrations in vivo, we assayed the amount of triacylglycerol in the palates of Tgfbr2^{fl/fl}; Wnt1-Cre and Tgfbr2^{fl/fl} mice at E18.5. The triacylglycerol level was significantly increased in Tgfbr2^{fl/fl}; Wnt1-Cre palates at E18.5 (FIG. 25c). Furthermore, we detected lipid droplet accumulation in the craniofacial region of Tgfbr2^{fl/fl}; Wnt1-Cre mice at E14.5 and E18.5 by Oil Red O staining (FIG. 25d) and transmission electron microscopic analyses (FIG. 25e and FIG. 34h).

To test whether activation of p38 MAPK pathway via Tgf-βRI/RIII caused lipid droplet accumulation in Tgfbr2^{fl/fl}; Wnt1-Cre MEPM cells, we used p38 MAPK inhibitors, SB203580 and PD169316. After the treatment of MEPM cells with p38 MAPK inhibitors, we failed to detect lipid droplet accumulation in Tgfbr2^{fl/fl}; Wnt1-Cre MEPM cells (FIG. 25f and FIG. 34i). These data indicate that up-regulated p38 MAPK triggers a sequence of lipid accumulation in Tgfbr2^{fl/fl}; Wnt1-Cre MEPM cells.

Elevated Tgf-β2 expression in Tgfbr2^{fl/fl}; Wnt1-Cre mice activates a Smad-independent Tgf-β signaling cascade and causes lipid accumulation and may adversely affect CNCC proliferation during palatogenesis. Therefore, reduction of Tgf-β2 would increase CNCC proliferation and might prevent the cleft palate in Tgfbr2^{fl/fl}; Wnt1-Cre mice. To test this hypothesis, we generated Tgfbr2^{fl/fl}; Wnt1-Cre; Tgfb2^{+/-} mice. Indeed, Tgfbr2^{fl/fl}; Wnt1-Cre; Tgfb2^{+/-} mice formed palates normally with 82.5% phenotype penetrance (33/40) (FIG. 26a and FIGS. 35, 36). Furthermore, the up-regulated p38/14-3-3 MAPK initially detected in Tgfbr2^{fl/fl}; Wnt1-Cre palates was restored to control levels in E14.5 Tgfbr2^{fl/fl}; Wnt1-Cre; Tgfb2^{+/-} palates (FIG. 26b). In addition, lipid accumulation and cell proliferation activity in the palatal mesenchyme were restored to control levels in Tgfbr2^{fl/fl}; Wnt1-Cre; Tgfb2^{+/-} palates (FIG. 26c and FIG. 35). In order to test further the hypothesis that activation of alternative Tgf-β signaling in Tgfbr2^{fl/fl}; Wnt1-Cre mice depends on the presence of Tgfbr1(Alk5) and Tgfbr3, we generated Tgfbr2^{fl/fl}; Wnt1-Cre; Alk5^{fl/+} mice. Significantly, a reduction of Tgfbr1 corrected the lipid metabolic aberrations, restored cell proliferation, and rescued the cleft palate phenotype in Tgfbr2^{fl/fl}; Wnt1-Cre; Alk5^{fl/+} mice with 91.7% penetrance (22/24) (FIGS. 25d,e and FIG. 37 and data not shown). Our findings indicate that elevated Smad-independent p38 MAPK activation mediated through Tgf-βRI/RIII is responsible for the CNCC proliferation defect and failure of palatal fusion in Tgfbr2^{fl/fl}; Wnt1-Cre mice.

An antihypertensive medication that acts as an angiotensin II receptor blocker (ARB), losartan, prevents the enlarged aorta in patients of Marfan syndrome by suppressing up-regulated angiotensin-mediated Tgf-β signaling. In addition, recent studies reveal that telmisartan, another ARB, has an unexpected additional function that improves lipid metabolism. The IUPAC name for telmisartan is 2-(4-{[4-methyl-6-(1-methyl-1H-1,3-benzodiazol-2-yl)-2-propyl-1H-1,3-benzodiazol-1-yl]methyl}phenyl)benzoic acid. In our microarray analysis (see above), we found up-regulated gene expression of angiotensinogen and thrombospondin 1, which are also up-regulated in Marfan syndrome, in Tgfbr2^{fl/fl}; Wnt1-Cre mice (FIG. 38). Therefore, we hypothesized it might be possible to rescue cleft palate using telmisartan in Tgfbr2^{fl/fl}; Wnt1-Cre mice. Indeed, telmisartan rescued cleft palate in 28% of Tgfbr2^{fl/fl}; Wnt1-Cre mice, without obvious side-effects in other organs (5/18 in Tgfbr2^{fl/fl}; Wnt1-Cre mice) (FIG. 26f and FIG. 39). Up-regulated p38 MAPK activity and increased triacylglycerol level in Tgfbr2^{fl/fl}; Wnt1-Cre mice were restored to normal by telmisartan treatment (FIGS. 26g,h). Thus, the reduction of up-regulated p38 MAPK activity and improvement of lipid metabolism restored cell proliferation defect and rescued cleft palate in about 30% of the Tgfbr2^{fl/fl}; Wnt1-Cre mice following telmisartan treatment. Taken together, we conclude that loss of Tgfbr2 causes lipid metabolic aberrations that adversely affect cell proliferation during palatogenesis, and importantly medications that affect lipid metabolism might be useful for the prevention of craniofacial anomalies during embryogenesis.

This study identifies a novel Tgf-β signaling mechanism and demonstrates its functional significance in regulating lipid metabolism and cell proliferation in the absence of Tgfbr2. Up-regulation of Tgfb2 and Tgfbr3 in the absence of Tgfbr2 may represent a feedback loop, whereby interrupted Tgfbr2-mediated signaling leads to an increased production of the Tgf-β ligand and signaling through an alternative Tgf-β receptor (i.e. Tgf-βRIII). Relevant to the latter observation, Tgf-β2 has a high affinity for Tgf-βRIII, a unique feature that distinguishes Tgf-β2 from other Tgf-β ligands. Previous studies suggested that Tgf-βRIII appears dispensable for Tgf-β-mediated signal transduction because it lacks an intracellular domain and most cells that lack functional Tgf-βRIII still respond to Tgf-β. However, these studies failed to consider how Tgf-β signals in the absence of Tgf-βRII. We show here that a Tgf-βRIII/Tgf-βRI complex can be activated by Tgf-β2 and utilizes a p38 MAPK signaling cascade to regulate downstream target genes.

The connection between altered Tgf-β signaling, lipid metabolic aberrations, and cell proliferation defect in CNCC offers a new perspective on the cellular mechanism of craniofacial deformities and may represent a more generalized cause of cellular defect in patients with craniofacial malformations. Triacylglycerol, which is a main component of lipid droplets, is first degraded into diacylglycerol by triglyceride lipase, then further degraded into monoacylglycerol by hormone-sensitive lipase, and finally degraded by monoglyceride lipase into glycerol. We have discovered that gene expression of monoglyceride lipase (Mgll) is significantly and specifically down-regulated in Tgfbr2^{fl/fl}; Wnt1-Cre MEPM cells (Tgfbr2^{fl/fl}; Wnt1-Cre MEPM cells versus Tgfbr2^{fl/fl} MEPM cells=6.12 fold change, p=0.0005) by both microarray analysis and quantitative RT-PCR (data not shown). Our results demonstrate that lipid metabolic aberrations may be caused by decreased Mgll expression and that Tgf-β-mediated Mgll expression is crucial for lipid metabolism in CNCC. Given our findings that elevated Smad-independent pathway is responsible for adversely affecting lipid metabolism and cell proliferation in the CNCC-derived palatal mesenchyme and that reduction of Tgfb2 or Tgfbr1/Alk5 rescues palatal fusion in Tgfbr2^{fl/fl}; Wnt1-Cre mice, we propose that Tgf-β2 or p38 MAPK activation might be a useful diagnostic biomarker and drug target for the prevention and treatment of patients with congenital malformations.

### Tables

Table 4. Up-regulated genes in the palate of Tgfbr2^{fl/fl}; Wnt1-Cre mice at E14.5. These genes were identified with the selection criteria of genes showing >1.5-fold change with a <5% FDR. CKO refers to Tgfbr2^{fl/fl}; Wnt1-Cre mice and WT refers to Tgfbr2^{fl/fl} control mice. Log(2)-transformed gene expression scores are provided along with geometric means and FDR calculations, as described in the Methods.

Table 5. Down-regulated genes in the palate of Tgfbr2^{fl/fl}; Wnt1-Cre mice at E14.5. These genes were identified with the selection criteria of genes showing >1.5-fold change with a <5% FDR. CKO refers to Tgfbr2^{fl/fl}; Wnt1-Cre mice and WT refers to Tgfbr2^{fl/fl} control mice. Log(2)-transformed gene expression scores are provided along with geometric means and FDR calculations, as described in the Methods.

Table 6. Mass spectrometry analysis in the MEPM ceils of Tgfbr2^{fl/fl}; Wnt1-Cre mice. These molecules were identified using mass spectrometry with extracts from MEPM cells of E13.5 Tgfbr2^{fl/fl} and Tgfbr2^{fl/fl}; Wnt1-Cre mice, as described in the Methods. #1 shows the approximately 30 kDa-band in FIG. 32, and #2 shows the approximately 270 kDa-band in FIG. 32.

### Methods

Animals. To generate Tgfbr2^{fl/fl}; Wnt1-Cre mice, we mated Tgfbr2^{fl/+}; Wnt1-Cre with Tgfbr2^{fl/fl} mice. To generate Tgfbr2^{fl/fl}; Wnt1-Cre; Tgfb2^{+/-} mice, we mated Tgfbr2^{fl/+}; Wnt1-Cre; Tgfb2^{+/-} with Tgfbr2^{fl/fl} mice. To generate Tgfbr2^{fl/fl}; Wnt1-Cre; Alk5^{fl/+} mice, we mated Tgfbr2^{fl/+}; Wnt1-Cre; Alk5^{fl/fl} with Tgfbr2^{fl/fl}; Alk5^{fl/fl} mice. Genotyping was performed using PCR primers as described previously^{28, 29}. Female Tgfbr2^{fl/fl} mice, which underwent timed matings with Tgfbr2^{fl/+}; Wnt1-Cre male mice, were treated with oral telmisartan (0.5 mg in 25 μl dimethyl sulfoxide per day by a feeding tube) or vehicle (25 μl dimethl sulfoxide by a feeding tube) from 0.5 day post-coitum (E0.5). Therapy was continued throughout pregnancy.

Microarray analysis. Total RNA samples (1 μg per sample) were converted into biotin-labeled cDNA using the Enzo™BioArray™ Terminal Labeling Kit with Biotin-ddUTP and standard protocols recommended by Affymetrix (Santa Clara, Calif.). Fragmented cDNA was applied to GeneChip®, Mouse Genome 430 2.0 Arrays (Affymetrix) that contain probe sets designed to detect over 39,000 transcripts. Microarrays were hybridized, processed and scanned, as previously described using manufacturer's recommended conditions. Web Array software was used to generate scaled log2 transformed gene expression values using the RMA algorithm. Probes sets showing >1.5-fold differential expression with a <5% FDR (false discovery rate) were identified through LIMMA (Liner Models for Microarray Data)-based liner model statistical analysis and FDR calculations made using the SPLOSH (spacings LOESS histogram) method. Ingenuity Pathway Analysis (IPA) software (Ingenuity Systems, Redwood City, Calif.) was also used to analyze functional relationships among differentially expressed genes. All scaled gene expression scores and .cel files are available at the National Center for Biotechnology Information (NCBI) Gene Expression Omnibus (GEO) repository http://www.ncbi.nih.gov/geo/ under Series Accession Number GSE22989.

Histological examination. Cryosections were stained with Oil Red O to detect lipid production. Prepared sections were washed three times in phosphate buffer saline (PBS) (each wash 5 min) and fixed in 4% formaldehyde in 0.1 M phosphate buffer for 1 h at room temperature. Sections were then incubated for 15 min in 60% isopropanol and stained for 15 min with a filtered solution of three parts of Oil Red O (saturated in isopropanol) and two parts of ddH₂O. For the next step, slides were briefly rinsed in 60% isopropanol and washed thoroughly in ddH₂O. Cells were counterstained in Mayers Hematoxylin Solution.

Electron microscopy. The fixative consisted of 2% paraformaldehyde, 1% glutaraldehyde, and 0.07 M cacodylate buffer (pH 7.4). Samples were post-fixed with 1% reduced osmium tetroxide for 1 h. All tissue slices were dehydrated in graded series of ethanol, embedded in Epon 812, and then sectioned for electron microscopic observation.

Triacylglycerol amount. Triacylglycerol amounts were measured by an enzymatic method using N-ethyl-N-(2-hydroxy-3-sulfopropyl)-3,5-dimethoxyaniline sodium salt according to the manufacturer's instructions (Wako). Data is expressed as mg per 100 mg protein.

Primary MEPM cells. Primary MEPM cells were obtained from E13.5 embryos. Briefly, palatal shelves were dissected at E13.5, and trypsinized for 30 minutes at 37° C. in a CO₂ incubator. After pipetting thoroughly, cells were cultured in Dulbecco's modified Eagle's medium containing 10% fetal bovine serum supplemented with penicillin, streptomycin, L-glutamate, sodium pyruvate, and nonessential amino acids. MEPM cells were treated with or without p38 MAPK inhibitor SB203580 or PD169316 at 10 μM for three weeks to investigate whether lipid droplets accumulated.

Immunological analysis. Immunoblots were performed as described previously. Antibodies used for immunoblotting were as follows: rabbit polyclonal antibodies against p38, JNK, and phospho-JNK (Cell Signaling Technology), 14-3-3ζ/δ, Smad2/3, Tgf-βrI, and Tgf-βrII (Abeam), phospho-14-3-3 (ABR), 14-3-3ε, and β-spectrin (Santa Cruz Biotechnology); rabbit monoclonal antibodies against phospho-p38, and phospho-Smad2 (Cell Signaling Technology); and mouse monoclonal antibodies against Smad4, and Tgf-βrIII (Santa Cruz Biotechnology), Tgf-β2 (Abeam), and GAPDH (Chemicon). MEPM cells were treated with or without p38 MAPK inhibitor SB203580 at 10 μM for 24 hours.

Immunoprecipitation. Extracts in cell lysis buffer containing 10 mM Tris (pH 7.5), 100 mM NaCl, 1% Triton X-100, and protease inhibitor cocktail (Complete™, Invitrogen) were lysed by sonication. Lysates containing an equal amount of protein (total volume 1 ml) were centrifuged at 1,000×g for 5 minutes at 4° C. to remove debris. The supernatant was precleared with 25 μl of protein A-agarose (20% slurry, Santa Cruz Biotechnology). 40 μl of protein A-agarose and 2 μg of anti-Tgf-β2, Tgf-βrI (Abeam), Tgf-βrIII, or β-spectrin (Santa Cruz Biotechnology) antibody were then added to the lysate, and the mixture was rotated for 12 hours at 4° C. The Immunoprecipitate-agarose complex was washed five times with ice-cold cell lysis buffer. The complex was then boiled for 5 minutes in SDS sample buffer in the presence of β-mercaptoethanol to elute proteins and centrifuged at 1,000×g for 5 minutes at 4° C. The supernatant was subjected to SDS-PAGE, transferred to a polyvinylidene difluoride membrane, and analyzed by immunoblotting with indicated antibodies.

Affinity cross-linking. Cells were incubated with I¹²⁵-labeled Tgf-β (1 ng/ml) in a binding buffer [128 mM NaCl, 5 mM KCl, 1.2 mM CaCl₂, 5 mM MgSO₄, 50 mM Hepes buffer (pH 7.4)]. After incubation for 30 minutes on ice, cross-linker solution (200 μM Disuccinimidyl suberate in binding buffer) was added for 30 minutes on ice. The reaction was stopped by a detachment buffer [0.25 M Sucrose, 1 mM EDTA, 10 mM Tris buffer (pH 7.4)]. The precipitate was dissolved in SDS/PAGE sample buffer, boiled for 5 min and applied to a SDS-PAGE gel.

Quantitative RT-PCR. Total RNA was isolated from mouse embryonic palate dissected at each developmental stage with the QIAshredder and RNeasy Mini extraction kit (QIAGEN). cDNA was synthesized from 1 μg of total RNA using Superscript III (Invitrogen), and quantitative PCR was performed by SYBR Green (Bio-Rad Laboratories) in an iCycler (Bio-Rad Laboratories), as described previously. PCR primers are available upon request. Two-tailed Student's t-test was applied for statistical analysis of qPCR data. A p value ≦0.05 was considered statistically significant. For all graphs, data are represented as mean±SD.

Statistical analysis. Two-tailed Student's t-test was applied for statistical analysis. For all graphs, data are represented as mean±SD. A p value of less than 0.05 was considered statistically significant.

Immunological analysis. Immunoblots were performed as previously. Antibodies used for immunoblotting were as follows: rabbit polyclonal antibodies against phospho-JNK, phospho-Erk, Akt, phospho-Akt (Ser473), phospho-Akt (Thr308), phospho-PTEN, phospho-PDK1 (Cell Signaling Technology); and mouse monoclonal antibody against p38, INK, Erk (Cell Signaling Technology).

Histological examination. Hematoxylin and eosin staining and BrdU staining were performed as described previously. Immunohistochemical staining was performed as described previously. Antibodies used for immunohistochemistry were anti-β-spectrin rabbit polyclonal, and anti-Tgf-βrIII mouse monoclonal antibodies (Santa Cruz Biotechnology), anti-Tgf-β2 mouse monoclonal, and anti-14-3-3 rabbit polyclonal antibodies (Abeam). Fluorescence images were obtained using a fluorescence microscope (Model IX71, Olympus).

Mass spectrometry; Trypsin digestion and liquid chromatography tandem mass spectrometry (LC-MS/MS). Samples were separated by 1-D polyacrylamide gel, and stained with Coomassie Blue to visualize bands. Protein bands were excised and tryptic digests were analyzed in the University of Southern California, Proteomics Core Facility by LC-MS/MS as described previously³⁹. Mass analysis was done using a ThermoFinnigan LCQ Deca XP Plus ion trap mass spectrometer equipped with a nanospray ion source using a 4.5 cm long metal needle (Hamilton; 950-00954) in a data-dependent acquisition mode. Protein identification was performed with the MS/MS search software Mascot 1.9 (Matrix Science, Boston, Mass.) with confirmatory or complementary analyses with TurboSequest as implemented in the Bioworks Browser 3.2, build 41 (ThermoFinnigan).

Adipogenic differentiation. Adipogenic differentiation was induced by culture in monolayers for one week after initial seeding of the cells at 1.5×10⁴ cells/cm² in complete medium supplemented with 1 μmol/L dexamethasone, 1 μg/mL insulin, and 0.5 mmol/L 3-isobutyl-1-methylxantine. Lipid droplets that accumulated within cells after adipogenic differentiation were stained by Oil Red O reagent. Lipids were stained with Oil Red O, dissolved in isopropanol, and then measured in a spectrometer. Data is expressed as results at OD490 nm. Quantitative analyses of glycerol released into the medium by isoproterenol stimulation were performed following the procedure in the Adipolysis Assay kit (Cayman, Mich.).

Marking the progeny of CNCC. Mating Wnt1-Cre and R26R mice generated transgenic mice with neural crest progeny cells that could be indelibly labeled with β-galactosidase. β-galactosidase (LacZ) activity was detected by staining sections according to standard procedures as described previously.

Flow cytometry analysis. Cell sorting was performed on a FACScan (Becton Dickinson) with Cellquest software. Briefly, 1×10⁶ cells were stained with 1 mM fluorescein di-β-d-galactopyranoside (BD Pharmingen, San Jose, Calif.) at 37° C. for 1 min. The reaction was stopped by adding 0.8 ml of ice-cold fluorescence-activated cell sorting (FACS) buffer.

Purification of CNCC-derived MEPM cells. The palatal shelves were dissected at E13.5, and the CNCC-derived MEPM cells were purified using cell sorting by fluorescein di-β-d-galactopyranoside to isolate cells carrying the R26R reporter gene. For 14-3-3ζ/δ, phosho-14-3-3, and β-spectrin staining, MEPM cells were fixed and stained with anti-14-3-3ζ/δ (Abeam), phospho-14-3-3 (ABR), and β-spectrin (Santa Cruz Biotechnology) antibodies as described previously. All fluorescence images were obtained using a fluorescence microscope (Model IX71, Olympus). Pictures were taken using MicroSuite Analytical Suite software (Olympus).

Pulse-chase analysis of lipid droplet formation. Chemical inducers, oleic acid for lipogenesis and isoproterenol for lipolysis, were added into culture medium to analyze lipid droplet formation. The number of lipid droplets per cell was counted. Oleic acid was added to the medium at 100 μM for 0, 60, and 120 min, and then washed out and changed to the medium with 100 μM isoproterenol for 10 and 30 min. Twenty cells were counted at each time point.

Whole-mount skeletal staining. The three-dimensional architecture of the skeleton was examined using a modified whole-mount Alcian blue-Alizarin Red S staining protocol as described previously.

TABLE 1
Upregulated genes in the palate of Tgfbr2^{fl/fl}; Wnt1-Cre mice at E14.5

| | | | CKO | WT | | |
|---|---|---|---|---|---|---|
| | | | Geo | Geo | CKO/WT | |
| Symbol | GeneID | Gene Title | Mean | Mean | Ratio | FDR |
| Sned1 | 208777 | sushi, nidogen and EGF-like domains 1 | 1668 | 417 | 4.00 | 0.001 |
| — | — | — | 315 | 81 | 3.88 | 0.013 |
| Meig1 | 104362 | melosis expressed gene 1 | 270 | 77 | 3.51 | 0.009 |
| — | — | — | 204 | 62 | 3.30 | 0.020 |
| Krt13 | 16663 | keratin 13 | 389 | 129 | 3.02 | 0.023 |
| 4833427G06Rik | 235345 | RIKEN cDNA 4833427G06 gene | 310 | 109 | 2.84 | 0.007 |
| Hdc | 15186 | histidine decarboxylase | 172 | 62 | 2.78 | 0.009 |
| Lrrc23 | 16977 | leucine rich repeat containing 23 | 651 | 243 | 2.68 | 0.003 |
| Dynlrb2 | 75465 | dynein light chain roadblock-type 2 | 1287 | 482 | 2.67 | 0.012 |
| — | — | — | 35 | 13 | 2.63 | 0.023 |
| Ccdc153 | 270150 | coiled-coil domain containing 153 | 103 | 40 | 2.58 | 0.006 |
| Fam154b | 330577 | family with sequence similarity 154, member B | 86 | 33 | 2.57 | 0.001 |
| Dnahc12 /// Dnahc7l | 110083 /// | dynein, axonemal, heavy chain 12 /// dynein, axonemal, | 77 | 30 | 2.55 | 0.019 |
| | 207778 | heavy chain 7-like | | | | |
| C030048H21Rik | 77481 | RIKEN cDNA C030048H21 gene | 111 | 44 | 2.52 | 0.005 |
| — | — | — | 121 | 50 | 2.43 | 0.002 |
| Morn5 | 75495 | MORN repeat containing 5 | 191 | 79 | 2.40 | 0.010 |
| Ak7 | 78801 | adenylate kinase 7 | 203 | 86 | 2.36 | 0.002 |
| 9630021D06Rik | 319926 | RIKEN cDNA 9630021D06 gene | 284 | 122 | 2.32 | 0.024 |
| — | — | — | 261 | 113 | 2.30 | 0.013 |
| Hdc | 15186 | histidine decarboxylase | 235 | 103 | 2.29 | 0.010 |
| Ccdc108 | 241116 | coiled-coil domain containing 108 | 104 | 46 | 2.28 | 0.000 |
| E030011K20Rik | 208613 | RIKEN cDNA E030011K20 gene | 99 | 44 | 2.28 | 0.034 |
| Rsph1 | 22092 | radial spoke head 1 homolog (Chlamydomonas) | 393 | 173 | 2.27 | 0.022 |
| — | — | — | 407 | 179 | 2.27 | 0.044 |
| 1110069O07Rik | 71798 | RIKEN cDNA 1110069O07 gene | 327 | 144 | 2.27 | 0.004 |
| Wdr63 | 242253 | WD repeat domain 63 | 89 | 40 | 2.26 | 0.017 |
| 4921509J17Rik | 70857 | RIKEN cDNA 4921509J17 gene | 291 | 132 | 2.20 | 0.011 |
| Ccdc67 | 234964 | coiled-coil domain containing 67 | 952 | 439 | 2.17 | 0.031 |
| 1700012B09Rik | 69325 | RIKEN cDNA 1700012B09 gene | 105 | 49 | 2.16 | 0.009 |
| Agt | 11606 | angiotensinogen (serpin peptidase inhibitor, clade A, | 179 | 83 | 2.16 | 0.000 |
| | | member 8) | | | | |
| Ube2d2 | 56550 | ubiquitin-conjugating enzyme E2D 2 | 196 | 91 | 2.16 | 0.019 |
| — | — | — | 114 | 53 | 2.16 | 0.042 |
| 1110069O07Rik | 71798 | RIKEN cDNA 1110069O07 gene | 316 | 147 | 2.15 | 0.001 |
| Ttc29 | 73301 | tetratricopeptide repeat domain 29 | 56 | 26 | 2.14 | 0.033 |
| Tekt4 | 71840 | tektin 4 | 132 | 62 | 2.11 | 0.032 |
| Dnali1 | 75563 | dynein, axonemal, light intermediate polypeptide 1 | 148 | 70 | 2.10 | 0.003 |
| Vit | 74199 | vitrin | 166 | 79 | 2.09 | 0.000 |
| Tgfb2 | 21808 | transforming growth factor, beta 2 | 295 | 143 | 2.07 | 0.001 |
| Krt13 | 16663 | keratin 13 | 25 | 12 | 2.02 | 0.001 |
| Ndufa4l2 | 407790 | NADH dehydrogenase (ubiquinone) 1 alpha subcomplex, | 1080 | 534 | 2.02 | 0.001 |
| | | 4-like 2 | | | | |
| 1700009P17Rik | 75472 | RIKEN cDNA 1700009P17 gene | 723 | 360 | 2.01 | 0.040 |
| Pde4d | 238871 | phosphodiesterase 4D, cAMP specific | 43 | 22 | 2.00 | 0.048 |
| Zmynd10 | 114602 | zinc finger, MYND domain containing 10 | 239 | 121 | 1.98 | 0.032 |
| Dnahc2 | 327954 | dynein, axonemal, heavy chain 2 | 128 | 65 | 1.97 | 0.008 |
| Traf1 | 22029 | TNF receptor-associated factor 1 | 78 | 40 | 1.96 | 0.000 |
| Ube3a | 22215 | ubiquitin protein ligase E3A | 205 | 105 | 1.96 | 0.033 |
| Tgfb2 | 21808 | transforming growth factor, beta 2 | 1262 | 645 | 1.95 | 0.002 |
| 6820408C15Rik | 228778 | RIKEN cDNA 6820408C15 gene | 178 | 92 | 1.94 | 0.009 |
| 1700094D03Rik | 73545 | RIKEN cDNA 1700094D03 gene | 570 | 296 | 1.92 | 0.002 |
| Pde4d | 238871 | phosphodiesterase 4D, cAMP specific | 43 | 22 | 2.00 | 0.048 |
| Zmynd10 | 114602 | zinc finger, MYND domain containing 10 | 219 | 121 | 1.98 | 0.032 |
| Dnahc2 | 327954 | dynein, axonemal, heavy chain 2 | 128 | 65 | 1.97 | 0.008 |
| Traf1 | 22029 | TNF receptor-associated factor 1 | 78 | 40 | 1.96 | 0.000 |
| Ube3a | 22215 | ubiquitin protein ligase E3A | 205 | 105 | 1.96 | 0.033 |
| Tgfb2 | 21808 | transforming growth factor, beta 2 | 1262 | 645 | 1.95 | 0.002 |
| 6820408C15Rik | 228778 | RIKEN cDNA 6820408C15 gene | 178 | 92 | 1.94 | 0.009 |
| 1700094D03Rik | 73545 | RIKEN cDNA 1700094D03 gene | 570 | 296 | 1.92 | 0.002 |
| Slitrk1 | 76965 | SLIT and NTRK-like family, member 1 | 104 | 54 | 1.92 | 0.017 |
| 4933404M02Rik | 66748 | RIKEN cDNA 4933404M02 gene | 54 | 28 | 1.92 | 0.004 |
| Grid2 | 14804 | glutamate receptor, ionotropic, delta 2 | 128 | 68 | 1.89 | 0.001 |
| Krt4 | 16682 | keratin 4 | 356 | 188 | 1.89 | 0.039 |
| Krt4 | 16682 | keratin 4 | 282 | 149 | 1.89 | 0.006 |
| AbcaBa | 217258 | ATP-binding cassette, sub-family A (ABC1), member 8a | 337 | 179 | 1.88 | 0.000 |
| Akap14 | 434756 | A kinase (PRKA) anchor protein 14 | 81 | 43 | 1.88 | 0.012 |
| — | — | — | 150 | 80 | 1.88 | 0.044 |
| D19Ertd652e | 70806 | DNA segment, Chr 19, ERATO Doi 652, expressed | 74 | 40 | 1.87 | 0.000 |
| D19Ertd652e | 70806 | DNA segment, Chr 19, ERATO Doi 652, expressed | 25 | 13 | 1.87 | 0.011 |
| Tekt1 | 21689 | tektin 1 | 373 | 199 | 1.87 | 0.039 |
| Psma3 | 19167 | Proteasome (prosome, macropain) subunit, alpha type 3 | 207 | 112 | 1.84 | 0.013 |
| Tppp3 | 67971 | tubulin polymerization-promoting protein family | 608 | 330 | 1.84 | 0.036 |
| | | member 3 | | | | |
| — | — | — | 87 | 47 | 1.84 | 0.010 |
| Nme5 | 75533 | non-metastatic cells 5, protein expressed in | 401 | 239 | 1.83 | 0.032 |
| | | (nucleoside-diphosphate kinase) | | | | |
| Serpine1 | 18787 | serine (or cysteine) peptidase inhibitor, clade E, | 90 | 49 | 1.83 | 0.000 |
| | | member 1 | | | | |
| Dnahc7b | 227058 | dynein, axonemal, heavy chain 78 | 87 | 48 | 1.82 | 0.011 |
| 6430537H07Rik | 226265 | RIKEN cDNA 6430537H07 gene | 135 | 75 | 1.81 | 0.040 |
| Mdh1b | 76668 | malate dehydrogenase 1B, NAD (soluble) | 45 | 25 | 1.81 | 0.037 |
| C230072F16Rik | 320784 | RIKEN cDNA C230072F16 gene | 76 | 42 | 1.80 | 0.004 |
| Hspa4l | 18415 | heat shock protein 4 like | 326 | 182 | 1.79 | 0.017 |
| 1600029D21Rik | 76509 | RIKEN cDNA 1600029D21 gene | 167 | 93 | 1.79 | 0.020 |
| Krt13 | 16663 | keratin 13 | 62 | 35 | 1.79 | 0.050 |
| Cdh7 | 241201 | cadherin 7, type 2 | 115 | 64 | 1.78 | 0.014 |
| 4932425I24Rik | 320214 | RIKEN cDNA 4932425I24 gene | 63 | 35 | 1.78 | 0.010 |
| Adamts5 /// | 100048332 | a disintegrin-like and metallopeptidase (reprolysin type) | 328 | 185 | 1.78 | 0.000 |
| LOC100048332 | /// 23794 | with thrombospondin type 1 motif, 5 (aggrecanase-2) | | | | |
| | | /// similar to a disintegrin-like and metalloprotease | | | | |
| | | (reprolysin type) with thrombospondin type 1 motif, 5 | | | | |
| | | (aggrecanase-2) | | | | |
| 2900006K08Rik | 72873 | RIKEN cDNA 2900006K08 gene | 155 | 87 | 1.78 | 0.041 |
| A330021E22Rik | 207686 | RIKEN cDNA A330021E22 gene | 174 | 98 | 1.77 | 0.001 |
| D14Abb1e | 218850 | DNA segment, Chr 14, Abbott 1 expressed | 383 | 216 | 1.77 | 0.028 |
| Tgfb2 | 21808 | transforming growth factor, beta 2 | 957 | 544 | 1.76 | 0.000 |
| Dio3os | 353504 | deiodinase, iodothyronine type III, opposite strand | 384 | 218 | 1.76 | 0.000 |
| Hspa4l | 18415 | heat shock protein 4 like | 112 | 64 | 1.75 | 0.012 |
| Kndc1 | 76484 | kinase non-catalytic C-lobe domain (KIND) containing 1 | 108 | 62 | 1.75 | 0.007 |
| 1600029D21Rik | 76509 | RIKEN cDNA 1600029D21 gene | 130 | 75 | 1.74 | 0.015 |
| — | — | — | 66 | 38 | 1.74 | 0.003 |
| Mamdc2 | 71738 | MAM domain containing 2 | 259 | 150 | 1.73 | 0.016 |
| Enpp2 | 18606 | ectonucleotide pyrophosphatase/phosphodiesterase 2 | 400 | 231 | 1.73 | 0.000 |
| 5033413D22Rik | 75973 | RIKEN cDNA 5033413D22 gene | 81 | 47 | 1.73 | 0.005 |
| 3110047P20Rik | 319807 | RIKEN cDNA 3110047P20 gene | 151 | 88 | 1.72 | 0.004 |
| Ttc21a | 74052 | Tetratricopeptide repeat domain 21A | 45 | 26 | 1.72 | 0.016 |
| C1s /// | 100044326 | complement component 1, s subcomponent /// similar | 145 | 85 | 1.72 | 0.005 |
| LOC100044326 | /// 50908 | to Complement component 1, s subcomponent | | | | |
| Ccdc39 | 61938 | coiled-coil domain containing 39 | 24 | 14 | 1.70 | 0.012 |
| Kcnq1ot1 | 63830 | KCNQ1 overlapping transcript 1 | 482 | 284 | 1.70 | 0.018 |
| 1110069O07Rik | 71798 | RIKEN cDNA 1110069O07 gene | 286 | 169 | 1.70 | 0.003 |
| — | — | — | 90 | 54 | 1.68 | 0.025 |
| Armc3 | 70882 | armadillo repeat contaning 3 | 96 | 58 | 1.68 | 0.037 |
| Morn3 | 74890 | MORN repeat containing 3 | 47 | 28 | 1.67 | 0.015 |
| Ttc25 | 74407 | tetratricopeptide repeat domain 25 | 82 | 49 | 1.67 | 0.013 |
| Ccdc39 | 51938 | coiled-coil domain containing 39 | 308 | 185 | 1.67 | 0.009 |
| Enpp2 | 18606 | ectonucleotide pyrophosphatase/phosphodiesterase 2 | 1041 | 625 | 1.67 | 0.000 |
| Dnahc6 | 330355 | dynein, axonemal, heavy chain 6 | 137 | 83 | 1.66 | 0.045 |
| 6430537H07Rik | 226265 | RIKEN cDNA 6430537H07 gene | 89 | 54 | 1.65 | 0.003 |
| Tsnaxip1 | 72236 | translin-associated factor X (Tsnax) interacting protein 1 | 95 | 57 | 1.65 | 0.005 |
| Nudt6 | 229228 | nudix (nucleoside diphosphate linked moiety X) type | 667 | 404 | 1.65 | 0.002 |
| | | motif 6 | | | | |
| Mylk | 107589 | myosin, light polypeptide kinase | 845 | 516 | 1.64 | 0.014 |
| 1700001C02Rik | 75434 | RIKEN cDNA 1700001C02 gene | 76 | 46 | 1.64 | 0.018 |
| Serping1 | 12258 | serine (or cysteine) peptidase inhibitor, clade G, member 1 | 444 | 272 | 1.63 | 0.000 |
| Rasa4 | 54153 | RAS p21 protein activator 4 | 99 | 61 | 1.63 | 0.001 |
| Tgfb2 | 21808 | transforming growth factor, beta 2 | 1160 | 714 | 1.62 | 0.000 |
| Rsph10b2 | 75136 | radial spoke head 10 homolog B (Chlamydomonas) | 40 | 25 | 1.62 | 0.045 |
| Zbtb16 | 235320 | zinc finger and BTB domain containing 16 | 73 | 45 | 1.62 | 0.002 |
| Sorcs1 | 58178 | VPS10 domain receptor protein SORCS 1 | 29 | 18 | 1.61 | 0.003 |
| A430071A18Rik | 319454 | RIKEN cDNA A430071A18 gene | 139 | 86 | 1.61 | 0.004 |
| — | — | — | 75 | 47 | 1.60 | 0.021 |
| — | — | — | 39 | 25 | 1.60 | 0.028 |
| Iqca | 74918 | IQ motif containing with AAA domain | 54 | 34 | 1.60 | 0.010 |
| Grid2 | 14804 | glutamate receptor, ionotropic, delta 2 | 74 | 47 | 1.59 | 0.007 |
| Adamts1 | 11504 | a disintegrin-like and metallopeptidase (reprolysin type) | 921 | 582 | 1.58 | 0.019 |
| | | with thrombospondin type 1 motif, 1 | | | | |
| Tmem107 | 66910 | transmembrane protein 107 | 2806 | 1774 | 1.58 | 0.005 |
| Adamts5 /// | 100048332 /// | a disintegrin-like and metallopeptidase (reprolysin type) | 154 | 97 | 1.58 | 0.000 |
| LOC100048332 | 23794 | with thrombospondin type 1 motif, 5 (aggrecanase-2) /// | | | | |
| | | similar to a disintegrin-like and metalloprotease | | | | |
| | | (reprolysin type) with thrombospondin type 1 motif, 5 | | | | |
| | | (aggrecanase-2) | | | | |
| Col5a3 | 53867 | collagen, type V, alpha 3 | 113 | 72 | 1.58 | 0.000 |
| Gm11992 | 626870 | predicted gene 11992 | 22 | 14 | 1.58 | 0.009 |
| S100a6 | 20200 | S100 calcium binding protein A6 (calcyclin) | 1545 | 982 | 1.57 | 0.001 |
| — | — | — | 70 | 45 | 1.57 | 0.029 |
| Atp1b2 | 11932 | ATPase, Na+/K+ transporting, beta 2 polypeptide | 283 | 180 | 1.57 | 0.000 |
| Dkk2 | 56811 | dickkopf homolog 2 (Xenopus laevis) | 862 | 550 | 1.57 | 0.000 |
| 1700094D03Rik | 73545 | RIKEN cDNA 1700094D03 gene | 178 | 114 | 1.56 | 0.001 |
| Efemp1 | 216616 | epidermal growth factor-containing fibulin-like | 916 | 586 | 1.56 | 0.001 |
| | | extracellular matrix protein 1 | | | | |
| — | — | — | 89 | 57 | 1.56 | 0.023 |
| — | — | — | 18 | 12 | 1.55 | 0.028 |
| Tgfbr3 | 21814 | transforming growth factor, beta receptor III | 1182 | 762 | 1.55 | 0.027 |
| Hspa4l | 18415 | heat shock protein 4 like | 217 | 140 | 1.55 | 0.034 |
| Fbxl20 | 72194 | F-box and leucine-rich repeat protein 20 | 140 | 91 | 1.54 | 0.017 |
| Lrrc46 | 69297 | leucine rich repeat containing 46 | 175 | 114 | 1.54 | 0.026 |
| Arhgap6 | 11856 | Rho GTPase activating protein 5 | 74 | 48 | 1.54 | 0.001 |
| Lrguk | 74354 | leucine-rich repeats and guanylate kinase domain | 22 | 14 | 1.53 | 0.007 |
| | | containing | | | | |
| 2310007A19Rik | 66353 | RIKEN cDNA 2310007A19Rik | 119 | 78 | 1.52 | 0.032 |
| Neat1 | 66961 | nuclear paraspeckle assembly transcript 1 (non-protein | 386 | 254 | 1.52 | 0.038 |
| | | coding) | | | | |
| Ttc21a | 74052 | Tetratricopeptide repeat domain 21A | 71 | 47 | 1.52 | 0.010 |
| Ppp1r3c | 63412 | protein phosphatase 1, regulatory (inhibitor) subunit 3C | 816 | 537 | 1.52 | 0.013 |
| Spag16 | 66722 | sperm associated antigen 16 | 67 | 44 | 1.52 | 0.034 |
| 9130014G24Rik | 215772 | RIKEN cDNA 9130014G24 gene | 12 | 8 | 1.51 | 0.001 |
| Rcan2 | 53901 | regulator of calcineurin 2 | 237 | 157 | 1.51 | 0.000 |
| Adm | 11535 | adrenomedullin | 149 | 99 | 1.51 | 0.002 |
| Pax3 | 18505 | paired box gene 3 | 271 | 180 | 1.51 | 0.003 |
| Pcdh9 | 211712 | protocadherin 9 | 161 | 107 | 1.51 | 0.012 |
| 84galt7 | 218271 | xylosylprotein beta1,4-galactosyltransferase, | 74 | 49 | 1.51 | 0.025 |
| | | polypeptide 7 (galactosyltransferase 1) | | | | |
| AbI3bp | 320712 | ABI gene family, member 3 (NESH) binding protein | 282 | 187 | 1.50 | 0.007 |
| 1700029J07Rik | 69479 | RIKEN cDNA 1700029J07 gene | 53 | 35 | 1.50 | 0.040 |
| Zfp474 | 66758 | zinc finger protein 474 | 70 | 47 | 1.50 | 0.004 |

TABLE 2
Downregulated genes in the palate of Tgfbr2^{fl/fl}; Wnt1-Cre mice at E14.5

| | | | CKO Geo | WT Geo | CKO/WT | |
|---|---|---|---|---|---|---|
| Symbol | GeneID | Gene Title | Mean | Mean | ratio | FDR |
| Tacc3 | 21335 | transforming, acidic coiled-coil containing protein 3 | 288 | 435 | −1.51 | 0.000 |
| Limo1 | 29806 | LIM domains containing 1 | 473 | 717 | −1.51 | 0.000 |
| Ndc80 | 67052 | NDC80 homolog, kinetochore complex component (S. cerevisiae) | 191 | 289 | −1.51 | 0.002 |
| Kif23 | 71819 | kinesin family member 23 | 120 | 182 | −1.52 | 0.005 |
| Rrm2 | 20135 | ribonucleotide reductase M2 | 574 | 872 | −1.52 | 0.001 |
| Fgf9 | 14180 | fibroblast growth factor 9 | 74 | 112 | −1.52 | 0.011 |
| Spc25 | 66442 | SPC25, NDC80 kinetochore complex component, homolog | 637 | 967 | −1.52 | 0.005 |
| | | (S. cerevisiae) | | | | |
| Slc40a1 | 53945 | solute carrier family 40 (iron-regulated transporter), member 1 | 195 | 296 | −1.52 | 0.006 |
| Cenph | 26886 | centromere protein H | 247 | 376 | −1.52 | 0.002 |
| Bub1 | 12235 | budding uninhibited by benzimidazoles 1 homolog (S. cerevisiae) | 85 | 129 | −1.52 | 0.000 |
| 2610017I09Rik | 66297 | RIKEN cDNA 2610017I09 gene | 268 | 409 | −1.53 | 0.000 |
| Spc24 | 67629 | SPC24, NDC80 kinetochore complex component, homolog | 463 | 707 | −1.53 | 0.000 |
| | | (S. cerevisiae) | | | | |
| Hs3st1 | 15476 | heparan sulfate (glucosamine) 3-O-sulfotransferase 1 | 107 | 163 | −1.53 | 0.002 |
| Smc6 | 67241 | structural maintenance of chromosomes 6 | 56 | 101 | −1.53 | 0.037 |
| Blrc5 | 11799 | baculoviral IAP repeat-containing 5 | 895 | 1367 | −1.53 | 0.000 |
| Car2 | 12349 | carbonic anhydrase 2 | 217 | 331 | −1.53 | 0.019 |
| Adcy8 | 11514 | adenylate cyclase 8 | 79 | 121 | −1.53 | 0.029 |
| Shcbp1 | 20419 | Shc SH2-domain binding protein 1 | 735 | 1125 | −1.53 | 0.031 |
| Depdc1b | 218581 | DEP domain containing 1B | 149 | 228 | −1.53 | 0.000 |
| Mmrn1 | 70945 | multimerin 1 | 119 | 182 | −1.53 | 0.000 |
| Fen1 | 14156 | flap structure specific endonuclease 1 | 310 | 475 | −1.53 | 0.000 |
| Traip | 22036 | TRAF-interacting protein | 106 | 163 | −1.53 | 0.020 |
| Cdc25c | 12532 | cell division cycle 25 homolog C (S. pombe) | 113 | 173 | −1.54 | 0.001 |
| Irx2 /// | 100045612 | iroquols related homeobox 2 (Drosophila) /// similar to iroquois-class | 39 | 59 | −1.54 | 0.018 |
| LOC100045612 | /// 15372 | homeobox protein IRX2 | | | | |
| Spsb4 | 211949 | splA/ryanodine receptor domain and SOCS box containing 4 | 34 | 52 | −1.54 | 0.002 |
| Cdc45l | 12544 | cell division cycle 45 homolog (S. cerevisiae)-like | 145 | 224 | −1.54 | 0.000 |
| Fli1 | 14247 | Friend leukemia integration 1 | 128 | 198 | −1.54 | 0.014 |
| Ccna2 | 12428 | cyclin A2 | 915 | 1415 | −1.55 | 0.000 |
| Esco2 | 71988 | establishment of cohesion 1 homolog 2 (S. cerevisiae) | 206 | 318 | −1.55 | 0.026 |
| 4632434I11Rik | 74041 | RIKEN cDNA 4632434I11 gene | 145 | 224 | −1.55 | 0.024 |
| Gas2 | 14453 | growth arrest specific 2 | 352 | 547 | −1.55 | 0.001 |
| Gins1 | 69270 | GINS complex subunit 1 (Psf1 homolog) | 337 | 525 | −1.56 | 0.002 |
| Kif20b | 240641 | kinesin family member 20B | 302 | 470 | −1.56 | 0.004 |
| Exo1 | 26909 | exonuclease 1 | 98 | 153 | −1.56 | 0.001 |
| — | — | — | 53 | 82 | −1.56 | 0.005 |
| 1200009O22Rik | 66873 | RIKEN cDNA 1200009O22 gene | 196 | 306 | −1.56 | 0.005 |
| Kif11 | 16551 | kinesin family member 11 | 271 | 424 | −1.56 | 0.002 |
| Hmgb2 | 97165 | high mobility group box 2 | 59 | 92 | −1.56 | 0.012 |
| Hmga2 | 15364 | high mobility group AT-hook 2 | 1174 | 1840 | −1.57 | 0.017 |
| Fbxo5 | 67141 | F-box protein 5 | 490 | 758 | −1.57 | 0.007 |
| Pitx2 | 18741 | paired-like homeodomain transcription factor 2 | 719 | 1129 | −1.57 | 0.043 |
| Bub1b | 12236 | budding uninhibited by benzimidazoles 1 homolog, beta (S. cerevisiae) | 393 | 617 | −1.57 | 0.000 |
| Eme1 | 268465 | essential melotic endonuclease 1 homolog 1 (S. pombe) | 159 | 251 | −1.57 | 0.000 |
| Tk1 | 21877 | thymidine kinase 1 | 407 | 640 | −1.57 | 0.000 |
| Rad51ap1 | 19362 | RAD51 associated protein 1 | 88 | 139 | −1.58 | 0.003 |
| Cd55 | 13136 | CD55 antigen | 72 | 114 | −1.58 | 0.001 |
| Ncapg | 54392 | non-SMC condensin I complex, subunit G | 216 | 342 | −1.58 | 0.002 |
| Bub1 | 12235 | budding uninhibited by benzimidazoles 1 homolog (S. cerevisiae) | 652 | 1030 | −1.58 | 0.002 |
| Sgol1 | 72415 | shugoshin-like 1 (S. pombe) | 175 | 278 | −1.58 | 0.000 |
| Kif22 | 110033 | kinesin family member 22 | 357 | 565 | −1.58 | 0.000 |
| Pf4 | 56744 | platelet factor 4 | 264 | 419 | −1.58 | 0.016 |
| Ccnb1 /// Gm5593 | 268697 /// | cyclin B1 /// predicted gene 5593 /// predicted gene 8416 | 560 | 887 | −1.58 | 0.000 |
| Gm8416 | 434175 /// | | | | | |
| | 667005 | | | | | |
| C79407 | 217653 | expressed sequence C79407 | 301 | 477 | −1.58 | 0.007 |
| Myl3 | 17897 | myosin, light polypeptide 3 | 22 | 35 | −1.59 | 0.006 |
| Ccdc99 | 70385 | coiled-coil domain containing 99 | 259 | 411 | −1.59 | 0.000 |
| Slc40a1 | 53945 | solute carrier family 40 (iron-regulated transporter), member 1 | 220 | 350 | −1.59 | 0.000 |
| Cdca5 | 67849 | cell division cycle associated 5 | 208 | 330 | −1.59 | 0.000 |
| Cdca3 | 14793 | cell division cycle associated 3 | 592 | 1102 | −1.59 | 0.000 |
| 2610036L11Rik | 66312 | RIKEN cDNA 2610036L11 gene | 129 | 206 | −1.60 | 0.007 |
| Sertad4 | 214791 | SERTA domain containing 4 | 836 | 1335 | −1.60 | 0.000 |
| Gmnn | 57441 | geminin | 527 | 843 | −1.60 | 0.001 |
| Tyms /// Tyms-ps | 22171 /// | thymidylate synthase /// thymidylate synthase, pseudogene | 568 | 912 | −1.61 | 0.008 |
| | 22172 | | | | | |
| 2810433K01Rik | 66468 | RIKEN cDNA 2810433K01 gene | 170 | 274 | −1.61 | 0.008 |
| Mtm1 | 17772 | X-linked myotubular myopathy gene 1 | 629 | 1012 | −1.61 | 0.025 |
| Cdc6 | 23834 | cell division cycle 6 homolog (S. cerevisiae) | 205 | 331 | −1.61 | 0.000 |
| Kifc1 /// | 100044746 | kinesin family member C1 /// similar to Kifc1 protein | 139 | 224 | −1.61 | 0.002 |
| LOC100044746 | /// 16580 | | | | | |
| Epha4 | 13838 | Eph receptor A4 | 30 | 48 | −1.62 | 0.041 |
| Spsb4 | 211949 | splA/ryanodine receptor domain and SOCS box containing 4 | 73 | 118 | −1.62 | 0.000 |
| Fam70a | 245386 | family with sequence similarity 70, member A | 158 | 257 | −1.62 | 0.039 |
| Cdca5 | 67849 | cell division cycle associated 5 | 462 | 751 | −1.62 | 0.000 |
| Hs3st6 | 328779 | heparan sulfate (glucosamine) 3-O-sulfotransferase 6 | 100 | 163 | −1.63 | 0.008 |
| Glmap4 | 107526 | GTPase, IMAP family member 4 | 169 | 275 | −1.63 | 0.000 |
| Ppil5 | 69706 | peptidylprolyl isomerase (cyclophilin) like 5 | 202 | 329 | −1.63 | 0.005 |
| Nptx1 | 18164 | neuronal pentrakin 1 | 22 | 35 | −1.63 | 0.045 |
| Cyp26b1 | 232174 | cytochrome P450, family 26, subfamily b, polypeptide 1 | 239 | 390 | −1.63 | 0.000 |
| Grem2 | 23893 | gremlin 2 homolog, cysteine knot superfamily (Xenopus laevis) | 199 | 327 | −1.64 | 0.000 |
| Hmga2 | 15364 | high mobility group AT-hook 2 | 1473 | 2418 | −1.64 | 0.014 |
| Kif22 | 110033 | kinesin family member 22 | 159 | 262 | −1.64 | 0.000 |
| Angptl1 | 72713 | angiopoietin-like 1 | 283 | 466 | −1.65 | 0.000 |
| Cdc25c | 12532 | cell division cycle 25 homolog C (S. pombe) | 92 | 152 | −1.65 | 0.006 |
| Mpped2 | 77015 | metallophosphoesterase domain containing 2 | 458 | 759 | −1.66 | 0.011 |
| Exo1 | 26909 | exonuclease 1 | 180 | 300 | −1.66 | 0.000 |
| Nasp | 50927 | nuclear autoantigenic sperm protein (histone-binding) | 156 | 260 | −1.67 | 0.001 |
| Tnfsl11 | 21943 | tumor necrosis factor (ligand) superfamily, member 11 | 19 | 31 | −1.69 | 0.023 |
| 2010317E24Rik | 72080 | RIKEN cDNA 2010317E24 gene | 52 | 88 | −1.69 | 0.014 |
| Etv5 | 104156 | ets variant gene 5 | 173 | 292 | −1.69 | 0.011 |
| Kbtbd5 | 72330 | kelch repeat and BTB (POZ) domain containing 5 | 47 | 79 | −1.69 | 0.035 |
| Hist1h3a /// | 15077 /// | histone cluster 1, H3a /// histone cluster 1, H3b /// histone | 228 | 387 | −1.70 | 0.000 |
| Hist1h3b /// | 260423 /// | cluster 1, H3c /// histone cluster 1, H3d /// histone cluster 1, | | | | |
| Hist1h3c /// | 319148 /// | H3e /// histone cluster 1, H3f /// histone cluster 1, H3g /// | | | | |
| Hist1h3d /// | 319149 /// | histone cluster 1, H3h /// histone cluster 1, H3i /// | | | | |
| Hist1h3e /// | 319150 /// | histone cluster 2, H3b /// histone cluster 2, H3c1 /// histone | | | | |
| Hist1h3f /// | 319151 /// | cluster 2, H3c2 | | | | |
| Hist1h3g /// | 319152 /// | | | | | |
| Hist1h3h /// | 319153 /// | | | | | |
| Hist1h3i /// | 319154 /// | | | | | |
| Hist2h3b /// | 360198 /// | | | | | |
| Hist2h3c1 /// | 97114 /// | | | | | |
| Hist2h3c2 | 97908 | | | | | |
| D2Ertd750e | 51944 | DNA segment, Chr 2, ERATO Dol 750, expressed | 188 | 319 | −1.70 | 0.000 |
| Dscc1 | 72107 | defective in sister chromatid cohesion 1 homolog (S. cerevisiae) | 210 | 358 | −1.70 | 0.025 |
| Angptl1 | 72713 | angiopoietin-like 1 | 169 | 290 | −1.71 | 0.000 |
| Cdkn3 | 72891 | cyclin-dependent kinase inhibitor 3 | 124 | 215 | −1.73 | 0.015 |
| H2afv | 77605 | H2A histone family, member V | 1662 | 2880 | −1.73 | 0.013 |

TABLE 3
Mass spectrometry analysis in the MEPM cells of
Tgfbr2^{fl/fl}; /Wnt1-Cre mice

| | | | Peptides | |
|---|---|---|---|---|
| | Mass | Score | matched | Protein |
| #1 | 32883 | 285 | 16 | Solute carrier family 25 |
| | 32230 | 227 | 23 | Similar to 14-3-3z protein |
| | 27754 | 186 | 22 | 14-3-3z protein |
| | 28814 | 141 | 8 | Phosphoglycerate mutase 1 |
| | 28069 | 126 | 17 | 14-3-3b protein |
| | 28285 | 104 | 20 | 14-3-3g protein |
| | 20848 | 92 | 9 | H1 histone family, member O |
| #2 | 274052 | 1153 | 104 | spectrin beta 2 isoform 1 |
| | 269665 | 669 | 46 | talin 1 |
| | 280325 | 639 | 62 | filamin alpha |
| | 272368 | 621 | 51 | fibronectin 1 |
| | 285170 | 451 | 34 | spectrin alpha 2 |
| | 41766 | 365 | 38 | actin gamma |
| | 272257 | 265 | 16 | fatty acid synthase |

TABLE 4
Upregulated genes in the palate of Tgfbr2fl/fl; Wnt1-Cre mice at E14.5

| | | | CKO | WT | | |
|---|---|---|---|---|---|---|
| | | | Geo | Geo | CKO/WT | |
| Symbol | GeneID | Gene Title | Mean | Mean | Ratio | FDR |
| Snad1 | 208777 | sushi, nidogen and EGF-like domains 1 | 1668 | 417 | 4.00 | 0.001 |
| — | — | — | 315 | 81 | 3.88 | 0.013 |
| Meig1 | 104362 | meiosis expressed gene 1 | 270 | 77 | 3.51 | 0.009 |
| — | — | — | 204 | 62 | 3.30 | 0.020 |
| Krt13 | 16663 | keratin 13 | 389 | 129 | 3.02 | 0.023 |
| 4833427G06Rik | 235345 | RIKEN cDNA 4833427G06 gene | 310 | 109 | 2.84 | 0.007 |
| Hdc | 15186 | histidine decarboxylase | 172 | 62 | 2.78 | 0.009 |
| Lrrc23 | 16977 | leucine rich repeat containing 23 | 651 | 243 | 2.68 | 0.003 |
| Dynlrb2 | 75465 | dynein light chain roadblock-type 2 | 1287 | 482 | 2.67 | 0.012 |
| — | — | — | 35 | 13 | 2.63 | 0.023 |
| Ccdc153 | 270150 | coiled-coil domain containing 153 | 103 | 40 | 2.58 | 0.006 |
| Fam154b | 330577 | family with sequence similarity 154, member B | 88 | 33 | 2.57 | 0.001 |
| Dnaho12 /// | 110083 /// | dynein, axonemal, heavy chain 12 /// dynein, | 77 | 30 | 2.55 | 0.019 |
| Dnahc7i | 207778 | axonemal, heavy chain 7-like | | | | |
| C030048H21Rik | 77481 | RIKEN cDNA C030048H21 gene | 111 | 44 | 2.52 | 0.005 |
| — | — | — | 121 | 50 | 2.43 | 0.002 |
| Morn5 | 75495 | MORN repeat containing 5 | 191 | 79 | 2.40 | 0.010 |
| Ak7 | 78801 | adenylate kinase 7 | 203 | 86 | 2.36 | 0.002 |
| 9830021D06Rik | 319926 | RIKEN cDNA 9630021D06 gene | 284 | 122 | 2.32 | 0.024 |
| — | — | — | 261 | 113 | 2.30 | 0.013 |
| Hdc | 15186 | histidine decarboxylase | 235 | 103 | 2.29 | 0.010 |
| Ccdc108 | 241116 | coiled-coil domain containing 108 | 104 | 46 | 2.28 | 0.000 |
| E030011K20Rik | 208613 | RIKEN cDNA E030011K20 gene | 99 | 44 | 2.28 | 0.034 |
| Rsph1 | 22092 | radial spoke head 1 homolog (Chiamydomonas) | 393 | 173 | 2.27 | 0.022 |
| — | — | — | 407 | 179 | 2.27 | 0.044 |
| 1110069O07Rik | 71798 | RIKEN cDNA 1110069O07 gene | 327 | 144 | 2.27 | 0.004 |
| Wdr63 | 242253 | WD repeat domain 63 | 89 | 40 | 2.26 | 0.017 |
| 4921509J17Rik | 70857 | RIKEN cDNA 4921509J17 gene | 291 | 132 | 2.20 | 0.011 |
| Ccdc67 | 234964 | coiled-coil domain containing 67 | 952 | 439 | 2.17 | 0.031 |
| 1700012B09Rik | 69325 | RIKEN cDNA 1700012B09 gene | 105 | 49 | 2.16 | 0.009 |
| Agt | 11606 | angiotensinogen (serpin peptidase inhibitor, | 179 | 83 | 2.16 | 0.000 |
| | | clade A, member 8) | | | | |
| Ube2d2 | 56550 | ubiquitin-conjugating enzyme E2D 2 | 196 | 91 | 2.16 | 0.019 |
| — | — | — | 114 | 53 | 2.16 | 0.042 |
| 1110069O07Rik | 71798 | RIKEN cDNA 1110069O07 gene | 316 | 147 | 2.15 | 0.001 |
| Ttc29 | 73301 | tetratricopeptide repeat domain 29 | 56 | 26 | 2.14 | 0.033 |
| Tekt4 | 71840 | tektin 4 | 132 | 62 | 2.11 | 0.032 |
| Dnali1 | 75563 | dynein, axonemal, light intermediate polypeptide 1 | 148 | 70 | 2.10 | 0.003 |
| Vit | 74199 | vitrin | 166 | 70 | 2.09 | 0.000 |
| Tgfb2 | 21808 | transforming growth factor, beta 2 | 295 | 143 | 2.07 | 0.001 |
| Krt13 | 16663 | keratin 13 | 25 | 12 | 2.02 | 0.001 |
| Ndufa4l2 | 407790 | NADH dehydrogenase (ubiquinone) 1 alpha | 1080 | 534 | 2.02 | 0.001 |
| | | subcomplex, 4-like 2 | | | | |
| 1700009P17Rik | 75472 | RIKEN cDNA 1700009P17 gene | 723 | 360 | 2.01 | 0.040 |
| Pde4d | 238871 | phosphodiesterase 4D, cAMP specific | 43 | 22 | 2.00 | 0.048 |
| Zmynd10 | 114602 | zinc finger, MYND domain containing 10 | 239 | 121 | 1.98 | 0.032 |
| Dnahc2 | 327954 | dynein, axonemal, heavy chain 2 | 128 | 65 | 1.97 | 0.008 |
| Traf1 | 22029 | TNF recepter-associated factor 1 | 78 | 40 | 1.98 | 0.000 |
| Ube3a | 22215 | ubiquitin protein ligase E3A | 205 | 105 | 1.96 | 0.033 |
| Tgfb2 | 21808 | transforming growth factor, beta 2 | 1262 | 645 | 1.95 | 0.002 |
| 6820408C15Rik | 228778 | RIKEN cDNA 6820408C15 gene | 178 | 92 | 1.94 | 0.009 |
| 1700094D03Rik | 73545 | RIKEN cDNA 1700094D03 gene | 570 | 296 | 1.92 | 0.002 |
| Pde4d | 238871 | phosphodiesterase 4D, cAMP specific | 43 | 22 | 2.00 | 0.048 |
| Zmynd10 | 114602 | zinc finger, MYND domain containing 10 | 239 | 121 | 1.98 | 0.032 |
| Dnahc2 | 327954 | dynein, axonemal, heavy chain 2 | 128 | 65 | 1.97 | 0.008 |
| Traf1 | 22029 | TNF receptor-associated factor 1 | 78 | 40 | 1.96 | 0.000 |
| Ube3a | 22215 | ubiquitin protein ligase E3A | 205 | 105 | 1.96 | 0.033 |
| Tgfb2 | 21808 | transforming growth factor, beta 2 | 1282 | 645 | 1.95 | 0.002 |
| 6820408C15Rik | 228778 | RIKEN cDNA 6820408C15 gene | 178 | 92 | 1.94 | 0.009 |
| 1700094D03Rik | 73545 | RIKEN cDNA 1700094D03 gene | 570 | 296 | 1.92 | 0.002 |
| Slitrk1 | 76955 | SLIT and NTRK-like family, member 1 | 104 | 54 | 1.92 | 0.017 |
| 4933404M02Rik | 66748 | RIKEN cDNA 4933404M02 gene | 54 | 28 | 1.92 | 0.004 |
| Grid2 | 14804 | glutamate receptor, Ionotropic, delta 2 | 128 | 68 | 1.89 | 0.001 |
| Krt4 | 16682 | keratin 4 | 356 | 188 | 1.89 | 0.039 |
| Krt4 | 16682 | keratin 4 | 282 | 149 | 1.89 | 0.006 |
| Abca8a | 217258 | ATP-binding cassette, sub-family A (ABC1), | 337 | 179 | 1.88 | 0.000 |
| | | member 8a | | | | |
| Akap14 | 434756 | A kinase (PRKA) anchor protein 14 | 81 | 43 | 1.88 | 0.012 |
| — | — | — | 150 | 80 | 1.88 | 0.044 |
| D19Ertd652e | 70806 | DNA segment, Chr 19, ERATO Doi 652, expressed | 74 | 40 | 1.87 | 0.000 |
| D19Ertd652e | 70806 | DNA segment, Chr 19, ERATO Doi 652, expressed | 25 | 13 | 1.87 | 0.011 |
| Tekt1 | 21689 | tektin 1 | 373 | 199 | 1.87 | 0.039 |
| Psma3 | 19167 | Proteasome (prosome, macropain) subunit, alpha | 207 | 112 | 1.84 | 0.013 |
| | | type 3 | | | | |
| Tppp3 | 67971 | tubulin polymerization-promoting protein family | 608 | 330 | 1.84 | 0.036 |
| | | member 3 | | | | |
| — | — | — | 87 | 47 | 1.84 | 0.010 |
| Nme5 | 75533 | non-metastatic cells 5, protein expressed in | 401 | 219 | 1.83 | 0.032 |
| | | (nucleoside-diphosphate kinase) | | | | |
| Serpine1 | 18787 | serine (or cysteine) peptidase inhibitor, clade E, | 90 | 49 | 1.83 | 0.000 |
| | | member 1 | | | | |
| Dnahc7b | 227058 | dynein, axonemal, heavy chain 7B | 87 | 48 | 1.82 | 0.011 |
| 6430537H07Rik | 226265 | RIKEN cDNA 6430537H07 gene | 135 | 75 | 1.81 | 0.040 |
| Mdh1b | 76668 | malate dehydrogenase 1B, NAD (soluble) | 45 | 25 | 1.81 | 0.037 |
| C230072F16Rik | 320784 | RIKEN cDNA G230072F16 gene | 76 | 42 | 1.80 | 0.004 |
| Hspa4l | 18415 | heat shock protein 4 like | 326 | 182 | 1.79 | 0.017 |
| 1600029D21Rik | 76509 | RIKEN cDNA 1600029D21 gene | 167 | 93 | 1.79 | 0.020 |
| Krt13 | 16663 | keratin 13 | 62 | 35 | 1.79 | 0.050 |
| Cdh7 | 241201 | cadherin 7, type 2 | 116 | 64 | 1.78 | 0.014 |
| 4932425I24Rik | 320214 | RIKEN cDNA 4932425I24 gene | 63 | 35 | 1.78 | 0.010 |
| Adamts5 /// | 100048332 | a disintegrin-like and metallopeptidase (reprolysin | 328 | 185 | 1.78 | 0.000 |
| LOC100048332 | /// 23794 | type) with thrombospondin type 1 motif, 5 | | | | |
| | | (aggrecanase-2) /// similar to a disintegrin-like and | | | | |
| | | metalloprotease (reprolysin type) with | | | | |
| | | thrombospondin type 1 motif, 5 (aggrecanase-2) | | | | |
| 2900006K08Rik | 72873 | RIKEN cDNA 2900006K08 gene | 155 | 87 | 1.78 | 0.041 |
| A330021E22Rik | 207686 | RIKEN cDNA A330021E22 gene | 174 | 98 | 1.77 | 0.001 |
| D14Abb1e | 218850 | DNA segment, Chr 14, Abbolt 1 expressed | 383 | 216 | 1.77 | 0.028 |
| Tgfb2 | 21808 | transforming growth factor, beta 2 | 957 | 544 | 1.76 | 0.000 |
| Dio3os | 353504 | deiodinase, iodothyronine type III, opposite strand | 384 | 218 | 1.76 | 0.000 |
| Hspa4l | 18415 | heat shock protein 4 like | 112 | 64 | 1.75 | 0.012 |
| Kndc1 | 76484 | kinase non-catalytic C-lobe domain (KIND) | 108 | 62 | 1.75 | 0.007 |
| | | containing 1 | | | | |
| 1600029D21Rik | 76509 | RIKEN cDNA 1600029D21 gene | 130 | 75 | 1.74 | 0.016 |
| — | — | — | 66 | 38 | 1.74 | 0.003 |
| Mamdc2 | 71738 | MAM domain containing 2 | 259 | 150 | 1.73 | 0.016 |
| Enpp2 | 18606 | ectonucleotide | 400 | 231 | 1.73 | 0.000 |
| | | pyrophosphatase/phosphodiesterase 2 | | | | |
| 5033413D22Rik | 75973 | RIKEN cDNA 5033413D22 gene | 81 | 47 | 1.73 | 0.005 |
| 3110047P20Rik | 319807 | RIKEN cDNA 3110047P20 gene | 151 | 88 | 1.72 | 0.004 |
| Ttc21a | 74052 | Tetratricopeptide repeat domain 21A | 45 | 26 | 1.72 | 0.016 |
| C1s /// | 100044326 | complement component 1, s subcomponent /// | 145 | 85 | 1.72 | 0.005 |
| LOC100044326 | /// 50908 | similar to Complement component 1, s | | | | |
| | | subcomponent | | | | |
| Ccdc39 | 51938 | coiled-coil domain containing 39 | 24 | 14 | 1.70 | 0.012 |
| Kcnq1ot1 | 63830 | KCNQ1 overlapping transcript 1 | 482 | 284 | 1.70 | 0.018 |
| 1110069O07Rik | 71798 | RIKEN cDNA 1110069O07 gene | 286 | 169 | 1.70 | 0.003 |
| — | — | — | 90 | 54 | 1.68 | 0.025 |
| Armc3 | 70882 | armadillo repeat containing 3 | 96 | 58 | 1.68 | 0.037 |
| Morn3 | 74890 | MORN repeat containing 3 | 47 | 28 | 1.67 | 0.015 |
| Ttc25 | 74407 | tetratricopeptide repeat domain 25 | 82 | 49 | 1.67 | 0.013 |
| Ccdc39 | 51938 | coiled-coil domain containing 39 | 308 | 185 | 1.67 | 0.009 |
| Enpp2 | 18606 | ectonucleotide | 1041 | 625 | 1.67 | 0.000 |
| | | pyrophosphatase/phosphodiesterase 2 | | | | |
| Dnahc6 | 330355 | dynein, axonemal, heavy chain 6 | 137 | 83 | 1.66 | 0.045 |
| 6430537H07Rik | 226265 | RIKEN cDNA 6430537H07 gene | 89 | 54 | 1.65 | 0.003 |
| Tsnaxip1 | 72236 | translin-associated factor X (Tsnax) interacting | 95 | 57 | 1.65 | 0.005 |
| | | protein 1 | | | | |
| Nudt6 | 229228 | nudix (nucleoside diphosphate linked moiety X)- | 667 | 404 | 1.65 | 0.002 |
| | | type motif 6 | | | | |
| Mylk | 107589 | myosin, light polypeptide kinase | 845 | 516 | 1.64 | 0.014 |
| 1700001C02Rik | 75434 | RIKEN cDNA 1700001C02 gene | 76 | 46 | 1.64 | 0.018 |
| Serping1 | 12258 | serine (or cysteine) peptidase inhibitor, clade G, | 444 | 272 | 1.63 | 0.000 |
| | | member 1 | | | | |
| Rasa4 | 54153 | RAS p21 protein activator 4 | 99 | 61 | 1.63 | 0.001 |
| Tgfb2 | 21808 | transforming growth factor, beta 2 | 1160 | 714 | 1.62 | 0.000 |
| Rsph10b2 | 75136 | radial spoke head 10 homolog B (Chlamydomonas) | 40 | 25 | 1.62 | 0.045 |
| Zbtb16 | 235320 | zinc finger and BTB domain containing 16 | 73 | 45 | 1.62 | 0.002 |
| Sorcs1 | 58178 | VPS10 domain receptor protein SORCS 1 | 29 | 18 | 1.61 | 0.003 |
| A430071A18Rik | 319454 | RIKEN cDNA A430071A18 gene | 139 | 86 | 1.61 | 0.004 |
| — | — | — | 75 | 47 | 1.60 | 0.021 |
| — | — | — | 39 | 25 | 1.60 | 0.028 |
| Iqca | 74918 | IQ motif containing with AAA domain | 54 | 34 | 1.60 | 0.010 |
| Grid2 | 14804 | glutamate receptor, ionotropic, delta 2 | 74 | 47 | 1.59 | 0.007 |
| Adamts1 | 11504 | a disintegrin-like and metallopeptidase (reprolysin | 921 | 582 | 1.58 | 0.019 |
| | | type) with thrombospondin type 1 motif, 1 | | | | |
| Tmem107 | 86910 | transmembrane protein 107 | 2806 | 1774 | 1.58 | 0.005 |
| Adamts5 /// | 100048332 | a disintegrin-like and metallopeptidase (reprolysin | 154 | 97 | 1.58 | 0.000 |
| LOC100048332 | /// 23794 | type) with thrombospondin type 1 motif, 5 | | | | |
| | | (aggrecanase-2) /// similar to a disintegrin-like and | | | | |
| | | metalloprotease (reprolysin type) with | | | | |
| | | thrombospondin type 1 motif, 5 (aggrecanase-2) | | | | |
| Col5a3 | 53867 | collagen, type V, alpha 3 | 113 | 72 | 1.68 | 0.000 |
| Gm11992 | 626870 | predicted gene 11992 | 22 | 14 | 1.58 | 0.009 |
| S100a6 | 20200 | S100 calcium binding protein A6 (calcyclin) | 1545 | 982 | 1.57 | 0.001 |
| — | — | — | 70 | 45 | 1.57 | 0.029 |
| Atp1b2 | 11932 | ATPase, Na+/K+ transporting, beta 2 polypeptide | 283 | 180 | 1.57 | 0.000 |
| Dkk2 | 56811 | dickkopf homolog 2 (Xenopus laevis) | 862 | 550 | 1.57 | 0.000 |
| 1700094D03Rik | 73545 | RIKEN cDNA 1700094D03 gene | 178 | 114 | 1.56 | 0.001 |
| Efemp1 | 216616 | epidermal growth factor-containing fibulin-like | 916 | 586 | 1.56 | 0.001 |
| | | extracellular matrix protein 1 | | | | |
| — | — | — | 89 | 57 | 1.56 | 0.023 |
| — | — | — | 18 | 12 | 1.55 | 0.028 |
| Tgfbr3 | 21814 | transforming growth factor, beta receptor III | 1182 | 762 | 1.55 | 0.027 |
| Hspa4l | 18415 | heal shock protein 4 like | 217 | 140 | 1.55 | 0.034 |
| Fbxl20 | 72194 | F-box and leucine-rich repeat protein 20 | 140 | 91 | 1.54 | 0.017 |
| Lrrc46 | 69297 | leucine rich repeat containing 46 | 175 | 114 | 1.54 | 0.026 |
| Arhgap6 | 11856 | Rho GTPase activating protein 6 | 74 | 48 | 1.54 | 0.001 |
| Lrguk | 74354 | leucine-rich repeats and guanylate kinase domain | 22 | 14 | 1.53 | 0.007 |
| | | containing | | | | |
| 2310007A19Rik | 66353 | RIKEN cDNA 2310007A19Rik | 119 | 78 | 1.52 | 0.032 |
| Neat1 | 66961 | nuclear paraspeckle assembly transcript 1 (non- | 386 | 254 | 1.52 | 0.038 |
| | | protein coding) | | | | |
| Ttc21a | 74052 | Tetratricopeptide repeat domain 21A | 71 | 47 | 1.52 | 0.010 |
| Ppp1r3c | 53412 | protein phosphatase 1, regulatory (inhibitor) subunit | 816 | 537 | 1.52 | 0.013 |
| | | 3C | | | | |
| Spag16 | 66722 | sperm associated antigen 16 | 67 | 44 | 1.52 | 0.034 |
| 9130014G24Rik | 215772 | RIKEN cDNA 9130014G24 gene | 12 | 8 | 1.51 | 0.001 |
| Rcan2 | 53901 | regulator of calcineurin 2 | 237 | 157 | 1.51 | 0.000 |
| Adm | 11535 | adrenomedullin | 149 | 99 | 1.51 | 0.002 |
| Pax3 | 18505 | paired box gene 3 | 271 | 180 | 1.51 | 0.003 |
| Pcdh9 | 211712 | protocadherin 9 | 161 | 107 | 1.51 | 0.012 |
| B4galt7 | 218271 | xylosylprotein beta 1,4-galactosyltransferase, | 74 | 49 | 1.51 | 0.025 |
| | | polypeptide 7 (galactosyltransferase 1) | | | | |
| Abi3bp | 320712 | ABI gene family, member 3 (NESH) binding protein | 282 | 187 | 1.50 | 0.007 |
| 1700029J07Rik | 69479 | RIKEN cDNA 1700029J07 gene | 53 | 35 | 1.50 | 0.040 |
| Zfp474 | 66758 | zinc finger protein 474 | 70 | 47 | 1.50 | 0.004 |

TABLE 5
Downregulated genes in the palate of Tgfbr^{fl/fl}; Wnt1-Cre mice at E14.5

| | | | CKO | WT | CKO/ | |
|---|---|---|---|---|---|---|
| | | | Geo | Geo | WT | |
| Symbol | GeneID | Gene Title | Mean | Mean | ratio | FDR |
| Tacc3 | 21335 | transforming, acidic coiled-coil containing protein 3 | 288 | 435 | −1.51 | 0.000 |
| Limd1 | 29806 | LIM domains containing 1 | 473 | 717 | −1.51 | 0.000 |
| Ndc80 | 67052 | NDC80 homolog, kinetochore complex component (S. cerevisiae) | 191 | 289 | −1.51 | 0.002 |
| Kif23 | 71819 | kinesin family member 23 | 120 | 182 | −1.52 | 0.005 |
| Rrm2 | 20135 | ribonucleotide reductase M2 | 574 | 872 | −1.52 | 0.001 |
| Fgf9 | 14180 | fibroblast growth factor 9 | 74 | 112 | −1.52 | 0.011 |
| Spc25 | 66442 | SPC25, NDC80 kinetochore complex component, homolog (S. cerevisiae) | 637 | 967 | −1.52 | 0.006 |
| Slc40a1 | 53945 | solute carrier family 40 (Iron-regulated transporter), member 1 | 195 | 296 | −1.52 | 0.006 |
| Cenph | 26886 | centromere protein H | 247 | 376 | −1.52 | 0.002 |
| Bub1 | 12235 | budding uninhibited by benzimidazoles 1 homolog (S. cerevisiae) | 85 | 129 | −1.52 | 0.000 |
| 2610017I09Rik | 66297 | RIKEN cDNA 2610017I09 gene | 268 | 409 | −1.53 | 0.000 |
| Spc24 | 67629 | SPC24, NDC80 kinetochore complex component, homolog (S. cerevisiae) | 463 | 707 | −1.53 | 0.000 |
| Hs3st1 | 15476 | heparan sulfate (glucosamine) 3-O-sulfotransferase 1 | 107 | 163 | −1.53 | 0.002 |
| Smc6 | 67241 | structural maintenance of chromosomes 6 | 66 | 101 | −1.53 | 0.037 |
| Birc5 | 11799 | baculoviral IAP repeat-containing 5 | 895 | 1367 | −1.53 | 0.000 |
| Car2 | 12349 | carbonic anhydrase 2 | 217 | 331 | −1.53 | 0.019 |
| Adcy8 | 11514 | adenylate cyclase 8 | 79 | 121 | −1.53 | 0.029 |
| Shcbp1 | 20419 | Shc SH2-domain binding protein 1 | 735 | 1125 | −1.53 | 0.031 |
| Depdc1b | 218581 | DEP domain containing 1B | 149 | 228 | −1.53 | 0.000 |
| Mmrn1 | 70945 | multimerin 1 | 119 | 182 | −1.53 | 0.000 |
| Fen1 | 14156 | flap structure specific endonuclease 1 | 310 | 475 | −1.53 | 0.000 |
| Tralp | 22036 | TRAF-interacting protein | 106 | 163 | −1.53 | 0.020 |
| Cdc25c | 12532 | cell division cycle 25 homolog C (S. pombe) | 113 | 173 | −1.54 | 0.001 |
| Irx2 /// | 100045612 | Iroquois related homeobox 2 (Drosophila) /// similar to iroquois-class | 39 | 59 | −1.54 | 0.018 |
| LOC100045612 | /// 16372 | homeobox protein IRX2 | | | | |
| Spsb4 | 211949 | splA/tyanodine receptor domain and SOCS box containing 4 | 34 | 52 | −1.54 | 0.002 |
| Cdc45l | 12544 | cell division cycle 45 homolog (S. cerevisiae)-like | 145 | 224 | −1.54 | 0.000 |
| Fli1 | 14247 | Friend leukemia integration 1 | 128 | 198 | −1.54 | 0.014 |
| Ccna2 | 12428 | cyclin A2 | 915 | 1415 | −1.55 | 0.000 |
| Esco2 | 71988 | establishment of cohesion 1 homolog 2 (S. cerevisiae) | 206 | 318 | −1.55 | 0.026 |
| 4632434I11Rik | 74041 | RIKEN cDNA 4632434I11 gene | 145 | 224 | −1.55 | 0.024 |
| Gas2 | 14453 | growth arrest specific 2 | 352 | 647 | −1.55 | 0.001 |
| Gins1 | 69270 | GINS complex subunit 1 (Psf1 homolog) | 337 | 525 | −1.56 | 0.002 |
| Kif20b | 240641 | kinesin family member 20B | 302 | 470 | −1.56 | 0.004 |
| Exo1 | 26909 | exonuclease 1 | 98 | 153 | −1.56 | 0.001 |
| — | — | — | 53 | 82 | −1.56 | 0.005 |
| 1200009O22Rik | 66873 | RIKEN cDNA 1200009O22 gene | 196 | 306 | −1.56 | 0.006 |
| Kif11 | 16551 | kinesin family member 11 | 271 | 424 | −1.56 | 0.002 |
| Hmgb2 | 97165 | high mobility group box 2 | 59 | 92 | −1.56 | 0.012 |
| Hmga2 | 15364 | high mobility group AT-hook 2 | 1174 | 1840 | −1.57 | 0.017 |
| Fbxo5 | 67141 | F-box protein 5 | 490 | 768 | −1.57 | 0.007 |
| Pitx2 | 18741 | paired-like homeodomain transcription factor 2 | 719 | 1129 | −1.57 | 0.043 |
| Bub1b | 12236 | budding uninhibited by benzimidazoles 1 homolog, beta (S. cerevisiae) | 393 | 617 | −1.57 | 0.000 |
| Eme1 | 268465 | essential melotic endonuclease 1 homolog 1 (S. pombe) | 159 | 251 | −1.57 | 0.000 |
| Tk1 | 21877 | thymidine kinase 1 | 407 | 640 | −1.57 | 0.000 |
| Rad51ap1 | 19362 | RAD51 associated protein 1 | 88 | 139 | −1.58 | 0.003 |
| Cd55 | 13136 | CD55 antigen | 72 | 114 | −1.58 | 0.001 |
| Ncapg | 54392 | non-SMC condensin I complex, subunit G | 216 | 342 | −1.58 | 0.002 |
| Bub1 | 12235 | budding uninhibited by benzimidazoles 1 homolog (S. cerevisiae) | 652 | 1030 | −1.58 | 0.002 |
| Sgol1 | 72415 | shugoshin-like 1 (S. pombe) | 175 | 278 | −1.58 | 0.000 |
| Kif22 | 110033 | kinesin family member 22 | 357 | 565 | −1.58 | 0.000 |
| Pf4 | 56744 | platelet factor 4 | 264 | 419 | −1.58 | 0.016 |
| Ccnb1 /// Gm5593 | 268697 /// | cyclin B1 /// predicted gene 5593 /// predicted gene 8416 | 560 | 887 | −1.58 | 0.000 |
| /// Gm8416 | 434175 /// | | | | | |
| | 667005 | | | | | |
| C79407 | 217653 | expressed sequence C79407 | 301 | 477 | −1.58 | 0.007 |
| Myl3 | 17897 | myosin, light polypeptide 3 | 22 | 35 | −1.59 | 0.006 |
| Ccdc99 | 70385 | coiled-coil domain containing 99 | 259 | 411 | −1.59 | 0.000 |
| Slc40a1 | 53945 | solute carrier family 40 (Iron-regulated transporter), member 1 | 220 | 350 | −1.59 | 0.000 |
| Cdca5 | 67849 | cell division cycle associated 5 | 208 | 330 | −1.59 | 0.000 |
| Cdca3 | 14793 | cell division cycle associated 3 | 692 | 1102 | −1.59 | 0.000 |
| 2610036L11Rik | 66311 | RIKEN cDNA 2610038L11 gene | 129 | 206 | −1.60 | 0.007 |
| Sertad4 | 214791 | SERTA domain containing 4 | 836 | 1335 | −1.60 | 0.000 |
| Gmnn | 57441 | geminin | 527 | 843 | −1.60 | 0.001 |
| Tyms /// Tyms-ps | 22171 /// | thymidylate synthase /// thymidylate synthase, pseudogene | 568 | 912 | −1.61 | 0.008 |
| | 22172 | | | | | |
| 2810433K01Rik | 66468 | RIKEN cDNA 2810433K01 gene | 170 | 274 | −1.61 | 0.008 |
| Mtm1 | 17772 | X-linked myotubular myopathy gene 1 | 629 | 1012 | −1.61 | 0.025 |
| Cdc6 | 23834 | cell division cycle 6 homolog (S. cerevisiae) | 205 | 331 | −1.61 | 0.000 |
| Kifc1 /// | 100044746 | kinesin family member C1 /// similar to Kifc1 protein | 139 | 224 | −1.61 | 0.002 |
| LOC100044746 | /// 16580 | | | | | |
| Epha4 | 13838 | Eph receptor A4 | 30 | 48 | −1.62 | 0.041 |
| Spsb4 | 211949 | splA/ryanodine receptor domain and SOCS box containing 4 | 73 | 118 | −1.62 | 0.000 |
| Fam70a | 245386 | family with sequence similarity 70, member A | 158 | 257 | −1.62 | 0.039 |
| Cdca5 | 67849 | cell division cycle associated 5 | 462 | 751 | −1.62 | 0.000 |
| Hs3st6 | 328779 | heparan sulfate (glucosamine) 3-O-sulfotransferase 6 | 100 | 163 | −1.63 | 0.008 |
| Gimap4 | 107526 | GTPase, IMAP family member 4 | 169 | 275 | −1.63 | 0.000 |
| Ppil5 | 69706 | peptidylprolyl isomerase (cyclophilin) like 5 | 202 | 329 | −1.63 | 0.005 |
| Nptx1 | 18164 | neuronal pentraxin 1 | 22 | 35 | −1.63 | 0.045 |
| Cyp26b1 | 232174 | cytochrome P450, family 26, subfamily b, polypeptide 1 | 239 | 390 | −1.63 | 0.000 |
| Grem2 | 23893 | gremlin 2 homolog, cysteine knot superfamily (Xenopus laevis) | 199 | 327 | −1.64 | 0.000 |
| Hmga2 | 15364 | high mobility group AT-hook 2 | 1473 | 2418 | −1.64 | 0.014 |
| Kif22 | 110033 | kinesin family member 22 | 159 | 262 | −1.64 | 0.000 |
| Angptl1 | 72713 | angiopoietin-like 1 | 283 | 466 | −1.65 | 0.000 |
| Cdc25c | 12532 | cell division cycle 25 homolog C (S. pombe) | 92 | 152 | −1.65 | 0.006 |
| Mpped2 | 77015 | metallophosphoesterase domain containing 2 | 458 | 759 | −1.65 | 0.011 |
| Exo1 | 26909 | exonuclease 1 | 180 | 300 | −1.66 | 0.000 |
| Nasp | 50927 | nuclear autoantigenic sperm protein (histone-binding) | 156 | 260 | −1.67 | 0.001 |
| Tnfsf11 | 21943 | tumor necrosis factor (ligand) superfamily, member 11 | 19 | 31 | −1.69 | 0.023 |
| 2010317E24Rik | 72080 | RIKEN cDNA 2010317E24 gene | 52 | 88 | −1.69 | 0.014 |
| Etv5 | 104156 | ets variant gene 5 | 173 | 292 | −1.69 | 0.011 |
| Kbtbd5 | 72330 | kelch repeat and BTB (POZ) domain containing 5 | 47 | 79 | −1.69 | 0.035 |
| Hist1h3a /// | 15077 /// | histone cluster 1, H3a /// histone cluster 1, H3b /// histone cluster 1, | 228 | 387 | −1.70 | 0.000 |
| Hist1h3b /// | 260423 | H3c /// histone cluster 1, H3d /// histone cluster 1, H3e /// histone | | | | |
| Hist1h3c /// | /// | cluster 1, H3f /// histone cluster 1, H3g /// histone cluster 1, H3h /// | | | | |
| Hist1h3d /// | 319148 | histone cluster 1, H3i /// histone cluster 2, H3b /// histone cluster 2, | | | | |
| Hist1h3e /// | /// | H3c1 /// histone cluster 2, H3c2 | | | | |
| Hist1h3f /// | 319149 | | | | | |
| Hist1h3g /// | /// | | | | | |
| Hist1h3h /// | 319150 | | | | | |
| Hist1h3i /// | /// | | | | | |
| Hist2h3b /// | 319151 | | | | | |
| Hist2h3c1 /// | /// | | | | | |
| Hist2h3c2 | 319152 | | | | | |
| | /// | | | | | |
| | 319153 /// | | | | | |
| | 319154 /// | | | | | |
| | 360198 | | | | | |
| | /// 97114 | | | | | |
| | /// 97908 | | | | | |
| D2Er1d750e | 51944 | DNA segment, Chr 2, ERATO Doi 750, expressed | 188 | 319 | −1.70 | 0.000 |
| Dscc1 | 72107 | defective in sister chromatid cohesion 1 homolog (S. cerevisiae) | 210 | 358 | −1.70 | 0.025 |
| Angptl1 | 72713 | angiopoietin-like 1 | 169 | 290 | −1.71 | 0.000 |
| Cdkn3 | 72391 | cyclin-dependent kinase inhibitor 3 | 124 | 215 | −1.73 | 0.015 |
| H2afv | 77605 | H2A histone family, member V | 1662 | 2880 | −1.73 | 0.013 |
| Pde1a | 18573 | phosphodiesterase 1A, calmodulin-dependent | 79 | 138 | −1.74 | 0.015 |
| Pitx2 | 18741 | paired-like homeodomain transcription factor 2 | 224 | 389 | −1.74 | 0.006 |
| — | — | — | 29 | 51 | −1.74 | 0.013 |
| Lars2 | 102436 | leucyl-tRNA synthetase, mitochondrial | 139 | 242 | −1.74 | 0.000 |
| Fam70a | 245386 | family with sequence similarity 70, member A | 237 | 414 | −1.74 | 0.000 |
| Rasf11b | 68939 | RAS-like, family 11, member B | 839 | 1505 | −1.79 | 0.000 |
| Klhl4 | 237010 | kelch-like 4 (Drosophila) | 178 | 320 | −1.80 | 0.013 |
| 1200009O22Rik | 65873 | RIKEN cDNA 1200009O22 gene | 302 | 550 | −1.82 | 0.000 |
| Dusp9 | 75590 | dual specificity phosphatase 9 | 29 | 54 | −1.83 | 0.000 |
| H2afv | 77605 | H2A histone family, member V | 84 | 165 | −1.84 | 0.032 |
| Gsg2 | 14841 | germ cell-specific gene 2 | 75 | 140 | −1.86 | 0.016 |
| Fbln5 | 23876 | fibulin 5 | 795 | 1491 | −1.88 | 0.000 |
| LOC100048362 | 100048362 | similar to mKIAA1238 protein /// ribosomal modification protein rimK- | 63 | 120 | −1.91 | 0.004 |
| /// Rimklb | /// 108653 | like family member B | | | | |
| Hfe2 | 69585 | hemochromalosis type 2 (juvenile) (human homolog) | 52 | 101 | −1.93 | 0.028 |
| — | — | — | 29 | 56 | −1.94 | 0.010 |
| Shisa2 | 219134 | shisa homolog 2 (Xenopus laevis) | 110 | 223 | −2.02 | 0.003 |
| Hmga2 | 15364 | high mobility group AT-hook 2 | 423 | 863 | −2.04 | 0.046 |
| Calca | 12310 | calcitonin/calcitonin-related polypeptide, alpha | 59 | 125 | −2.11 | 0.023 |
| 2010315B03Rik | 630836 | RIKEN cDNA 2010315B03 gene | 42 | 89 | −2.12 | 0.003 |
| Dusp9 | 75590 | dual specificity phosphatase 9 | 19 | 40 | −2.12 | 0.000 |
| — | — | — | 18 | 39 | −2.14 | 0.000 |
| D730035F11Rik | 320010 | RIKEN cDNA D730035F11 gene | 59 | 126 | −2.14 | 0.001 |
| Crym | 12971 | crystallin, mu | 61 | 132 | −2.15 | 0.027 |
| Ablim3 | 319713 | actin binding LIM protein family, member 3 | 33 | 71 | −2.16 | 0.018 |
| Srl | 106393 | sarcalumenin | 143 | 315 | −2.20 | 0.029 |
| Thbs4 | 21828 | thrombospondin 4 | 63 | 139 | −2.22 | 0.008 |
| Ctla2b | 13025 | cytotoxic T lymphocyte-associated protein 2 beta | 33 | 75 | −2.25 | 0.005 |
| Cdh4 | 12561 | cadherin 4 | 118 | 269 | −2.28 | 0.000 |
| Cdh4 | 12561 | cadherin 4 | 94 | 225 | −2.40 | 0.000 |
| Vwa2 | 240675 | von Willebrand factor A domain containing 2 | 62 | 126 | −2.42 | 0.001 |
| Myh6 /// Myh7 | 140781 | myosin, heavy polypeptide 6, cardiac muscle, alpha /// myosin, heavy | 25 | 60 | −2.43 | 0.001 |
| | /// 17888 | polypeptide 7, cardiac muscle, beta | | | | |
| Anp32a | 11737 | acidic (leucine-rich) nuclear phosphoprotein 32 family, member A | 502 | 1223 | −2.44 | 0.005 |
| Fabp4 | 11770 | fatty acid binding protein 4, adipocyte | 19 | 45 | −2.45 | 0.005 |
| Tnmd | 64103 | tenomodulin | 90 | 225 | −2.51 | 0.025 |
| Lmod3 | 320502 | leiomodin 3 (fetal) | 14 | 36 | −2.55 | 0.028 |
| Myoz2 | 59006 | myozenin 2 | 43 | 116 | −2.69 | 0.018 |
| Fabp4 | 11770 | fatty acid binding protein 4, adipocyte | 30 | 83 | −2.79 | 0.000 |
| Tln | 22138 | titln | 58 | 174 | −2.98 | 0.029 |
| Tnnt1 | 21955 | troponin T1, skeletal, slow | 147 | 467 | −3.18 | 0.030 |
| Myom2 | 17930 | myomesin 2 | 26 | 83 | −3.19 | 0.034 |
| Myf5 | 17877 | myogenic factor 5 | 14 | 44 | −3.20 | 0.004 |
| Smyd1 | 12180 | SET and MYND domain containing 1 | 11 | 37 | −3.24 | 0.014 |
| Myh7 | 140781 | myosin, heavy polypeptide 7, cardiac muscle, beta | 47 | 152 | −3.25 | 0.000 |
| Myom2 | 17930 | myomesin 2 | 24 | 92 | −3.77 | 0.011 |
| Actn2 | 11472 | actinin alpha 2 | 75 | 283 | −3.77 | 0.026 |
| Myot | 58916 | myoillin | 19 | 71 | −3.85 | 0.015 |
| Myom2 | 17930 | myomesin 2 | 41 | 188 | −4.56 | 0.014 |
| Sln | 66402 | sarcolipin | 41 | 220 | −5.35 | 0.024 |
| Smpx | 66106 | small muscle protein, X-linked | 19 | 105 | −5.41 | 0.009 |
| Tceal7 | 1E+08 | transcription elongation factor A (SII)-like 7 | 24 | 186 | −7.74 | 0.014 |
| Lztfl1 | 93730 | leucine zipper transcription factor-like 1 | 11 | 105 | −9.53 | 0.000 |

TABLE 6
Mass spectrometry analsysi in the MEPM
cells of Tgfbr2^{fl/fl}; Wnt1-Cre mice

| | | | Peptides | |
|---|---|---|---|---|
| | Mass | Score | matched | Protein |
| #1 | 32883 | 285 | 16 | Solute carrier family 25 |
| | 32230 | 227 | 23 | Similar to 14-3-3ζ protein |
| | 27754 | 186 | 22 | 14-3-3ζ protein |
| | 28814 | 141 | 8 | Phosphoglycerate mutase 1 |
| | 28069 | 126 | 17 | 14-3-3β protein |
| | 28285 | 104 | 20 | 14-3-3γ protein |
| | 20848 | 92 | 9 | H1 histone family, member O |
| #2 | 274052 | 1153 | 104 | spectrin beta 2 isoform 1 |
| | 269665 | 669 | 46 | talin 1 |
| | 280325 | 639 | 62 | filamin alpha |
| | 272368 | 621 | 51 | fibronectin 1 |
| | 285170 | 451 | 34 | spectrin alpha 2 |
| | 41766 | 365 | 38 | actin gamma |
| | 272257 | 265 | 16 | fatty acid synthase |

What is claimed is:

1. A method for diagnosing developmental craniofacial malformation in a developing subject, comprising:
determining an expression level of Tgf-β2 and/or ectopic p38 MAPK activation;
comparing said Tgf-β2 level and/or said p38 MAPK activation to suitable control levels,
wherein when said level of Tgf-β2 and/or ectopic p38 MAPK activation in said subject is higher than the control level, said subject is diagnosed as being at risk of craniofacial malformation.

2. The method of claim 1, wherein said determining step determines both the level of Tgf-β2 and ectopic p38 MAPK activation.

3. The method of claim 2, wherein said subject is only diagnosed as being at risk of craniofacial malformation when both Tgf-β2 and ectopic p38 MAPK activation are higher than their respective control levels.

4. The method of claim 1, wherein said subject is a human fetus.

5. The method of claim 1, wherein said Tgf-β2 level and ectopic p38 MAPK activation are obtained from cranial neural crest cells of the subject.

6. A method for treating or preventing craniofacial malformation in a subject in need of said treatment, comprising:
administering to said subject an effective amount of a Tgf-β2 or p38 MAPK inhibitor or modulator,
wherein said subject is a fetus and said administering can be either direct administration to the fetus or indirect administration via the fetus' mother while the fetus is being carried by the mother.

7. The method of claim 6, wherein said p38 MAPK inhibitor or modulator is 4-[4-(4-fluorophenyl)-2-(4-memylsufinylphenyl)-7H-imidazol-5-yl]pyridine.

8. The method of claim 6, wherein said Tgf-β2 inhibitor or modulator is selected from the group consisting of losartan, telmisartan, and combinations thereof.

9. A method for treating craniofacial malformation in a subject in need of said treatment, comprising:
administering to said subject an effective amount of an antibody or an antibody fragment for Tgf-βRIII, Tgf-β2, or a combination thereof,
wherein said subject is a fetus and said administering can be either direct administration to the fetus or indirect administration via the fetus' mother while the fetus is being carried by the mother.

10. A genetically engineered animal model for craniofacial malformation, comprising:
a genetically engineered mouse carrying double mutations in Msx1 and Dlx5.

11. A method for identifying compounds that may be potential drugs for treating craniofacial malformation, comprising:
administering a test compound to a suitable animal model with elevated expression level of Tgf-β2 and/or ectopic p38 MAPK activation; and
determining the level of Tgf-β2 and/or p38 MAPK activity,
wherein if the level of Tgf-β2 and/or p38 MAPK activation are reduced more than a predetermined amount, said test compound is identified as a potential lead compound.

12. The method of claim 11, further comprising the step of observing development of the animal model, wherein if the animal does not develop a craniofacial malformation, the test compound is further identified as a lead compound.

13. A composition for treating craniofacial malformation, comprising:
Losartan, telmisartan, 4-[4-(4-fluorophenyl)-2-(4-methylsufinylphenyl)-1H-imidazol-5-yl]pyridine, an antibody or antibody fragment for Tgf-βRIII or Tgf-β2, or any combination thereof; and
a pharmaceutically acceptable carrier.


SEQUENCE LISTING (4 sequences)

SEQ ID NO: 1 - DNA, 20 bp, Mus musculus
cggctaccacatccaaggaa

SEQ ID NO: 2 - DNA, 18 bp, Mus musculus
gctggaattaccgcggct

SEQ ID NO: 3 - DNA, 22 bp, Mus musculus
ctctacaggtcatgcttccacc

SEQ ID NO: 4 - DNA, 21 bp, Mus musculus
acagaacagtcttctcaccct